(19) United States
(12) Patent Application Publication
(10) Pub. No.: US 2017/0073763 A1
(43) Pub. Date: Mar. 16, 2017

(54) Methods and Compositions for Assessing Patients with Non-small Cell Lung Cancer

(71) Applicant: The Board of Trustees of the Leland Stanford Junior University, Stanford, CA (US)

(72) Inventors: Maximilian Diehn, San Carlos, CA (US); Andrew J. Gentles, Menlo Park, CA (US); Arash Ash Alizadeh, San Mateo, CA (US); Scott Victor Bratman, Toronto (CA)

(21) Appl. No.: 15/123,497

(22) PCT Filed: Mar. 12, 2015
(86) PCT No.: PCT/US15/20244

§ 371 Date: Sep. 2, 2016

Related U.S. Application Data
(60) Provisional application No. 61/952,057, filed on Mar. 12, 2014.

(51) Int. Cl. C12Q 1/68

(52) U.S. Cl. CPC C12Q 1/6886; C12Q 2600/118; C12Q 2600/158

(57) ABSTRACT

Methods, kits, devices, and computer systems are provided for obtaining an NSCLC marker level representation for an individual with non small cell lung carcinoma (NSCLC); and/or for providing a prognosis for an individual with NSCLC. The methods can include measuring expression levels, in a biological sample, of 2 or more NSCLC markers selected from: MAD2L1, GINS1, SLC2A1, KRT6A, FCGRT, TNIK, BCAM, KDM6A, and FAIM3; calculating an NSCLC marker level representation based on the measured expression levels; comparing the NSCLC marker level representation of the individual to a reference marker level representation; providing a prognosis based on the comparison; and/or generating a report that includes at least one of: (i) an NSCLC marker level representation, (ii) an NSCLC marker level representation and a reference NSCLC marker level representation, (iii) a prognosis, and (iv) guidance to a clinician as to a treatment recommendation based on the prognosis.

### CROSS REFERENCE

This application is a 371 application and claims the benefit of PCT Application No. PCT/US2015/020244, filed Mar. 12, 2015, which claims benefit of U.S. Provisional Patent Application No. 61/952,057, filed Mar. 12, 2014, which applications are incorporated herein by reference in their entirety.

### GOVERNMENT RIGHTS

This invention was made with government support under contracts CA147933 and CA154969 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a text file, “STAN-1023_Seq List_ST25.txt” created on Feb. 14, 2014 and having a size of 251 KB. The contents of the text file are incorporated by reference herein in their entirety.

### BACKGROUND OF THE INVENTION

Lung cancer is the leading global cause of cancer death, and non-small cell lung cancer (NSCLC) accounts for ˜85% of all lung cancer cases. Even when diagnosed in early stages before involving lymph nodes or distant sites, surgical resection cures only ˜60% of NSCLC cases. Efforts to improve the survival rate for stage I NSCLC by the administration of adjuvant chemotherapy have been largely unsuccessful. Therefore, robust methods for risk stratification in early stage NSCLC are needed in order to inform clinical decisions. To maximize the potential benefit of adjuvant chemotherapy in early stage NSCLC, treatment would ideally only be administered to groups at high risk of disease recurrence and death from lung cancer. While a variety of clinical, pathological, and molecular/biological features have been proposed for risk stratification in early stage NSCLC, to date no method has been incorporated into routine clinical practice.

Differences in gene expression between tumors can reflect important attributes of tumor biology and provide prognostic information in a variety of cancer types. Successful applications of gene expression analysis have yielded clinical tools with potential value for oncologists, for example for breast cancer, colon cancer, prostate cancer, and non-Hodgkin lymphoma. A number of studies have proposed similar tools for predicting prognosis in early stage NSCLC. However, efforts to translate gene expression-based prognostic methods into clinical use for NSCLC have been met by a number of pitfalls, including overfitting, lack of sufficient internal and external validation, histological heterogeneity of NSCLC (e.g., squamous cell carcinoma, adenocarcinoma, etc.), predominance of immune infiltrates in many tumors, intratumoral heterogeneity, and lack of accounting for existing clinical variables that stratify outcome, among others. The use of gene expression analyses to identify high-risk groups specifically within stage I NSCLC has proven especially challenging.

There is need in the art for robust methods for risk stratification of NSCLC (e.g., in early stage NSCLC) patients in order to inform clinical decisions.

### Publications

Yang, et al., J Biol Chem. 2013 Feb. 1; 288(5):2965-75; Kato et al., J Surg Oncol. 2012 Sep. 15; 106(4):423-30; Karpathiou, et al., APMIS. 2013 July; 121(7):592-604; Sasaki et al., Mol Med Rep. 2012 March; 5(3):599-602; Younes et al., Cancer. 1997 Sep. 15; 80(6):1046-51; Zhao, et al., Cancer Lett. 2010 Apr. 28; 290(2):238-47; Dejmek et al., Oncol Rep. 2006 March; 15(3):583-7; Camilo et al., Hum Pathol. 2006 May; 37(5):542-6; Patent application numbers US20120295803, US20090076098, US20040063120, US20120329662, US20060172305, US20060172305, US20040023267, US20100184034, US20120190567, US20120225954, US20110053156, WO2012090073, US20120207708, US20110097717, US20090149333, US20090215054, US20100216795; and U.S. Pat. Nos. 8,202,968; and 7,585,634.

### SUMMARY OF THE INVENTION

Methods, kits, devices, and computer systems are provided for obtaining an NSCLC marker level representation for an individual with non small cell lung carcinoma (NSCLC). Aspects of the methods include measuring expression levels in a biological sample from the individual (e.g., a tumor sample), of 2 or more NSCLC markers. In some embodiments the markers are selected where two or more genes are selected from Autosome clusters 1-4 (as shown in Table 8). In some embodiments two or more genes are selected from any of the autosome clusters. In some embodiments at least one gene is selected at least one autosome cluster. In some embodiments the markers are selected from: MAD2L1, GINS1, SLC2A1, KRT6A, FCGRT, TNIK, BCAM, KDM6A, and FAIM3. An NSCLC marker level representation is calculated based on the measured expression levels. In some cases, calculating an NSCLC marker level representation comprises normalizing (e.g., determining the measured expression levels of the 2 or more NSCLC markers relative to the expression levels of one or more housekeeping genes) and/or weighting the measured expression levels of the 2 or more NSCLC markers.

In some cases, the NSCLC marker level representation is an NSCLC score. In some cases, calculating an NSCLC score includes normalizing and/or weighting the expression levels of the 2 or more NSCLC markers and calculating a molecular prognostic index (MPI) value that represents a combination of the normalized and/or weighted expression levels. In some cases, calculating the MPI value includes adding, subtracting, and weighting the expression levels according to the formula: 4y₁+4y₂+5y₃+4y₄−5y₅−5y₆−5y₇−5y₈−6y₉, where each of y₁ to y₉ are the expression levels (e.g., normalized expression levels) of the corresponding NSCLC marker, where y₁ is MAD2L1, y₂ is GINS1, y₃ is SLC2A1, y₄ is KRT6A, y₅ is FCGRT, y₆ is TNIK, y₇ is BCAM, y₈ is KDM6A, and y₉ is FAIM3.

In some cases, calculating an NSCLC score includes integrating clinical data from the individual with the MPI value of the individual to obtain a composite risk model (CRM) value. In some cases, the methods include generating a report that includes the NSCLC marker level representation of the individual. In some cases, the report includes a reference NSCLC marker level representation. In some cases, the methods include inputting the expression levels of the 2 or more NSCLC markers into a computer comprising a processor programmed to perform the calculating step. In some cases, the NSCLC is a non-squamous NSCLC. In some cases, the NSCLC is a stage I NSCLC.

Methods, kits, devices, and computer systems are also provided for providing a prognosis for an individual with NSCLC. Aspects of the methods include obtaining an NSCLC marker level representation for the individual (as described above), comparing the NSCLC marker level representation of the individual to a reference marker level representation (e.g., derived from an experimentally determined data set comprising survival data and NSCLC marker level representation data for individuals with NSCLC), and providing a prognosis for the individual based on the comparison. In general, the expression levels of MAD2L1, GINS1, SLC2A1, and KRT6A correlate negatively with a positive prognosis (e.g., increased expression correlates with increased risk/lower survival likelihood and/or decreased expression correlates with decreased risk/higher survival likelihood); and the expression levels of FCGRT, TNIK, BCAM, KDM6A, and FAIM3 correlate positively with a positive prognosis (e.g., increased expression correlates with decreased risk/higher survival likelihood and/or decreased expression correlates with increased risk/lower survival likelihood). In some cases, the reference marker level representation is a threshold marker level representation. In some cases, the reference marker level representation is a predetermined threshold (e.g., a predetermined threshold score). In some cases, the prognosis is a category of survival likelihood (e.g., high risk, intermediate risk, low risk, and the like, e.g., where high risk is associated with a low likelihood of survival, low risk is associated with a high likelihood of survival, and medium risk is associated with an intermediate likelihood of survival). In some cases, the prognosis is a statistical likelihood of survival.

In some cases, the methods include generating a report that includes at least one of: (i) the NSCLC marker level representation of the individual, (ii) the NSCLC marker level representation of the individual and a reference NSCLC marker level representation, (iii) the prognosis, and (iv) guidance to a clinician as to a treatment recommendation for the individual based on the prognosis. In some cases, the methods include inputting the expression levels of the 2 or more NSCLC markers into a computer comprising a processor programmed to perform at least one of: the calculating step, the comparing step, and the providing step. In some cases, the computer generates the report.

In some cases, the methods include providing a treatment regimen based on the prognosis (e.g., providing an aggressive treatment regimen, e.g., chemotherapy, if the predicted likelihood of survival is below a predetermined threshold). In some cases, the methods include administering and/or prescribing a treatment (e.g., the recommended treatment).

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1: depict Identification and selection of prognostic genes from gene expression datasets of stage 1 non-squamous NSCLC. (A) Schematic representation of the study design for prognostic gene discovery and validation. (B) AutoSOME clustering of the top 1012 prognostic genes in the training cohort reveals at least 4 dominant clusters comprising 71% (n=716) of the genes; significant cluster annotations are indicated in Table 5. (C) Kaplan-Meier survival analysis in the training cohort; cases were split into “high” and “low” cohorts according the median of the integrated expression of the genes in each of the 4 largest clusters from (B).

FIG. 2: MPI genes are expressed in different cellular populations within NSCLC tumors. (A-F) Whole transcriptome sequencing (RNA-Seq) of sorted lung adenocarcinoma cell populations representing epithelial cells (EpCAM+CD45−CD31−), immune cells (CD45+), endothelial cells (CD31+CD45−), and stromal cells (CD10+CD45−CD31−EpCAM−). FPKM for each of the MPI genes is shown for the distinct cell populations. (A) Average FPKM of genes in each cluster; (B) Cluster 1 genes; (C) Cluster 2 genes; (D) Cluster 3 genes; (E) Cluster 4 gene. N=4 tumors. Error bars: S.E.M. (F) Tumor epithelial cells and infiltrating immune cells provide most of the total expression for the MPI genes. The fraction of the total gene expression within a tumor that is contributed by each of the sorted cell types is shown. (G, H) Forest plot depicting univariate hazard ratios and 95% confidence intervals for death in the microarray training set according to expression levels of the indicated genes (G) or according to the indicated clinical variables (H).

FIG. 3: A 9-gene expression-based MPI predicts survival in stage 1 non-squamous NSCLC. (A,B) Kaplan-Meier survival analysis of the test set from the microarray meta-cohort with stratification of risk groups based on the median value of the MPI. (C,D) Kaplan-Meier survival analysis of the validation qPCR cohort with risk groups defined by the median MPI value in the training set.

FIG. 4: A Composite Risk Model for stage 1 non-squamous NSCLC. (A,B) Kaplan-Meier survival analysis of the test set from the microarray meta-cohort with stratification of risk groups based on the median value of the CRM. (C,D) Kaplan-Meier survival analysis of the validation qPCR cohort with risk groups defined by the median CRM value in the training set.

FIG. 5: Robust identification of prognostic genes requires sample sizes of >350. Black=hazard ratio; blue=z-score; red and green=upper/lower 95% CI on hazard ratio.

FIG. 6: Histology of a stage 1 lung adenocarcinoma. Malignant glands are depicted in hematoxylin- and eosin-stained sections. (A) An overlay of light blue and light brown delineate stromal and epithelial portions of the tissue, respectively. The sections were also stained by IHC for TTF-1 (A,B), Ki67 (C), and CD20 (D). Mki67, which encodes the Ki67 protein, and Ms4a1, which encodes the CD20 protein, both are significantly associated with OS in stage 1 non-squamous NSCLC. Arrowheads denote examples of cells stained by IHC.

FIG. 7: (A) Slc2a1 RNA levels in early stage non-squamous NSCLC, as determined by TaqMan qRT-PCR, is highly correlated with GLUT1 staining by IHC (N=90 cases). (B) Slc2a1 RNA levels correlate with tumor SUVmax on PET scans performed as part of routine clinical care (N=53 cases).

### DETAILED DESCRIPTION OF THE EMBODIMENTS

Methods, kits, devices, and computer systems are provided for obtaining an NSCLC marker level representation for an individual with non small cell lung carcinoma (NSCLC); and/or for providing a prognosis for an individual with NSCLC. The methods can include measuring expression levels, in a biological sample, of 2 or more NSCLC markers selected from: MAD2L1, GINS1, SLC2A1, KRT6A, FCGRT, TNIK, BCAM, KDM6A, and FAIM3; calculating an NSCLC marker level representation based on the measured expression levels; comparing the NSCLC marker level representation of the individual to a reference marker level representation; providing a prognosis based on the comparison; and/or generating a report that includes at least one of: (i) an NSCLC marker level representation, (ii) an NSCLC marker level representation and a reference NSCLC marker level representation, (iii) a prognosis, and (iv) guidance to a clinician as to a treatment recommendation based on the prognosis.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### Methods

As summarized above, aspects of the disclosure include methods for obtaining an NSCLC marker level representation for an individual with non small cell lung carcinoma (NSCLC); and/or for providing a prognosis for an individual with NSCLC.

Non Small Cell Lung Carcinoma (NSCLC).

About 85% to 90% of lung cancers are non small cell lung carcinoma (NSCLC), which can lead to death. There are 3 main subtypes of NSCLC: (1) Squamous cell (epidermoid) carcinoma: These cancers start in early versions of squamous cells, which are flat cells that line the inside of the airways in the lungs. They are often linked to a history of smoking and tend to be found in the middle of the lungs, near a bronchus; (2) Adenocarcinoma: These cancers start in early versions of the cells that would normally secrete substances such as mucus. Adenocarcinoma is usually found in outer parts of the lung. It tends to grow slower than other types of lung cancer, and is more likely to be found before it has spread outside of the lung; and (3) Large cell (undifferentiated) carcinoma: This type of cancer can appear in any part of the lung. It tends to grow and spread quickly, which can make it harder to treat. A subtype of large cell carcinoma, known as large cell neuroendocrine carcinoma, is a fast-growing cancer that is very similar to small cell lung cancer.

In some embodiments, the NSCLC can be a stage 1 NSCLC, a non-squamous NSCLC, or a stage 1 non-squamous NSCLC. Staging of lung cancer will be known to one of ordinary skill in the art. For example, information about staging lung cancer (including NSCLC) can be found in: Lung. In: Edge S B, Byrd D R, Compton C C, et al., eds.: AJCC Cancer Staging Manual. 7th ed. New York, N.Y.: Springer, 2010, pp 253-70, which is hereby incorporated by reference in its entirety. In some cases, staging can be according to AJCC Cancer Staging Manual. 6th edition. Additional information can also be found online at the website for the National Cancer Institute at “www” followed by “.cancer.gov”.

NSCLC Marker Level Representation.

By a “NSCLC marker level representation”, it is meant a representation of the levels of two or more of the subject NSCLC marker(s), e.g. a panel of NSCLC markers, in a biological sample from an individual. In some aspects of the disclosure, NSCLC markers and panels of NSCLC markers are provided. By an “NSCLC marker” it is meant a molecular entity (e.g., mRNA, protein) whose representation in a sample correlates (either positively or negatively) with an NSCLC phenotype (e.g., a particular likelihood of survival, e.g., high likelihood (low risk category), low likelihood (high risk category), etc.). For example, an NSCLC marker may be differentially represented, i.e. represented at a different level, in a sample from an individual with NSCLC that has a high likelihood of survival (e.g., low risk category) relative to a reference (e.g., an individual with a low likelihood of survival (e.g., high risk category); a population of individuals with NSCLC having individuals with various likelihoods of survival; and the like).

As demonstrated in the examples of the present disclosure, the inventors have identified a number of molecular entities (markers) that are associated with increased likelihood of survival in patients with NSCLC, and that find use either alone or in combination (i.e. as a panel) in providing an NSCLC assessment, e.g. a prognosis, a recommended treatment regimen, and the like. The identified markers include, but are not limited to:

Note: “NM” sequences are cDNA sequences of mRNA transcripts; and
“NP” sequences are protein sequences
1. BCAM (basal cell adhesion molecule (Lutheran blood group)): Isoform 1 (NM_005581.4; NP_005572.2); Isoform 2 (NM_001013257.2; NP_001013275.1) (SEQ ID NOs: 1-4).
2. FAIM3 (Fas apoptotic inhibitory molecule 3): Isoform a (NM_005449.4; NP_005440.1); Isoform b (NM_001142473.1; NP_001135945.1); Isoform c (NM_001193338.1; NP_001180267.1) (SEQ ID NOs: 5-10).
3. FCGRT (Fc fragment of IgG, receptor, transporter, alpha): Variant 1 (NM_001136019.2; NP_001129491.1); Variant 2 (NM_004107.4; NP_004098.1) (SEQ ID NOs: 11-14).
4. GINS1 (GINS complex subunit 1 (Psf1 homolog)): (NM_021067.3; NP_066545.3) (SEQ ID NOs: 17-18).
5. KDM6A (lysine (K)-specific demethylase 6A): (NM_021140.2; NP_066963.2) (SEQ ID NOs: 19-20).
6. KRT6A (keratin 6A): (NM_005554.3; NP_005545.1) (SEQ ID NOs: 21-22).
7. SLC2A1 (solute carrier family 2 (facilitated glucose transporter), member 1): NM_006516.2; NP_006507.2) (SEQ ID NOs: 23-24).
8. TNIK (TRAF2 and NCK interacting kinase): Isoform 1 (NM_015028.2; NP_055843.1); Isoform 2 (NM_001161560.1; NP_001155032.1); Isoform 3 (NM_001161561.1; NP_001155033.1); Isoform 4 (NM_001161562.1; NP_001155034.1); Isoform 5 (NM_001161563.1; NP_001155035.1); Isoform 6 (NM_001161564.1; NP_001155036.1); Isoform 7 (NM_001161565.1; NP_001155037.1); Isoform 8 (NM_001161566.1; NP_001155038.1) (SEQ ID NOs: 25-40).
9. MAD2L1 (MAD2 mitotic arrest deficient-like 1 (yeast)): (NM_002358.3; NP_002349.1) (SEQ ID NOs: 41-42).
10. FSCN1 (fascin homolog 1, actin-bundling protein (Strongylocentrotus purpuratus)): (NM_003088.3; NP_003079.1) (SEQ ID NOs: 15-16).

The level of any combination of the above biomarkers can be measured and utilized in the subject methods. In some instances, an elevated level of marker (a “positive biomarker”) is associated with an increased likelihood of survival (e.g., low risk). In other words, the expression levels of positive biomarkers correlate positively with a positive prognosis (correlate negatively with a negative prognosis). For example, the expression level (e.g., the number of transcripts, the concentration in a sample, and the like) of a positive biomarker may be 1.5-fold, 2-fold, 2.5-fold, 3-fold, 4-fold, 5-fold, 7.5-fold, 10-fold, or greater in a sample associated with increased likelihood of survival (e.g., low risk) compared to a sample not associated with increased likelihood of survival (e.g., a reference, e.g., an intermediate risk reference, a high risk reference, etc.). In some cases, the expression level of a positive biomarker may be reduced by 10% or more, 20% or more, 30% or more, 40% or more, 50% or more, 60% or more, 70% or more, 80% or more or 90% or more in a sample associated with decreased likelihood of survival (e.g., high risk) compared to a sample not associated with decreased likelihood of survival (e.g., a reference, e.g., an intermediate risk reference, a low risk reference, etc.). In some cases, the expression level of a positive biomarker may be decreased by about 1.5-fold or more (e.g., 2-fold or more, 2.5-fold or more, 3-fold or more, 3.5-fold or more, 4-fold or more, 4.5-fold or more, or 5-fold or more, 8-fold or more, 10-fold or more, 15-fold or more) in a sample associated with decreased likelihood of survival (e.g., high risk) compared to a sample not associated with decreased likelihood of survival (e.g., a reference, e.g., an intermediate risk reference, a low risk reference, etc.). Positive biomarkers include, but are not necessarily limited to: FCGRT, TNIK, BCAM, KDM6A, and FAIM3.

In other instances, a reduced level of marker (a “negative biomarker”) is associated with the NSCLC phenotype. In other words, the expression levels of negative biomarkers correlate negatively with a positive prognosis (correlate positively with a negative prognosis). For example, the expression level (e.g., the number of transcripts, the concentration in a sample, and the like) of a negative biomarker may be reduced by 10% or more, 20% or more, 30% or more, 40% or more, 50% or more, 60% or more, 70% or more, 80% or more or 90% or more in a sample associated with increased likelihood of survival (e.g., low risk) compared to a sample not associated with increased likelihood of survival (e.g., a reference, e.g., an intermediate risk reference, a high risk reference, etc.). In some cases, the expression level of a negative biomarker may be decreased by about 1.5-fold or more (e.g., 2-fold or more, 2.5-fold or more, 3-fold or more, 3.5-fold or more, 4-fold or more, 4.5-fold or more, or 5-fold or more, 8-fold or more, 10-fold or more, 15-fold or more) in a sample associated with increased likelihood of survival (e.g., low risk) compared to a sample not associated with increased likelihood of survival (e.g., a reference, e.g., an intermediate risk reference, a high risk reference, etc.). In some cases, the expression level of a negative biomarker may be 1.5-fold, 2-fold, 2.5-fold, 3-fold, 4-fold, 5-fold, 7.5-fold, 10-fold, or greater in a sample associated with decreased likelihood of survival (e.g., high risk) compared to a sample not associated with decreased likelihood of survival (e.g., a reference, e.g., an intermediate risk reference, a low risk reference, etc.). Negative biomarkers include, but are not necessarily limited to: MAD2L1, GINS1, SLC2A1, and KRT6A.

Also provided herein are NSCLC panels. By a “panel” of NSCLC markers it is meant two or more NSCLC markers (e.g. 3 or more, 4 or more, 5 or more, 6 or more, 7 or more, 8 or more, 9 or more, or 10 or more markers, e.g. 2 markers, 3 markers, 4 markers, 5 markers, 6 markers, 7 markers, 8 markers, 9 markers, 10 markers, etc.) whose levels, when considered in combination, find use in providing a NSCLC assessment, e.g. making a NSCLC diagnosis, prognosis, treatment recommendation, treatment prescription, and the like. In some embodiments, a subject NSCLC panel may include: (i) MAD2L1, GINS1, SLC2A1, and KRT6A; (ii) FCGRT, TNIK, BCAM, KDM6A, and FAIM3; (iii) MAD2L1, GINS1, SLC2A1, KRT6A, FCGRT, TNIK, BCAM, KDM6A, and FAIM3; (iv) MAD2L1, GINS1, SLC2A1, KRT6A, FCGRT, TNIK, BCAM, KDM6A, FAIM3, and FSCN1; and any combination thereof. For example, an NSCLC panel can include any combination of positive and negative biomarkers (e.g., can include only positive biomarkers, can include only negative biomarkers, can include a combination of positive and negative biomarkers, etc.). In some cases, a subject method includes measuring expression levels, in a biological sample, of 2 or more NSCLC markers (e.g., 3 or more, 4 or more, 5 or more, 6 or more, 7 or more, 8 or more, etc.) selected from: MAD2L1, GINS1, SLC2A1, KRT6A, FCGRT, TNIK, BCAM, KDM6A, and FAIM3. In some cases, a subject method includes measuring expression levels, in a biological sample, of the nine NSCLC markers: MAD2L1, GINS1, SLC2A1, KRT6A, FCGRT, TNIK, BCAM, KDM6A, and FAIM3.

In some cases, a subject method includes measuring expression levels, in a biological sample, of 2 or more NSCLC markers (e.g., 3 or more, 4 or more, 5 or more, 6 or more, 7 or more, 8 or more, 9 or more, etc.) selected from: MAD2L1, GINS1, SLC2A1, KRT6A, FCGRT, TNIK, BCAM, KDM6A, FAIM3, and FSCN1. In some cases, a subject method includes measuring expression levels, in a biological sample, of the ten NSCLC markers: MAD2L1, GINS1, SLC2A1, KRT6A, FCGRT, TNIK, BCAM, KDM6A, FAIM3, and FSCN1. Other combinations of NSCLC markers that find use as NSCLC panels in the subject methods may be identified by the ordinarily skilled artisan using any convenient statistical methodology, e.g. as described in the working examples herein.

### Measuring Expression Levels

The term “assaying” is used herein to include the physical steps of manipulating a biological sample to generate data related to the sample (e.g., measuring an expression level). As will be readily understood by one of ordinary skill in the art, a biological sample must be “obtained” prior to assaying the sample. Thus, the terms “assaying” and “measuring” implies that the sample has been obtained. The terms “obtained” or “obtaining” as used herein encompass the physical extraction or isolation of a biological sample from a subject. The terms “obtained” or “obtaining” as used herein also encompasses the act of receiving an extracted or isolated biological sample. For example, a testing facility can “obtain” a biological sample in the mail (or via delivery, etc.) prior to assaying the sample. In some such cases, the biological sample was “extracted” or “isolated” (and thus “obtained”) from the subject by a second entity prior to mailing, and then “obtained” by the testing facility upon arrival of the sample. Thus, the testing facility can obtain the sample and then assay the sample (e.g., measure expression levels from the sample), thereby producing data related to the sample. Alternatively, a biological sample can be extracted or isolated from a subject by the same person or same entity that subsequently assays the sample. In some embodiments, a subject method includes: obtaining a biological sample and measuring the expressional levels of 2 or more NSCLC markers in the sample.

In practicing the subject methods, the level(s) of NSCLC marker(s) in the biological sample from an individual are measured. The level of one or more NSCLC markers in the subject sample may be measured by any convenient method. For example, NSCLC gene expression levels may be detected by measuring the levels/amounts of one or more nucleic acid transcripts, e.g. mRNAs, of one or more NSCLC genes. Protein markers may be detected by measuring the levels/amounts of one or more proteins/polypeptides.

The terms “measuring” and “analyzing” are used herein to refer to any form of measurement, and include determining if an element is present or not. These terms include both quantitative and/or qualitative determinations. Assaying may be relative or absolute. For example, “measuring” can be determining whether the expression level is less than or “greater than or equal to” a particular threshold, (the threshold can be predetermined or can be determined by assaying a control sample). On the other hand, “measuring to determine the expression level” or simply “measuring expression levels” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI), threshold cycle (Cₜ), quantification cycle (C_{q}), and the like), or can be expressed as an absolute value with defined units (e.g., number of mRNA transcripts, number of protein molecules, concentration of protein, etc.).

The level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple markers and/or multiple biological samples (e.g., samples from multiple individuals or multiple samples from the same individual).

NSCLC markers may include proteins associated with an NSCLC prognosis (e.g., positive and/or negative biomarkers, as discussed above) and their corresponding genetic sequences, i.e. mRNA, DNA, etc. By a “gene” or “recombinant gene” it is meant a nucleic acid comprising an open reading frame that encodes for the protein. The boundaries of a coding sequence are determined by a start codon at the 5′ (amino) terminus and a translation stop codon at the 3′ (carboxy) terminus. A transcription termination sequence may be located 3′ to the coding sequence. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site (e.g., 5′ UTR), and may or may not include untranslated leader sequences, signal sequences, downstream untranslated sequences (e.g., 3′ UTR), transcriptional start and stop sequences, polyadenylation signals, translational start and stop sequences, ribosome binding sites, and the like. When referring to an NSCLC marker herein, it is meant any nucleic acid and/or amino acid sequence that can be identified that is uniquely associated with the corresponding gene. For example, if the 5′UTR of an NSCLC associated gene (an NSCLC biomarker) contains a first sequence that is unique to that particular gene, then the associated biomarker can be a sequence that includes that first unique sequence.

### Measuring RNA

The level of at least one NSCLC marker may be evaluated by detecting in a patient sample (a biological sample from an individual with NSCLC) the amount or level of one or more RNA transcripts or a fragment thereof encoded by the gene of interest. For measuring RNA levels, the amount or level of an RNA in the sample is determined, e.g., the expression level of an mRNA. In some instances, the expression level of one or more additional RNAs may also be measured, and the level of biomarker expression compared to the level of the one or more additional RNAs to provide a normalized value for the biomarker expression level.

The expression level of nucleic acids in the sample may be detected using any convenient protocol. A number of exemplary methods for measuring RNA (e.g., mRNA) expression levels (e.g., expression level of a nucleic acid biomarker) in a sample are known by one of ordinary skill in the art, such as those methods employed in the field of differential gene expression analysis, and any convenient method can be used. Exemplary methods include, but are not limited to: hybridization-based methods (e.g., Northern blotting, array hybridization (e.g., microarray); in situ hybridization; in situ hybridization followed by FACS; and the like) (Parker & Barnes, Methods in Molecular Biology 106:247-283 (1999)); RNAse protection assays (Hod, Biotechniques 13:852-854 (1992)); PCR-based methods (e.g., reverse transcription PCR (RT-PCR), quantitative RT-PCR (qRT-PCR), real-time RT-PCR, etc.) (Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLID platform), Life Technologies' Ion Torrent platform, single molecule sequencing, etc.); nanopore based sequencing methods; and the like.

In some embodiments, the biological sample can be assayed directly. In some embodiments, nucleic acid of the biological sample is amplified (e.g., by PCR) prior to assaying. As such, techniques such as PCR (Polymerase Chain Reaction), RT-PCR (reverse transcriptase PCR), qRT-PCR (quantitative RT-PCR, real time RT-PCR), etc. can be used prior to the hybridization methods and/or the sequencing methods discussed above.

As noted above, gene expression in a sample can be detected using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative. Pattern analysis can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, quantitative RT-PCR (qRT-PCR), and the like, e.g. TaqMan® RT-PCR, SYBR green; MassARRAY® System, BeadArray® technology, and Luminex technology; and those that rely upon hybridization of probes to filters, e.g. Northern blotting and in situ hybridization. Other non-amplified methods of analysis include digital bar-coding, e.g. NanoString nCounter Analysis System which is a digital color-coded barcode technolog based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest. Mixed together with controls, they form a multiplexed CodeSet.

Examples of some of the nucleic acid sequencing methods listed above are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39); Soni et al Clin Chem 53: 1996-2001 2007; and Morozova (Genomics. 2008 92:255-64), which are incorporated by reference for the general descriptions of the methods and the particular steps of the methods, including starting products, reagents, and final products for each of the steps.

For measuring mRNA levels, the starting material is typically total RNA or poly A+ RNA isolated from a biological sample (e.g., suspension of cells from a peripheral blood sample, an aspirate, a formalin-fixed paraffin embedded (FFPE) tissue sample, a biopsy sample, an FFPE biopsy sample, etc., or from a homogenized tissue, e.g. a homogenized biopsy sample, a homogenized paraffin- or OCT-embedded sample, etc.). General methods for mRNA extraction are known in the art and are disclosed in standard textbooks of molecular biology, including Ausubel et al., Current Protocols of Molecular Biology, John Wiley and Sons (1997). RNA isolation (e.g., mRNA isolation) can be performed using any convenient protocol. For example, RNA isolation can be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

### Measuring Protein

The level of at least one NSCLC marker may be measured by detecting in a patient sample (a biological sample from an individual with NSCLC) the amount or level of one or more proteins or a fragment thereof encoded. For measuring protein levels, the amount or level of a polypeptide in the biological sample is determined. In some instances, the concentration of one or more additional proteins may also be measured, and biomarker concentration compared to the level of the one or more additional proteins to provide a normalized value for the biomarker concentration. In some embodiments concentration is a relative value measured by comparing the level of one protein relative to another protein. In other embodiments the concentration is an absolute measurement of weight/volume or weight/weight.

The level of at least one NSCLC marker may be measured by detecting in a sample the amount or level of one or more proteins/polypeptides or fragments thereof to arrive at a protein level representation. The terms “polypeptide,” “peptide” and “protein” are used interchangeably herein to refer to a polymer of amino acid residues. “Polypeptide” refers to a polymer of amino acids (amino acid sequence) and does not refer to a specific length of the molecule. Thus peptides and oligopeptides are included within the definition of polypeptide. This term also refers to or includes post-translationally modified polypeptides, for example, glycosylated polypeptide, acetylated polypeptide, phosphorylated polypeptide and the like. Included within the definition are, for example, polypeptides containing one or more analogs of an amino acid, polypeptides with substituted linkages, as well as other modifications known in the art, both naturally occurring and non-naturally occurring.

In some embodiments, the extracellular protein level is measured. For example, in some cases, the protein (i.e., polypeptide) being measured is a secreted protein (e.g., a cytokine or chemokine) and the concentration can therefore be measured in the extracellular fluid of a biological sample (e.g., the concentration of a protein can be measured in the serum, in an aspirate of the lungs, in fluid surrounding a biopsy, in extracellular fluid from a biopsy, etc.). In some cases, the cells are removed from the biological sample (e.g., via centrifugation, via adhering cells to a dish or to plastic, etc.) prior to measuring the concentration. In some cases, the intracellular protein level is measured by lysing cells of the biological sample (e.g., a biopsy) to measure the level of protein in the cellular contents. In some cases, both the extracellular and cell-associated levels of protein are measured by separating the cellular and fluid portions of the biological sample (e.g., via centrifugation), measuring the extracellular level of the protein by measuring the level of protein in the fluid portion of the biological sample, and measuring the cell-associated level of protein by measuring the level of protein in the cell-associated portion of the biological sample (e.g., after lysing the cells). In some cases, the total level of protein (i.e., combined extracellular and cell-associated protein) is measured by lysing the cells of the biological sample to include the cell-associated protein contents as part of the sample.

When protein levels are to be detected, any convenient protocol for measuring protein levels may be employed. For example, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, flow cytometry, western blotting, and immunohistochemistry.

Some protein detection methods are antibody-based methods. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The term “specific binding agent” as used herein refers to any agent that specifically binds a biomolecule (e.g., a marker such as a nucleic acid marker molecule, a protein marker molecule, etc.). In some cases, a “specific binding agent” for a marker molecule (e.g., a biomarker) is used. Specific binding agents can be any type of molecule. In some cases, a specific binding agent is an antibody or a fragment thereof. In some cases, a specific binding agent is nucleic acid probe (e.g., an RNA probe; a DNA probe; an RNA/DNA probe; a modified nucleic acid probe, e.g., a locked nucleic acid (LNA) probe, a morpholino probe, etc.; and the like).

Biological Samples.

Aspects of the disclosure include measuring expression levels in a biological sample from the individual. The terms “recipient”, “individual”, “subject”, “host”, and “patient”, are used interchangeably herein and refer to any mammalian subject for whom measurement of expression levels, prognosis, diagnosis, treatment, and/or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples (e.g., a biopsy). Clinical samples for use in the methods of the invention may be obtained from a variety of sources including, but not limited to a biopsy sample (e.g., a lung biopsy sample), a thoracentesis sample, a fine needle aspirate, and the like. Exemplary biological samples include, but are not limited to: a suspension of cells (e.g., from a peripheral blood sample, an aspirate, a cell suspension from a biopsy sample, etc.), a biopsy, an aspirate (e.g., a fine needle aspirate, a thoracentesis sample, etc.), a fixed tissue sample (e.g., a formalin-fixed paraffin embedded (FFPE) tissue sample, an FFPE biopsy sample, etc.), and a homogenized tissue (e.g., a homogenized biopsy sample, a homogenized paraffin- or OCT-embedded sample, etc.).

Once a sample is isolated (i.e., collected), it can be used directly, frozen, or maintained in appropriate culture medium for a period of time (e.g., in some cases, an extended period of time). Typically the samples will be from human patients, although animal models may find use, e.g. equine, bovine, porcine, canine, feline, rodent, e.g. mice, rats, hamster, primate, etc. Any convenient tissue sample that demonstrates differential representation of the one or more NSCLC markers disclosed herein among NSCLC patients with varying likelihoods of survival may be evaluated in the subject methods. Typically, a suitable sample source can be derived from a tumor (e.g., a biopsy) and/or fluids associated with a tumor.

The subject sample may be treated in a variety of ways so as to enhance detection of the one or more NSCLC markers. For example, where the sample is a tumor sample (e.g., a biopsy), non-tumor cells may be removed from the sample (e.g., by differential centrifugation, by differential binding and/or labeling, e.g., FACs sorting and/or magnetic separation techniques) prior to assaying Where the sample is blood, the red blood cells may be removed from the sample (e.g., by centrifugation) prior to assaying. Such a treatment may serve to reduce the non-specific background levels of detecting the level of a NSCLC marker using an affinity reagent. Detection of a NSCLC marker may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment can prevent complex formation, thereby providing a more accurate quantitation of the level of marker in the sample. In embodiments where the sample is urine, the pH of the sample can be adjusted and the sample can be concentrated in order to enhance the detection of the marker.

Calculating an NSCLC Marker Level Representation.

Once a value for the expression level of the one or more NSCLC markers has been determined, the measurement(s) may be analyzed in a number of ways to obtain a NSCLC marker level representation. For example, the measurements of 2 or more NSCLC markers may be analyzed individually to develop an NSCLC marker expression profile. As used herein, an “NSCLC marker expression profile” is the normalized levels of 2 or more NSCLC markers in a patient sample, for example, the normalized levels of MAD2L1, GINS1, SLC2A1, KRT6A, FCGRT, TNIK, BCAM, KDM6A, FAIM3, and/or FSCN1 in a patient sample. A profile may be generated by any of a number of methods known in the art. For example, the level of each marker may be log₂ transformed and/or normalized (e.g., relative to the expression of one or more housekeeping genes such as AGPAT1, PRPF40A, ABL1, GAPDH, PGK1, ACTB, RPLPO, GUS, TFRC, HPRT1, ESD, GUSB, HMBS, B2M, IPO8, PPIA, PGK1, RPS11, RPL0, RPL10, RPL14, RPL18, BAT1, TBP, and the like; relative to the signal across a whole panel, e.g., relative to the overall number of “reads” in a sample, etc). As described in more detail below, the expression levels of a NSCLC expression profile can also be weighted. Other methods of calculating a NSCLC profile will be readily known to the ordinarily skilled artisan.

The resultant data (from measuring the expression levels, RNA and/or protein, of NSCLC markers) provides information regarding levels in the sample for each of the markers that have been probed, wherein the information is in terms of whether or not the marker is present and, typically, at what level, and wherein the data may be qualitative and/or quantitative. As such, where detection is qualitative, the methods provide a reading or evaluation, e.g., assessment, of whether or not the target marker, e.g., nucleic acid or protein, is present in the sample being assayed. In yet other embodiments, the methods provide a quantitative detection of whether the target marker is present in the sample being assayed, i.e., an evaluation or assessment of the actual amount or relative abundance of the target analyte, e.g., nucleic acid or protein in the sample being assayed. In such embodiments, the quantitative detection may be absolute or relative (e.g., if the method is a method of detecting two or more different analytes, e.g., target nucleic acids or proteins, in a sample). As such, the term “quantifying” when used in the context of quantifying a target analyte, e.g., nucleic acid(s) or protein(s), in a sample can refer to absolute or to relative quantification. Absolute quantification may be accomplished by inclusion of known concentration(s) of one or more control analytes and referencing the detected level of the target analyte with the known control analytes (e.g., through generation of a standard curve).

Relative quantification (also called normalization) can be accomplished by comparison of detected levels or amounts between two or more different target analytes to provide a relative quantification of each of the two or more different analytes, e.g., relative to each other. In some cases, normalization can be accomplished by comparison of detected levels of an analyte followed by normalization. For example, in cases where a nucleic acid analyte is quantified by counting (e.g., counting the number of “reads” that map to (i.e., can be assigned to) the analyte of interest when performing high throughput sequencing methods), the number of “reads” and/or “fragments” counted for the target analyte can be normalized to the number of overall reads in the sample and/or can be normalized for the length of the target nucleic acid (this type of normalization typically results in reads per thousand bases per million reads (RPKM) or fragments per thousand bases per million reads (FPKM) as is known in the art). Any convenient means of normalization can be performed. As non-limiting examples, normalization techniques can include: using algorithms such as the MAS5 algorithm (see, e.g, Pepper et al, BMC Bioinformatics 2007, 8:273), quantile normalization, and/or Robust Multi-array Average (RMA).

As another example of using measured expression levels of the two or more NSCLC markers to obtain an NSCLC marker level representation, the measurements of a panel of NSCLC markers (e.g., two or more NSCLC markers) may be analyzed collectively to arrive at (i.e., calculate) a single NSCLC score. By an “NSCLC score” it is meant a single metric value (e.g., a molecular prognostic index (MPI)) that represents a combination of the expression levels of the NSCLC markers in the NSCLC panel. Calculating an NSCLC score can include normalizing the measured expression levels. Calculating an NSCLC score can include weighting the measured expression levels. Calculating an NSCLC score can include normalizing and weighting the expression levels (e.g., normalizing the weighted expression levels, weighting the normalized expression levels, etc.).

As such, in some embodiments, the subject method comprises measuring the expression levels of markers of an NSCLC panel in the sample, and calculating a NSCLC score (e.g., based on the normalized and/or weighted expression levels of the NSCLC markers). A NSCLC score for a patient sample may be calculated by any convenient method and/or algorithm for calculating biomarker scores. For example, weighted marker levels, e.g. log₂ transformed and normalized marker levels that have been weighted by, e.g., multiplying each normalized marker level to a weighting factor, may be totaled and in some cases averaged to arrive at a single value representative of the panel of NSCLC markers analyzed.

In some instances, the weighting factor, or simply “weight” for each marker in a panel may be a reflection of the change in analyte level in the sample. For example, the analyte level of each NSCLC marker may be log₂ transformed and weighted either as 1 (for those markers that exhibit a relative increase) or −1 (for those markers that exhibit a relative decrease), and the sum of increased markers and decreased markers determined to arrive at a NSCLC score. In other instances, the weights may be reflective of the importance of each marker to the specificity, sensitivity and/or accuracy of the marker panel in making the diagnostic, prognostic, or monitoring assessment. Such weights may be determined by any convenient method, e.g., statistical machine learning methodology, e.g. Principal Component Analysis (PCA), linear regression, support vector machines (SVMs), and/or random forests of the dataset from which the sample was obtained may be used. In some instances, weights for each marker are defined by the dataset from which the patient sample was obtained. In other instances, weights for each marker may be defined based on a reference dataset, or “training dataset”. Any dataset relating to patients having NSCLC may be used as a reference dataset. For example, the weights may be determined based upon any of the datasets and/or results provided in the examples section below.

As an example, in some embodiments, the NSCLC marker level representation is provided as an NSCLC score (e.g., an MPI value), which is a single metric value that represents the sum of the weighted expression levels of 2 or more markers in a patient sample. An MPI value can be determined by methods very similar to those described above, e.g. the expression levels of each of the 2 or more genes in a patient sample may be log₂ transformed and normalized; the normalized expression levels for each gene can then weighted by multiplying the normalized level to a weighting factor, or “weight”, to arrive at weighted expression levels for each of the one or more genes; and the weighted expression levels can then be totaled and in some cases averaged to arrive at a single weighted expression level for the genes analyzed. This analysis may be readily performed by one of ordinary skill in the art by employing a computer-based system, e.g. using any hardware, software and data storage medium as is known in the art, and employing any algorithms convenient for such analysis.

As an example of weighting expression levels, in some cases, in an NSCLC panel comprising the markers MAD2L1, GINS1, SLC2A1, KRT6A, FCGRT, TNIK, BCAM, KDM6A, and FAIM3: FAIM3 levels are most significant, levels of KDM6A, BCAM, TNIK, FCGRT, and SLC2A1 are moderately important, and levels of MAD2L1, GINS1, and KRT6A are less significant. As such, one example of an algorithm that may be used to arrive at a NSCLC score would be an algorithm that considers FAIM3 levels most strongly (e.g. assigning FAIM3 measurements a weight of 5-7, e.g. 6); that considers KDM6A, BCAM, TNIK, FCGRT, and SLC2A1 levels more modestly (e.g. assigning the measurements for these genes a weight of 4-6, e.g. 5); and that considers MAD2L1, GINS1, and KRT6A levels least (e.g. assigning MAD2L1, GINS1, and KRT6A measurements a weight of 3-5, e.g, 4).

In some embodiments (e.g., when calculating an NSCLC score includes calculating an MPI value), calculating an MPI value includes weighting the expression levels of the 2 or more NSCLC markers such that the expression levels of MAD2L1, GINS1, and KRT6A are each multiplied by 4 (4 is therefore considered the weighting coefficient for MAD2L1, GINS1, and KRT6A); the expression level of SLC2A1 is multiplied by 5; the expression levels of FCGRT, TNIK, BCAM, and KDM6A are each multiplied by −5; and/or the expression level of FAIM3 is multiplied by −6. The above weighting coefficients for each marker represent the weighted expression values relative to one other. As such, it will be readily appreciated by one of ordinary skill in the art that the weighting coefficient can by multiplied by any factor as long as all coefficients in the formula are equally multiplied (for those genes being used to calculate the score). For example, the weighting coefficients for MAD2L1, GINS1, and KRT6A can be 0.04, 0.4, 4, etc. as long as the weighting coefficient for SLC2A1 is 0.05, 0.5, 5, etc., respectively; the weighting coefficients for FCGRT, TNIK, BCAM, and KDM6A are −0.05, −0.5, −5, etc., respectively; and the weighting coefficient for FAIM3 is −0.06, −0.6, −6, etc., respectively.

In some embodiments calculating an MPI value includes adding, subtracting, and weighting the expression levels according to the formula:

4y₁+4y₂+4y₃+5y₄−5y₅−5y₆−5y₇−5y₈−6y₉ Formula (I)

where each of y₁ to y₉ are the expression levels (e.g., normalized expression levels) of the corresponding NSCLC marker, and wherein: (i) y₁, y₂, and y₃ are MAD2L1, GINS1, and KRT6A; (ii) y₄ is SLC2A1; (iii) y₅, y₆, y₇, and y₈ are FCGRT, TNIK, BCAM, and KDM6A; and (iv) y₉ is FAIM3. As noted above, the weighting coefficients for each marker represent the weighted expression values relative to one other. As such, it will be readily appreciated by one of ordinary skill in the art that the weighting coefficient can by multiplied by any factor as long as all coefficients in the formula are equally multiplied (for those genes being used to calculate the score). For example, calculating an MPI value can include adding, subtracting, and weighting the expression levels according to the formula: 0.04y₁+0.04y₂+0.04y₃+0.05y₄−0.05y₅−0.05y₆−0.05y₇−0.05y₈−0.06y₉; according to the formula 0.4y₁+0.4y₂+0.4y₃+0.5y₄−0.5y₅−0.5y₆−0.5y₇−0.5y₈−0.6y₉; etc.

By way of illustration, the example calculation and formula above assigned negative values to positive biomarkers and positive values to negative biomarkers, and thus, the NSCLC score would correlate positively with a negative outcome (e.g., increasing NSCLC scores would indicate increasing risk/lower survival likelihood while decreasing NSCLC scores would indicate decreasing risk/higher survival likelihood). Likewise, the calculation and formula above could be written such that positive biomarkers are assigned positive values and negative biomarkers are assigned negative values, which would mean that the NSCLC score would correlate positively with a positive outcome (e.g., increasing NSCLC scores would indicate decreasing risk/higher survival likelihood while decreasing NSCLC scores would indicate increasing risk/lower survival likelihood).

In some cases, if an individual's calculated NSCLC score (e.g., MPI, CRM) is below the threshold, the individual has a decreased risk (e.g., increased likelihood of survival) relative to someone whose calculated NSCLC score is above the threshold. In some cases, if an individual's calculated NSCLC score is below the threshold, the individual can be said to have “low risk” (high likelihood of survival), and if an individual's calculated NSCLC score is above the threshold, the individual can be said to have “high risk” (low likelihood of survival). In some cases (e.g., see working examples below)(e.g., when the weighting coefficient for MAD2L1, GINS1, and KRT6A is 0.04; the weighting coefficient for SLC2A1 is 0.05; the weighting coefficient for FCGRT, TNIK, BCAM, and KDM6A is −0.05; and the weighting coefficient for FAIM3 is −0.06), the threshold can be in a range of from 1.5 to 3.5 (e.g., from 1.6 to 3.4, from 1.7 to 3.3, from 1.8 to 3.2, from 1.9 to 3.1, from 2.0 to 3.0, from 2.1 to 2.9, from 2.1 to 2.8, from 2.1 to 2.7, from 2.2 to 2.6, from 2.3 to 2.5, from 2.1 to 2.3, from 2.5 to 2.7, from 2.7 to 2.9, from 2.9 to 3.1, from 2.0 to 2.2, from 2.2 to 2.4, from 2.4 to 2.6, from 2.6 to 2.8, from 2.8 to 3.0, 2.0, 2.1, 2.2, 2.3, 2.4, 2.5, 2.6, 2.7, 2.8, 2.9, or 3.0). In some such cases, the threshold is 2.4. The threshold numbers can also be multiplied as discussed above by a factor relative to the rest of the terms in the equation. For example, in some cases (e.g., when the weighting coefficient for MAD2L1, GINS1, and KRT6A is 4; the weighting coefficient for SLC2A1 is 5; the weighting coefficient for FCGRT, TNIK, BCAM, and KDM6A is −5; and the weighting coefficient for FAIM3 is −6), the threshold can be in a range of from 150 to 350 (e.g., from 160 to 340, from 170 to 330, from 180 to 320, from 190 to 310, from 200 to 300, from 210 to 290, from 210 to 280, from 210 to 270, from 220 to 260, from 230 to 250, from 210 to 230, from 250 to 270, from 270 to 290, from 290 to 310, from 200 to 220, from 220 to 240, from 240 to 260, from 260 to 280, from 280 to 300, 200, 210, 220, 230, 240, 250, 260, 270, 280, 290, or 300). In some such cases, the threshold is 240.

These methods of analysis may be readily performed by one of ordinary skill in the art by employing a computer-based system, e.g. using any hardware, software and data storage medium as is known in the art, and employing any algorithms convenient for such analysis. For example, data mining algorithms can be applied through “cloud computing”, smartphone based or client-server based platforms, and the like.

In certain embodiments the expression level (e.g. mRNA expression level, polypeptide level) of two markers is evaluated to produce a marker level representation. In yet other embodiments, the levels of two or more, i.e. a panel, markers, e.g., 3 or more, 4 or more, 5 or more, 6 or more, 7 or more, 8 or more, 9 or more, 10 or more, or 15 or more markers is evaluated. Accordingly, in the subject methods, the expression of at least two markers in a biological sample is evaluated. In certain embodiments, the evaluation that is made may be viewed as an evaluation of the expression level of the entire genome and/or proteome, as those terms are employed in the art.

In some instances, the subject methods of determining or obtaining an NSCLC marker level representation (e.g. an NSCLC profile and/or NSCLC score) for an individual further comprise providing the NSCLC marker representation as a report. Thus, in some instances, the subject methods may further include a step of generating or outputting a report providing the results of an NSCLC marker evaluation in the sample, which report can be provided in the form of a non-transient electronic medium (e.g., an electronic display on a computer monitor, stored in memory, etc.), or in the form of a tangible medium (e.g., a report printed on paper or other tangible medium). Any form of report may be provided, e.g. as known in the art or as described in greater detail below.

Integrating MPI with Clinical Variables

In some embodiments, the subject methods can include an assessment that is employed in conjunction with the subject marker level representation. For example, the subject methods may further comprise measuring one or more clinical parameters/factors associated with NSCLC, e.g. sex, stage (e.g., stage of NSCLC), grade (e.g., well-differentiated, moderately-differentiated, poorly-differentiated, undifferentiated, unknown, etc.), age, and whether surgical resection was performed (e.g., segmentectomy, wedge resection, lobectomy, partial pneumonectomy and total pneumonectomy). For example, a subject may be assessed for one or more clinical symptoms, which can be numerically codified, and which can be used in combination with the marker level representation to provide an NSCLC score.

In some instances, the clinical parameters may be measured prior to obtaining the NSCLC marker level representation, for example, to inform the artisan as to whether a NSCLC marker level representation should be obtained, e.g. to make or confirm an NSCLC prognosis. In some instances, the clinical parameters may be measured after obtaining the NSCLC marker level representation, e.g. to monitor an NSCLC.

An NSCLC score that is an integration of an MPI (discussed above) and a clinical score is herein referred to as a “composite risk model” (CRM). A clinical score can be integrated with an MPI by any convenient method. In some cases, a CRM is calculated by multiplying an MPI be a coefficient and adding the resultant value to weighted values based on one or more clinical scores. The weights (coefficients) to be used can be readily determined by one of ordinary skill in the art by analysis of a dataset (working examples are provided below) comprising survival data, NSCLC marker level representation data, and clinical data for individuals with NSCLC.

In some embodiments, for example when the weighting coefficient for MAD2L1, GINS1, and KRT6A is 4; the weighting coefficient for SLC2A1 is 5; the weighting coefficient for FCGRT, TNIK, BCAM, and KDM6A is −5; and the weighting coefficient for FAIM3 is −6; then CRM can be calculated according to the formula:

CRM=1.05*X+3.6Y+37Z Formula (II)

where X is the patient's calculated MPI; Y is the patient's age in years; and Z is a numerical description of the clinical score of the tumor stage (e.g, according to the TNM Classification of Malignant Tumors, which is a cancer staging system that describes the extent of a person's cancer and is known in the art, e.g., 1=stage 1A, 1.5=Stage 1B, 2=Stage 2A, etc).

As noted above, the weighting coefficients for each marker represent the weighted expression values relative to one other. In addition, the coefficients of Formula (II) are weighted relative to one another and relative to the MPI. As such, it will be readily appreciated by one of ordinary skill in the art that the coefficients of a formula for calculating CRM (e.g., Formula (II) above) can by multiplied by same factor as used for the MPI weighting coefficients. For example, in some embodiments, when the weighting coefficient for MAD2L1, GINS1, and KRT6A is 0.04; the weighting coefficient for SLC2A1 is 0.05; the weighting coefficient for FCGRT, TNIK, BCAM, and KDM6A is is −0.05; and the weighting coefficient for FAIM3 is −0.06; then CRM can be calculated according to the formula:

CRM=1.05*X+0.036Y+0.37Z Formula (III)

where X is the patient's calculated MPI; Y is the patient's age in years; and Z is a numerical description of the clinical score of the tumor stage (e.g, according to the TNM Classification of Malignant Tumors, which is a cancer staging system that describes the extent of a person's cancer and is known in the art, e.g., 1=stage 1A, 1.5=Stage 1B, 2=Stage 2A, etc).

In some cases, if an individual's calculated CRM is below the threshold, the individual has a decreased risk (e.g., increased likelihood of survival) relative to someone whose calculated CRM is above the threshold. In some cases, if an individual's calculated CRM is below the threshold, the individual can be said to have “low risk” (high likelihood of survival), and if an individual's calculated CRM is above the threshold, the individual can be said to have “high risk” (low likelihood of survival). In some cases (e.g., see working examples below) (e.g., when the weighting coefficient for MAD2L1, GINS1, and KRT6A is 0.04; the weighting coefficient for SLC2A1 is 0.05; the weighting coefficient for FCGRT, TNIK, BCAM, and KDM6A is is −0.05; and the weighting coefficient for FAIM3 is −0.06; and when CRM can be calculated according to Formula (III)), the threshold can be in a range of from 2 to 4 (e.g., from 2.1 to 3.9, from 2.2 to 3.8, from 2.3 to 3.7, from 2.5 to 3.5, from 2.7 to 3.3, from 2.8 to 3.2, from 2.8 to 3.1, from 2.85 to 2.99, from 2.0 to 2.2, from 2.2 to 2.4, from 2.4 to 2.6, from 2.6 to 2.8, from 2.8 to 3.0, from 3.0 to 3.2, from 3.2 to 3.4, from 3.4 to 3.6, from 3.6 to 3.8, from 3.8 to 4.0, from 2.1 to 2.3, from 2.3 to 2.5, from 2.5 to 2.7, from 2.7 to 2.9, from 2.9 to 3.1, from 3.1 to 3.3, from 3.3 to 3.5, from 3.5 to 3.7, from 3.7 to 3.9, 2.8, 2.85, 2.9, 2.92, 2.95, or 3.0). In some such cases, the threshold is 2.9. In some such cases, the threshold is 2.92. The threshold numbers can also be multiplied as discussed above by a factor relative to the rest of the terms in the equation. For example, in some cases (e.g., when the weighting coefficient for MAD2L1, GINS1, and KRT6A is 4; the weighting coefficient for SLC2A1 is 5; the weighting coefficient for FCGRT, TNIK, BCAM, and KDM6A is −5; and the weighting coefficient for FAIM3 is −6; and when CRM can be calculated according to Formula (II)), the threshold can be in a range of from 200 to 400 (e.g., from 210 to 390, from 220 to 380, from 230 to 370, from 250 to 350, from 270 to 330, from 280 to 320, from 280 to 310, from 285 to 290, from 200 to 220, from 220 to 240, from 240 to 260, from 260 to 280, from 280 to 300, from 300 to 320, from 320 to 340, from 340 to 360, from 360 to 380, from 380 to 400, from 210 to 230, from 230 to 250, from 250 to 270, from 270 to 290, from 290 to 310, from 310 to 330, from 330 to 350, from 350 to 370, from 370 to 390, 280, 285, 290, 292, 295, or 300). In some such cases, the threshold is 290. In some such cases, the threshold is 292.

In some cases, the clinical component of CRM can be defined as the sum of gender, stage, and age components from their coefficient in multivariate Cox regression (e.g., see Table 3 of working example 1). In some cases, CRM can be defined according to the formula:

CRM=0.022X+1.1Y Formula (IV)

where X is the patient's calculated MPI; and Y is the patient's clinical score (e.g., SEER score according to Table 3 of working example 1 below).

Providing a Prognosis.

By “providing a prognosis for an individual with NSCLC,” it is generally meant providing a determination of the severity of NSCLC (e.g., providing a statistical likelihood of survival), providing a classification of the subject's NSCLC into a subtype of the disease or disorder (e.g., high risk, intermediate risk, low risk, etc.), predicting the responsiveness of a patient to a therapy, predicting whether an aggressive therapy is called for (e.g., whether a more aggressive or less aggressive therapy is appropriate for the patient), and the like. Thus, providing an NSCLC prognosis can mean providing a prediction (e.g. a prediction of a subject's risk, e.g., risk of advancing to a more severe stage of NSCLC, risk of death, etc.; a prediction of likelihood of survival; a prediction of the course of progression, e.g. expected duration of the NSCLC, expectations as to whether the NSCLC will progress (e.g., to death), etc.; a prediction of a subject's responsiveness to treatment for the NSCLC, e.g., positive response, a negative response, no response at all; and the like).

In some embodiments, providing a prognosis is based on a comparison with a reference marker level representation. For example, in some embodiments, a marker level representation from an individual (representing the expression levels of two or more NSCLC markers in the individual) is compared to a marker level representation of a reference (e.g., a reference marker level representation or multiple reference marker level representations). In some cases, a reference marker level representation or multiple reference marker level representations is/are provided as part of a report.

In some embodiments, providing a prognosis is based on a comparison with multiple (i.e., two or more) reference marker level representations. The terms “reference” and “control” as used herein mean a standardized marker expression representation (reference marker level representation) to be used to interpret the analysis of a given patient and assign a prognostic, and/or responsiveness class thereto. The reference or control is typically an NSCLC expression profile or an NSCLC score that is obtained from a cell/tissue with a known diagnosis. In some cases (e.g., when comparing to multiple reference marker level representations), each marker level representations (e.g., each reference NSCLC score) is associated with a known prognosis or outcome (e.g, survival likelihood, a risk category, an appropriate treatment regimen, etc.). Thus, for example, a prognosis can be made by comparing the marker level representation of the individual with reference marker level representations that are associated with various outcomes.

For example, an NSCLC score of an individual (e.g, an MPI value, a CRM value, etc.) can be compared with two or more reference NSCLC scores where each reference NSCLC score is associated with a known outcome (e.g, survival likelihood, a risk category, an appropriate treatment regimen, etc.). In some cases, a prognosis can be made by comparing the marker level representation of the individual with reference marker level representations (or a single reference marker level representation) that are/is a known threshold. For example, an NSCLC score of an individual can be compared to reference NSCLC scores that are threshold values, where a score below (or in some cases equal to) the threshold is associated with a particular outcome (e.g., low risk, high survival likelihood, intermediate risk/survival likelihood, etc.) and/or a score above (or in some cases equal to) the threshold is associated with a particular outcome (e.g., high risk, low survival likelihood, intermediate risk/survival likelihood, etc.). In some cases, an NSCLC marker level representation may be compared to both a positive reference (e.g., reference NSCLC score, reference NSCLC profile, etc.) and a negative reference to obtain confirmed information regarding whether the individual has the prognosis of interest.

Comparing to a reference may comprise comparing the obtained NSCLC marker level representation to a NSCLC phenotype determination element to identify similarities or differences with the phenotype determination element, where the similarities or differences that are identified are then employed to provide the NSCLC assessment, e.g. prognose the NSCLC, monitor the NSCLC, determine a NSCLC treatment, etc. By a “phenotype determination element” it is meant an element, e.g. a tissue sample, a marker profile, a value (e.g. score), a range of values, and the like that is representative of a phenotype (in this instance, a NSCLC phenotype) and may be used to determine the phenotype of the subject, e.g. if the subject is healthy or is affected by NSCLC, if the subject has a NSCLC that is likely to progress, if the subject has a NSCLC that is responsive to therapy, etc.

For example, a NSCLC phenotype determination element may be a sample from an individual that has or does not have NSCLC, which may be used, for example, as a reference/control in the experimental determination of the marker level representation for a given subject. As another example, a NSCLC phenotype determination element may be a marker level representation, e.g. marker profile or score, which is representative of a NSCLC state and may be used as a reference/control (as described above) to interpret the marker level representation of a given subject. The phenotype determination element may be a positive reference/control, e.g., a sample or marker level representation thereof from a patient that has NSCLC, or that has NSCLC that is manageable by known treatments, etc. Phenotype determination elements are preferably the same type of sample or, if marker level representations, are obtained from the same type of sample as the sample that was employed to generate the marker level representation for the individual being monitored. For example, if a biopsy of an individual is being evaluated, the phenotype determination element would preferably be of a biopsy.

In certain embodiments, the obtained marker level representation is compared to a single phenotype determination element to obtain information regarding the individual being tested for NSCLC. In other embodiments, the obtained marker level representation is compared to two or more phenotype determination elements.

In certain embodiments, a similarity determination is made using a computer having a program stored thereon that is designed to receive input for a marker level representation obtained from a subject, to determine similarity to one or more reference profiles or reference scores, and return an NSCLC prognosis, e.g., to a user (e.g., lab technician, physician, NSCLC individual, etc.). Further descriptions of computer-implemented aspects of the invention are described below. In certain embodiments, a similarity determination may be based on a visual comparison of the marker level representation, e.g. NSCLC score, to a range of phenotype determination elements, e.g. a range of NSCLC scores, to determine the reference NSCLC score that is most similar to that of the subject. Depending on the type and nature of the phenotype determination element to which the obtained marker level profile is compared, the above comparison step yields a variety of different types of information regarding the biological sample that is assayed. As such, the above comparison step can yield a positive/negative prediction of the prognosis of NSCLC, a characterization of a NSCLC, information on the responsiveness of a NSCLC to treatment, and the like.

In some cases, a prognosis is a statistical likelihood of survival. Such statistical likelihoods can be obtained by comparing a marker level representation from an individual to marker level representations from a set of individuals with varying likelihoods of survival (e.g., an experimentally determined data set comprising survival data and NSCLC marker level representation data for individuals with NSCLC). Such comparisons can be used to correlate a range of marker level representations (e.g., a range of NSCLC scores) with a range of survival likelihoods. Thus, a marker level representation from an individual can be used to determine a statistical likelihood of survival for the individual.

Patients can be ascribed to high- or low-risk categories, or high-, intermediate- or low-risk categories, for overall survival, relapse-free survival, event-free survival, etc. depending on whether their marker level representation is higher or lower than the median (or another selected threshold) across a cohort of patients with the same disease. Thus, in some cases, a prognosis is a category of survival likelihood. In some cases, the category is high risk, intermediate risk, or low risk, wherein high risk is associated with a low likelihood of survival, low risk is associated with a high likelihood of survival, and medium risk is associated with an intermediate likelihood of survival.

The NSCLC marker level representation may be used to predict the course of disease progression and/or disease outcome, e.g. expected onset of the NSCLC, expected duration of the NSCLC, expectations as to whether the NSCLC will progress into a higher stage (e.g., whether a stage I NSCLC will progress to stage II), etc.

Alternatively or additionally, the NSCLC marker level representation may be employed to predict a subject's responsiveness to a treatment (e.g., a treatment regimen, a therapy, etc.) for the NSCLC, e.g., positive response, a negative response, no response at all, and the like. These predictive methods can be used to assist patients and physicians in making treatment decisions, e.g. in choosing the most appropriate treatment modalities for any particular patient. For example, the expression representation may be used to predict responsiveness to chemotherapy and to combinations of chemotherapy; to antibody therapy, to stem cell transplantation, etc. Additionally, the NSCLC marker level representation may be used on samples collected from patients in a clinical trial and the results of the test used in conjunction with patient outcomes in order to determine whether subgroups of patients are more or less likely to show a response to a new drug than the whole group or other subgroups. Further, such methods can be used to identify from clinical data the subsets of patients who can benefit from therapy. Additionally, a patient is more likely to be included in a clinical trial if the results of the test indicate a higher likelihood that the patient will have a poor clinical outcome if treated with more standardized treatments, and a patient is less likely to be included in a clinical trial if the results of the test indicate a lower likelihood that the patient will have a poor clinical outcome if treated with more standardized treatments.

As another example, the NSCLC marker level representation may be employed to monitor a NSCLC. By “monitoring” a NSCLC, it is generally meant monitoring a subject's condition, e.g. to inform an NSCLC prognosis, to provide information as to the effect or efficacy of an NSCLC treatment, and the like.

Treatment.

Subject methods and/or reports may include: recommending a treatment regimen based on the prognosis; prescribing a treatment regimen; and/or administering a treatment. In some embodiments, a “treatment recommendation” is provided for the individual based on a prognosis (e.g., guidance to a clinician as to a treatment recommendation for the individual based on the prognosis). Various treatments for individuals having NSCLC will be known to one of ordinary skill in the art as will treatment regimens associated with various prognoses.

Current treatments known in the art for patients with NSCLC include: surgery (e.g., wedge resection, lobectomy, pneumonectomy, sleeve resection, and the like); radiation therapy; chemotherapy (e.g., regional and/or systemic); targeted therapy (e.g., antibodies, inhibitors, and the like); laser therapy; cryotherapy; electrocautery; and the like. It is envisioned herein that more aggressive treatment regimens will be recommended, prescribed, and/or administered to those patients with lower likelihoods of survival (e.g., high risk category, low statistical likelihood of survival, and the like) while less aggressive treatment regimens will be recommended, prescribed, and/or administered to those patients with higher likelihoods of survival (e.g., low risk category, high statistical likelihood of survival, and the like). Which treatments are considered to be more or less aggressive will be known to one of ordinary skill in the art and it will be understood that decisions regarding how aggressive of a therapy to associate with which level of risk (likelihood of survival), may vary from patient to patient based on a variety of factors that can readily be discerned by one of ordinary skill in the art.

The terms “treatment”, “treating”, “treat” and the like are used herein to generally refer to obtaining a desired pharmacologic and/or physiologic effect. The effect can completely or partially prevent progression of a disease or symptom(s) (e.g., NSCLC) thereof and/or may be therapeutic in terms of a partial or complete cure for (e.g., reversal of) a disease and/or adverse effect attributable to the disease. The term “treatment” encompasses any treatment of a disease in a mammal, particularly a human, and includes: (a) inhibiting a disease and/or symptom(s), i.e., arresting development (e.g., preventing progression) of a disease and/or the associated symptoms; or (b) relieving the disease and the associated symptom(s), i.e., causing regression of the disease and/or symptom(s). In general, those in need of treatment can include those already having NSCLC.

Reports.

In some embodiments, a report is generated. A “report,” as described herein, is an electronic or tangible document which includes report elements that provide information of interest relating to the assessment of a subject and its results. In some embodiments, a subject report includes at least an NSCLC marker level representation (e.g., an NSCLC marker expression profile or an NSCLC score, e.g, an MPI value and/or a CRM value) as discussed in greater detail above. In some embodiments, a subject report includes an artisan's NSCLC assessment, e.g. NSCLC diagnosis, NSCLC prognosis, an analysis of a NSCLC monitoring, a treatment recommendation, a prescription, etc. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a protein level determination; 6) other features.

In some embodiments, the NSCLC assessment of the present disclosure is provided by providing (e.g., generating) a written report that can include at least one of: the NSCLC marker level representation (e.g., an NSCLC score) of the individual; a reference marker level representation (or multiple reference marker level representations); a prognosis (e.g., based on a comparison of the NSCLC marker level representation of the individual with a reference marker level representation); a recommended treatment regimen; a prescription for a treatment regimen; and the like. Thus, the subject methods may include a step of generating or outputting a report, which report can be provided in the form of an electronic medium (e.g., an electronic display on a computer monitor), or in the form of a tangible medium (e.g., a report printed on paper or other tangible medium). Any form of report may be provided.

A report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measuring the marker concentration in NSCLC patients versus healthy individuals, i.e. individuals that do not have and/or do not develop NSCLC. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, NSCLC stage, family medical history, and any other patient characteristics), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. Tumor, blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu). The report may include a results section. For example, the report may include a section reporting the results of a marker expression level determination assay, or a calculated NSCLC score.

The report may include an assessment report section, which may include information generated after processing of the data as described herein. The interpretive report can include a prognosis of NSCLC (e.g., a prediction of the survival likelihood of the patient). The interpretive report can include a characterization of NSCLC. The assessment portion of the report can optionally also include a recommendation(s). For example, where the results indicate that progression of NSCLC is likely, the recommendation can include a recommendation that therapy be altered (e.g., aggressive therapy be attempted), as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least an NSCLC marker level representation of the individual.

### Reagents, Systems and Kits

Also provided are reagents, systems and kits thereof for practicing one or more of the above-described methods. The subject reagents, systems and kits thereof may vary greatly. Reagents of interest include reagents specifically designed for use in producing the above-described marker level representations of NSCLC markers from a sample, for example, one or more detection elements (e.g. oligonucleotides for the detection of nucleic acids, e.g., primers, probes, etc.; antibodies or peptides for the detection of protein; and the like). In some instances, the detection element comprises a reagent to detect the expression of a single NSCLC marker, for example, the detection element may be a dipstick, a plate, an array, or cocktail that comprises one or more detection elements, e.g. one or more oligonucleotides, one or more sets of PCR primers, one or more antibodies, etc. which may be used to detect the expression of one or more NSCLC markers (in some cases, simultaneously).

One type of reagent that is specifically tailored for generating marker level representations, e.g. NSCLC marker level representations, is a collection of antibodies that bind specifically to the protein markers, e.g. in an ELISA format, in an xMAP™ microsphere format, on a proteomic array, in suspension for analysis by flow cytometry, by western blotting, by dot blotting, or by immunohistochemistry. Methods for using the same are well understood in the art. These antibodies can be provided in solution. Alternatively, they may be provided pre-bound to a solid matrix, for example, the wells of a multi-well dish or the surfaces of xMAP microspheres.

Another type of such reagent is an array of probe nucleic acids in which the genes of interest are represented. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Another type of reagent that is specifically tailored for generating marker level representations of genes, e.g. NSCLC genes, is a collection of gene specific primers that is designed to selectively amplify such genes (e.g., using a PCR-based technique, e.g., real-time RT-PCR). Gene specific primers and methods for using the same are described in U.S. Pat. No. 5,994,076, the disclosure of which is herein incorporated by reference.

Of interest are arrays of probes, collections of primers, or collections of antibodies that include probes, primers or antibodies (also called reagents) that are specific for 2 or more gene/proteins selected from the group consisting of MAD2L1, GINS1, SLC2A1, KRT6A, FCGRT, TNIK, BCAM, KDM6A, and FAIM3; and in some instances for a plurality of these genes/polypeptides, e.g., 3 or more, 4 or more, 5 or more, 6 or more, 7 or more, 8 or more, or all 9. Of interest are arrays of probes, collections of primers, or collections of antibodies that include probes, primers or antibodies (also called reagents) that are specific for 2 or more gene/proteins selected from the group consisting of MAD2L1, GINS1, SLC2A1, KRT6A, FCGRT, TNIK, BCAM, KDM6A, FAIM3, and FSCN1; and in some instances for a plurality of these genes/polypeptides, e.g., 3 or more, 4 or more, 5 or more, 6 or more, 7 or more, 8 or more, 9 or more, or all 10. The subject probe, primer, or antibody collections or reagents may include reagents that are specific only for the genes/proteins that are listed above, or they may include reagents specific for additional genes/proteins that are not listed above, such as probes, primers, or antibodies specific for genes/proteins whose expression pattern are associated with NSCLC.

As noted above, detection reagents can be provided as part of a kit. Thus, the disclosure further provides kits for measuring two or more NSCLC markers selected from: MAD2L1, GINS1, SLC2A1, KRT6A, FCGRT, TNIK, BCAM, KDM6A, and FAIM3 in a biological sample. The disclosure also provides kits for measuring two or more NSCLC markers selected from: MAD2L1, GINS1, SLC2A1, KRT6A, FCGRT, TNIK, BCAM, KDM6A, FAIM3, and FSCN1 in a biological sample.

Procedures using these kits can be performed by clinical laboratories, experimental laboratories, medical practitioners, or private individuals. The kits of the invention may comprise amplification and/or sequencing primers, and/or hybridization primers or antibodies for protein determination. The kit may optionally provide additional components that are useful in the procedure, including, but not limited to, buffers, developing reagents, labels, reacting surfaces, means for detection, control samples, standards, instructions, and interpretive information.

Kits of the subject disclosure may include the above-described arrays, gene-specific primers (e.g., primer collections), or protein-specific antibody collections. Kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject kits may also include one or more NSCLC phenotype determination elements, which element is, in many embodiments, a reference or control sample or marker representation that can be employed, e.g., by a suitable experimental or computing means, to make a NSCLC prognosis based on an “input” marker level profile, e.g., that has been determined with the above described marker determination element. Representative NSCLC phenotype determination elements include samples from an individual known to have high- or low-risk NSCLC, databases of marker level representations, e.g., reference or control profiles or scores, and the like, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that may be present is a website address which may be used via the internet to access the information at a removed site. Any convenient means may be present in the kits.

In addition to instructions for using the components of the kit, the kit can further include instructions of analyzing the data acquired from the assays described herein. For example, the instructions can include a graph and/or table of known statistics for the probabilities of overall survival and progression free survival following anthracycline-based chemotherapy or anthracycline-based chemotherapy in conjunction with anti-CD20 immunotherapy for persons with lymphomas having differing expression of the genes of the invention. In addition, instructions can be provided to interpret these graphs and/or tables. These graphs and/or tables and instructions would be generally recorded on a suitable recording medium, for example, printed on a substrate such as paper or plastic. Alternatively, these graphs and/or tables and instructions can be provided on an electronic storage data file present on a suitable computer readable storage medium, e.g. CD-ROM, diskette, etc. In some embodiments, the actual graphs and/or table and instructions are not present in the kit, but means for obtaining the graphs/tables and instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Computer-Implemented Methods, Systems and Devices.

The methods of the present disclosure can be computer-implemented, such that method steps (e.g., assaying (e.g., measuring), calculating, comparing, and the like) are be automated in whole or in part. Accordingly, the present disclosure provides methods, computer systems, devices and the like in connection with computer-implemented methods of determining a likelihood of survival in a subject with NSCLC (e.g., statistical likelihood, categorical likelihood, etc., as described above).

For example, the method steps, including measuring expression levels of NSCLC markers, calculating an NSCLC marker level representation, comparing an NSCLC marker level representation to a reference marker level representation, generating a report, and the like, can be completely or partially performed by a computer program product. Values obtained can be stored electronically, e.g., in a database, and can be subjected to an algorithm executed by a programmed computer.

For example, the methods of the present disclosure can involve inputting the expression levels (e.g. raw values, normalized values, weighted values, and/or normalized and weighted values) of 2 or more NSCLC markers (described above) into a computer programmed to execute an algorithm to perform the comparing step described herein, and generate a report as described herein, e.g., by displaying or printing a report to an output device at a location local or remote to the computer.

The present invention thus provides a computer program product including a computer readable storage medium (e.g., a nontransitory computer-readable storage medium) having a computer program stored on it. The program can, when read by a computer, execute relevant calculations based on values obtained from analysis of one or more biological samples from an individual. The computer program product has stored therein a computer program for performing the calculation(s).

The present disclosure provides systems for executing the program described above, which system generally includes: (i) a central computing environment; (ii) an input device, operatively connected to the computing environment, to receive patient data (e.g., NSCLC marker expression level data, clinical data from the patient, etc. as described above); (iii) an output device, connected to the computing environment, to provide information to a user (e.g., medical personnel, clinician, and the like); and (iv) an algorithm executed by the central computing environment (e.g., a processor), where the algorithm is executed based on the data received by the input device, and where the algorithm can in some cases calculate a value and/or category, which value and/or category is indicative of the likelihood of survival.

### Computer Systems

The present disclosure also provides computer systems for calculating an NSCLC marker level representation for an individual, and/or for providing a prognosis of for an individual. The computer systems include a processor and memory operably coupled to the processor, where the memory programs the processor to perform at least one of the following tasks: receive assay data (e.g., expression levels of two or more NSCLC markers) from a biological sample from a subject; calculate an NSCLC marker level representation based on expression levels; compare a calculated NSCLC marker level representation to a reference marker level representation; and provide a prognosis for the individual (e.g., which may include calculating a likelihood of survival for the individual), based on results of comparing a calculated NSCLC marker level representation to a reference marker level representation. In certain aspects, when calculating a likelihood of survival in the subject, the system integrates one or more additional clinical factors (e.g., calculates a composite risk model (CRM), e.g., where the CRM is the NSCLC marker level representation).

Computer systems may include a processing system, which generally comprises at least one processor or processing unit or plurality of processors, memory, at least one input device and at least one output device, coupled together via a bus or group of buses. In certain embodiments, an input device and output device can be the same device. The memory can be any form of memory device, for example, volatile or non-volatile memory, solid state storage devices, magnetic devices, etc. The processor can comprise more than one distinct processing device, for example to handle different functions within the processing system.

An input device receives input data and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data can come from different sources, for example keyboard instructions in conjunction with data received via a network.

Output devices produce or generate output data and can comprise, for example, a display device or monitor in which case output data is visual, a printer in which case output data is printed, a port for example a USB port, a peripheral component adaptor, a data transmitter or antenna such as a modem or wireless network adaptor, etc. Output data can be distinct and derived from different output devices, for example a visual display on a monitor in conjunction with data transmitted to a network. A user can view data output, or an interpretation of the data output, on, for example, a monitor or using a printer. The storage device can be any form of data or information storage means, for example, volatile or non-volatile memory, solid state storage devices, magnetic devices, etc.

In use, the processing system may be adapted to allow data or information to be stored in and/or retrieved from, via wired or wireless communication means, at least one database. The interface may allow wired and/or wireless communication between the processing unit and peripheral components that may serve a specialized purpose. In general, the processor can receive instructions as input data via input device and can display processed results or other output to a user by utilizing output device. More than one input device and/or output device can be provided. A processing system may be any suitable form of terminal, server, specialized hardware, or the like.

A processing system may be a part of a networked communications system. A processing system can connect to a network, for example the Internet or a WAN. Input data and output data can be communicated to other devices via the network. The transfer of information and/or data over the network can be achieved using wired communications means or wireless communications means. A server can facilitate the transfer of data between the network and one or more databases. A server and one or more databases provide an example of an information source.

Thus, a processing computing system environment may operate in a networked environment using logical connections to one or more remote computers. The remote computer may be a personal computer, a server, a router, a network PC, a peer device, or other common network node, and typically includes many or all of the elements described above.

Certain embodiments may be described with reference to acts and symbolic representations of operations that are performed by one or more computing devices. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of well-known computing systems, environments, and configurations that may be suitable for use with an embodiment include, but are not limited to, personal computers, handheld or laptop devices, personal digital assistants, multiprocessor systems, microprocessor-based systems, programmable consumer electronics, network, minicomputers, server computers, web server computers, mainframe computers, and distributed computing environments that include any of the above systems or devices.

Embodiments may be described in a general context of computer-executable instructions, such as program modules, being executed by a computer. Generally, program modules include routines, programs, objects, components, data structures, etc., that perform particular tasks or implement particular abstract data types. An embodiment may also be practiced in a distributed computing environment where tasks are performed by remote processing devices that are linked through a communications network. In a distributed computing environment, program modules may be located in both local and remote computer storage media including memory storage devices.

### Computer Program Products

The present disclosure provides computer program products that, when executed on a programmable computer such as that described above, can carry out the methods of the present disclosure. As discussed above, the subject matter described herein may be embodied in systems, apparatus, methods, and/or articles depending on the desired configuration. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g. video camera, microphone, joystick, keyboard, and/or mouse), and at least one output device (e.g. display monitor, printer, etc.).

Computer programs (also known as programs, software, software applications, applications, components, or code) include instructions for a programmable processor, and may be implemented in a high-level procedural and/or object-oriented programming language, and/or in assembly/machine language. As used herein, the term “machine-readable medium” refers to any nontransitory computer program product, apparatus and/or device (e.g., magnetic discs, optical disks, memory, etc.) used to provide machine instructions and/or data to a programmable processor, including a machine-readable medium that receives machine instructions as a machine-readable signal.

It will be apparent from this description that aspects of the present invention may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

In addition, the processor(s) may be, or may include, one or more programmable general-purpose or special-purpose microprocessors, digital signal processors (DSPs), programmable controllers, application specific integrated circuits (ASICs), programmable logic devices (PLDs), trusted platform modules (TPMs), or the like, or a combination of such devices. In alternative embodiments, special-purpose hardware such as logic circuits or other hardwired circuitry may be used in combination with software instructions to implement the techniques described herein.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pi); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

Gene expression datasets were pooled to derive a robust and internally validated molecular prognostic index (MPI) for stage I non-squamous NSCLC that reflects multiple distinct aspects of tumor heterogeneity, including the tumor microenvironment. To facilitate its clinical implementation, a quantitative real time polymerase chain reaction (qRT-PCR) assay was developed that is readily applicable to routinely obtained diagnostic tumor samples that are formalin-fixed and paraffin-embedded (FFPE) tumor samples. The MPI was also integrated with clinical and pathological variables to determine a composite risk model (CRM), thus enabling individualized risk prediction.

The expression of thousands of genes are correlated with outcome in NSCLC and many different subsets of these carry prognostic power. However, this phenomenon also increases the chance of overfitting in the training set, which along with the other design shortcomings has likely contributed to the lack of published validation studies for the majority of signatures. From a technical standpoint, a common pitfall with gene expression-based prognostic tools in general is over-training to a specific cohort with subsequent failure to perform well in new patient samples. This can persist even when internal cross-validation of training sets are used, since a single round of this procedure is strongly affected by the initial split. Multiple rounds of internal cross-validation were therefore applied to a balanced training set to identify a consensus set of gene features for incorporation into a final 9-gene model. In addition, we empirically demonstrate below that successful internal cross-validation becomes increasingly likely if the dataset is sufficiently large, which supports the approach of consolidating data from multiple published sources. The combination of the biologically-driven approach and these statistical features facilitated the establishment of a robust predictor that could be validated using a different technique (qPCR) and on FFPE tissues.

### Patients and Methods

Lung Cancer Gene Expression Datasets.

We identified 7 published datasets (Table 4) comprising gene expression profiles of NSCLC patients for which overall (OS) or disease-specific (DSS) survival data were annotated, and which included at least 40 stage I non-squamous NSCLC patients (see below). Gene expression data were obtained as described in Table 4. Measurements from different platforms were combined into one meta-cohort (n=1106) for analysis using the correlation structure of the underlying microarray data for normalization and standardization (supplementary methods), and split into balanced training and validation sets. Patient characteristics and clinical annotations were individually reviewed and standardized using stage assignments per AJCC version 6 (2002), corresponding to the era for which the majority of datasets were assembled. Patients who were alive beyond 5 years were censored at 5 years. Characteristics of the microarray training, microarray validation, and qPCR FFPE validation sets are summarized in Table 1.

TABLE 1
Cohorts of NSCLC patients included for training,
testing, and validation of the MPI.

| | Microarray | Microarray | qPCR |
|---|---|---|---|
| | Training | Test | Validation |
| | set | set | set |
| Number of samples | 563 | 543 | 98 |
| Median age (years) | 63 | 62 | 71 |
| Gender | | | |
| Male | 261 | 259 | 34 |
| Female | 267 | 244 | 64 |
| Stage | | | |
| IA | 169 | 175 | 63 |
| IB | 188 | 166 | 23 |
| IIA | 21 | 15 | 7 |
| IIB | 51 | 61 | 5 |
| IIIA | 43 | 40 | 0 |
| IIIB | 9 | 10 | 0 |
| IV | 5 | 5 | 0 |
| Smoking status | | | |
| Current smoker | 35 | 33 | — |
| Ever smoker | 52 | 59 | — |
| Former smoker | 135 | 141 | — |
| Never smoker | 82 | 83 | — |
| Median followup (months) | 49 | 51 | 44 |
| Number of deaths | 191 | 182 | 33 |

### Statistical Analyses of Microarray Datasets

Normalization, Integration, and Annotation of Expression Datasets.

Affymetrix data, raw CEL files were downloaded and normalized with the MAS5 algorithm using Custom chip Definition Files that map to Entrez gene identifiers (Brainarray v. 17 “brainarray.” followed by “mbni.med.” followed by “umich.” Followed by “edu/Brainarray/Database” followed by “/CustomCDF/CDF_” followed by “download.asp”), followed by log 2 transformation and quantile normalization. For each gene i in each Affymetrix dataset, a gene correlation neighborhood was defined as those other genes whose correlation with gene i was at least rₘᵢₙ. For non-Affymetrix data such as long oligonucleotide Agilent or Operon arrays with multiple probes corresponding to a particular gene, the single probe was selected that was most correlated with the members of its gene correlation neighborhood defined above. Within each dataset, every gene was standardized so that its mean expression was zero, and standard deviation was 1 across the stage I samples. To this end, only the 7 cohorts that had at least 40 stage I samples were included (Table 4). The expression matrices from each study could then be merged into one meta-cohort of 1106 samples (Table 4).

TABLE 4
Publicly available NSCLC gene expression datasets used in this study.

| Accession | ca00182 | GSE31210 | ca00191 | Roepman LungCancer | GSE13213 | ca00153 | GSE5843 |
|---|---|---|---|---|---|---|---|
| 1^{st} author | Shedden | Okayama | Bhattacharjee | Roepman | Tomida | Beer | Larsen |
| PMID | 18641660 | 22080568 | 11707567 | 19118056 | 19414676 | 12118244 | 17504995 |
| Subtypes | Adeno. | Adeno | Adeno. | Adeno, SCC | Adeno. | Adeno. | Adeno. |
| | | stage 1, 2 | | | | | |
| Platform | Affymetrix | Affymetrix | Affymetrix | Agilent 44K whole | Agilent 44K | Affymetrix | PC Human |
| | U133A | U133 | HgU95Av2 | genome | whole genome | Hu6800 | Operon v2 |
| | | Plus 2.0 | | | | | 21k) |
| n | 443 | 246 | 186 | 172 | 117 | 96 | 48 |
| Treated | no | no | no | no | no | no | no |
| pre-biopsy | | | | | | | |
| Survival | OS, | OS, | OS | OS, RFS | OS, relapse | OS | OS, DSS, |
| data | PRFS | relapse | | | | | RFS |
| Journal | Nat. | Cancer | PNAS | Clin Cancer Res. | J Clin Oncol | Nat Med. | Clin Cancer |
| | Med. | Res. | (2001) | 2009 Jan. | 2009 Jun. | 2002 | Res. 2007 |
| | 2008 | 2011 Nov. | 98: 13790-95 | 1; 15(1): 284-90 | 10; 27(17): 2793-9 | Aug; 8(8): 816-24. | May |
| | 14: 822-7 | 11 | | | | | 15; 13(10): 2946-54 |

The meta-cohort was split into nearly equal sized training and test sets that were balanced for original cohort membership, event vs. non-event, and time to event. Briefly, for each of the 7 analyzed cohorts, patients were separated by status (alive/dead) at last follow-up. Within each category, patients were then sorted by follow-up time. This sorted list was stepped through, randomly assigning patients to the training or test cohort. The probability of assignment to training/test cohorts was set to be 0.5, giving approximately equally sized sets to ensure robustness. Stage was coded as IA=0, IA/IB=0.5, IB=1, IIA=2, IIA/B=2.5, IIB=3 etc. OS and DSS times were converted to months, with Death=1 and Censored=0. Patients alive at 5 years were censored at that time to alleviate issues related to comorbidities.

Genes with >50% missing expression values in either the training or validation set were excluded. Cox proportional hazards regression was performed on each gene in the training set separately across all patients, only stage I patients, and only non-stage I patients. Missing values were excluded from univariate analyses. Significant genes (P<0.01, log-likelihood test) were selected for further robustness testing using an ensemble approach. Specifically, Cox regression was performed for the genes passing the initially filtering using half the training set, randomly selected. This procedure was repeated 1000 times, and then for each gene the average z-score was calculated for association with survival, as well as the number of significant trials (P<0.01 by log-likelihood test).

Missing values were imputed on the training and validation sets separately by the k-Nearest-Neighbors algorithm (k=15) for clustering and GLMnet analyses described below. Gene expression data for the prognostic genes in the training set were clustered using AutoSOME with thresholds of P<0.05 for sample and gene cluster membership, 100 ensemble runs, and Pearson correlation as the distance metric. Gene cluster memberships defined by AutoSOME were assessed for enrichment of gene sets by hypergeometric test with an empirical FDR correction for multiple hypothesis testing.

The 5 most prognostic genes (ranked by the frequency with which they achieved P<0.01 in robustness testing) from each of the 4 largest clusters were selected as candidate genes for the final model building. Multivariate Cox models were built from these 20 genes using GLMnet with a Lasso penalty for feature selection, and 10-fold internal cross-validation. Genes were ranked according to how many times they were included in a multivariate model after 100 iterations of repeated 10-fold cross-validation within GLMnet. We performed this bootstrapping because the way in which patients are randomly assigned to the cross-validation groups can spuriously affect gene selection. Hence individual cross-validation runs do not select exactly the same sets of prognostic genes. A final prognostic index was defined by fitting a Cox model to genes that were incorporated in >90% of cross-validation runs. Model coefficients were rounded to the nearest integer.

Cell Sorting and Whole Transcriptome Sequencing.

Fresh human lung tumor samples were dissociated into single cell suspensions for flow cytometry analysis and cell sorting. Total RNA extracted from sorted cell populations was reverse transcribed, amplified, and used to construct DNA libraries for sequencing. This research was approved by the Institutional Review Board of Stanford University School of Medicine. All participants provided written informed consent. Additional details are provided in the Supplemental Methods.

Gene Expression Analysis by qPCR.

RNA was purified from lung tumor tissue obtained from FFPE blocks. Synthesis of cDNA was performed from 1 μg of total RNA using the High Capacity cDNA Reverse Transcription Kit (Applied Biosystems), and qPCR was performed using TaqMan Gene Expression Assays (Applied Biosystems). Relative gene expression was determined by the ΔΔCt method.

Composite Model for Risk Stratification.

A prognostic model based on age, gender, and stage (relative to stage IA) was fit to SEER data. The prognostic value of the clinical score was tested in multivariate Cox regression with the MPI. A composite risk model (CRM) score was defined as the combination of these indicators weighted by their coefficient in the multivariate Cox model. The relative prognostic value of the clinical, molecular, and composite models were compared using the area under the receiver-operator curve (AUROC), Net Reclassification Improvement (NRI), and Integrated Discrimination Improvement (IDI), assessed for the utility in assigning risk of death at 5 years.

Assessment of Required Sample Sizes.

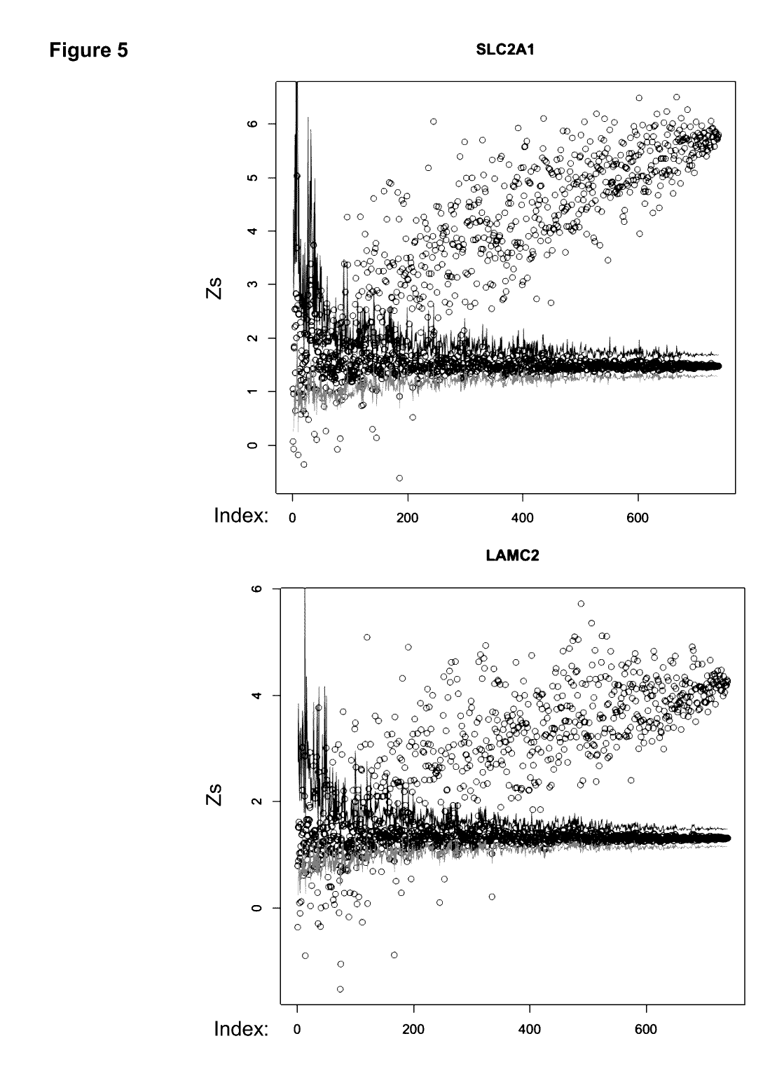

We randomly sub-sampled n cases from our meta-cohort and evaluated the prognostic power of SLC2A1 and LAMC2 as n was increased. The z-score for association between expression of these genes and survival increased as a function of n as expected (FIG. 5).

TMA Cohort and Construction.

Patient samples were retrieved from the surgical pathology archives at the Stanford Department of Pathology and linked to a clinical database using the Cancer Center Database and STRIDE Database tools from Stanford. We reviewed patients who had surgically treated disease and paraffin embedded samples from 1995 through June, 2010 for inclusion. Medical charts were reviewed to clinically annotate the tumor specimens with demographic, operative procedures, imaging data, and follow-up. Pathology reports were reviewed to confirm specimen type, site, pathology, stage, histology, invasion status and operative procedure.

Recurrence was defined by imaging or biopsy and patients with advanced disease or who did not have at least 6 months of follow-up were censored for further analyses. The National Death Index (NDI) was used to define vital status through Oct. 30, 2010. Patients not recorded as deceased in NDI were assumed to be alive except for those who had left the country or were from other countries (who were censored) since the NDI relies on a social security number for vital status assessment. Synchronous tumors resected over time were eligible for prognostic assessment in patients with two primaries. All aspects of this study were IRB approved prior to its initiation in accordance with the Declaration of Helsinki guidelines for the ethical conduct of research.

The Stanford Lung Cancer TMA was developed from surgical specimens that contained viable tumor from duplicate slides that were reviewed by a board-certified pathologist (RBW). The area of highest tumor content was marked for coring blocks corresponding to the slides. We used 2 mm cores to build the tissue microarray. These cores were aligned by histology and stage and negative controls included a variety of benign and malignant tissues (65 cores) that included normal non-lung tissue (12 cores), abnormal non-lung tissue (13 cores), placental markers (23 cores) and normal lung (17 cores). Normal lung consisted of a specimen adjacent, but distinct, from tumor over the years 1995 through 2010 to assess the variability of staining by year. OligoDT analysis was performed on the finished array to assess the architecture of selected cores and adequacy of tissue content prior to target immunohistochemistry (IHC) analysis. A co-registered hematoxylin- and eosin-stained slide was used as well to verify tumor location for cases where this was unclear on initial inspection.

Immunohistochemistry.

Serial 4 μm sections were cut from FFPE specimens and processed for IHC using the Ventana BenchMark XT automated immunostaining platform (Ventana Medical Systems/Roche, Tucson, Ariz.). Rabbit polyclonal anti-GLUT-1 antibody was obtained from Millipore (cat. #07-1401). The level of GLUT-1 immunostaining was determined by the consensus from three independent physician observers; each observer was blinded to the results of qPCR for SLC2A1.

RNA Isolation from FFPE Lung Tumor Tissue.

Lung tumor tissue was isolated from FFPE blocks with a 14-gauge needle after examining a hematoxylin- and eosin-stained section for tumor purity. RNA was purified from 3-6 mg of FFPE tissue using the Allprep DNA/RNA FFPE Kit (Qiagen) with the following modifications. Minced FFPE tissue was washed in HistoClear for 3 min at 50° C., pelleted, and then washed in 100% ethanol at room temperature. The tissue pellet was allowed to dry for 10 min before proceeding to proteinase K treatment. The proteinase K incubation was performed at 65° C. for 15 min. The heat-treatment step for reversal of formaldehyde modifications of the nucleic acid was performed at 70° C. for 20 min. On-column DNase I treatment was performed according to the manufacturer's protocol. RNA was eluted in sterile RNase-free water and assessed for purity and concentration with a NanoDrop Spectrophotometer. Total RNA yield from each sample ranged between 1 and 16 μg (median, 5 μg).

Quantitative Real-Time PCR.

Synthesis of cDNA was performed from 1 μg of RNA using the High Capacity cDNA Reverse Transcription Kit (Applied Biosystems) in a 20 μL reaction volume. Next, 14 cycles of pre-amplification was performed on 80 ng cDNA using the TaqMan PreAmp Master Mix Kit (Applied Biosystems) according to the manufacturer's specifications with a pool of multiplexed TaqMan assays in 25 μL reaction volumes. The pre-amplified products were diluted 1:20 in water and used in 10 μL reaction volumes for qPCR with a final concentration of 1× TaqMan Gene Expression Master Mix (Applied Biosystems) and 1× TaqMan Gene Expression Assays (Applied Biosystems). Each TaqMan gene expression assay was performed in 384 well optical plates on an ABI Prism HT7900 Real Time PCR system (Applied Biosystems). Thermal cycling conditions were: 50° C. for 2 minutes; 95° C. for 10 minutes; 40 cycles of denaturation at 95° C. for 15 seconds and annealing and extension at 60° C. for 1 minute. Reactions were performed in triplicate, and the results were averaged.

Gene Expression Analysis from qPCR Data.

Relative gene expression was determined by the ΔΔCt method. For each qPCR reaction, the fractional cycle number at which the amount of amplified target reached a fixed threshold (Ct) was determined using SDS v2.4 software (Applied Biosystems). Levels of the 9 target genes were each normalized to the average expression levels of AGPAT1 and PRPF40A, endogenous housekeeping genes that were chosen due to (1) low variance of expression within the microarray datasets and (2) moderate level of expression on par with the 9 prognostic genes. For calibration, we used cDNA derived from Stratagene Universal Human Reference RNA. Clontech reference may alternatively be used.

Human Lung Tumor Dissociation and Flow Cytometry.

Fresh human lung tumor samples were obtained from Stanford Tissue Bank. Tissues were cut into small pieces and dissociated into single cell suspensions by 45 minutes of collagenase I (STEMCELL Technologies) digestion. Dissociated single cells were suspended at 10⁷ per mL in staining buffer (HBSS with 2% heat-inactivated calf serum). After blocking with 10 μg/mL rat IgG, cells were stained with the antibodies listed below. Stained cells were washed and re-suspended in staining buffer with 1 μg/mL DAPI, analyzed, and sorted with a FACS Aria II cell sorter (BD Biosciences). Antibodies used for experiments included CD45-A700 (pan-immune cell marker), CD31-PE (endothelial cell marker), EpCAM-APC (epithelial cell marker), CD10-PE-Cy7 (fibroblast marker). All antibodies were obtained from BioLegend.

Whole Transcriptome Sequencing and Analysis.

Total RNA was extracted from sorted cell populations (range: 500 to 25,000 cells). A median of ˜30 ng total RNA was amplified using the Ovation RNA-Seq System V2 (Nugen). 1 μg of resulting cDNA was sheared by sonication (Covaris S2 System) to an average size of 150-200 bp and used to construct DNA libraries for sequencing using NEBNext DNA Library Prep Master Mix (New England Biolabs). Finally, constructed DNA libraries were sequenced in the Stanford Stem Cell Institute Genome Center on an Illumina HiSeq 2000. Approximately 50 million fragments were sequenced using paired end, 100 bp reads from each sample. Reads were aligned to the hg19 human genome assembly using STAR, and Cufflinks were used to quantitate FPKM (fragments per kilobase of exon per million mapped sequence reads).

SEER Cohort and Analysis.

The Surveillance, Epidemiology, and End Results (SEER) program collects and stores data of cancer patients from certain geographical areas in the USA. We used the SEER database to identify patients diagnosed with non-squamous NSCLC from 2004 to 2010. We excluded patients diagnosed at autopsy, patients who did not undergo surgery, and patients who died within 1 month of diagnosis. There were 28,691 cases identified in the SEER cohort.

SEER data were used to obtain sex, stage, grade, age, and whether surgical resection was performed. Staging was according to the AJCC 6^{th} edition. Grade was categorized as well-differentiated, moderately-differentiated, poorly-differentiated, undifferentiated, or unknown. Surgical resection included segmentectomy, wedge resection, lobectomy, partial pneumonectomy and total pneumonectomy.

Using SEER data, OS was defined as the time from NSCLC diagnosis to death. For patients surviving past Dec. 31, 2010, they were classified as censored. Cause of death was determined from SEER, which utilizes state death certificates as its primary source. Patients were censored at time of non-cancer related death for CSS. The method of Kaplan and Meier was used for survival estimates. Log-rank tests were used to compare sex, grade, and age groups. Univariate and multivariate proportional hazards models stratified by surgical resection status were used to assess the effect of sex, stage, grade, and age on OS and CSS. Significance was set at P≦0.05, and all tests were two-tailed. SAS version 9.3 (SAS Institute, Cary, N.C.) was used for all analysis.

### Results:

Identification of Prognostic Genes in NSCLC from Pooled Published Gene Expression Data.

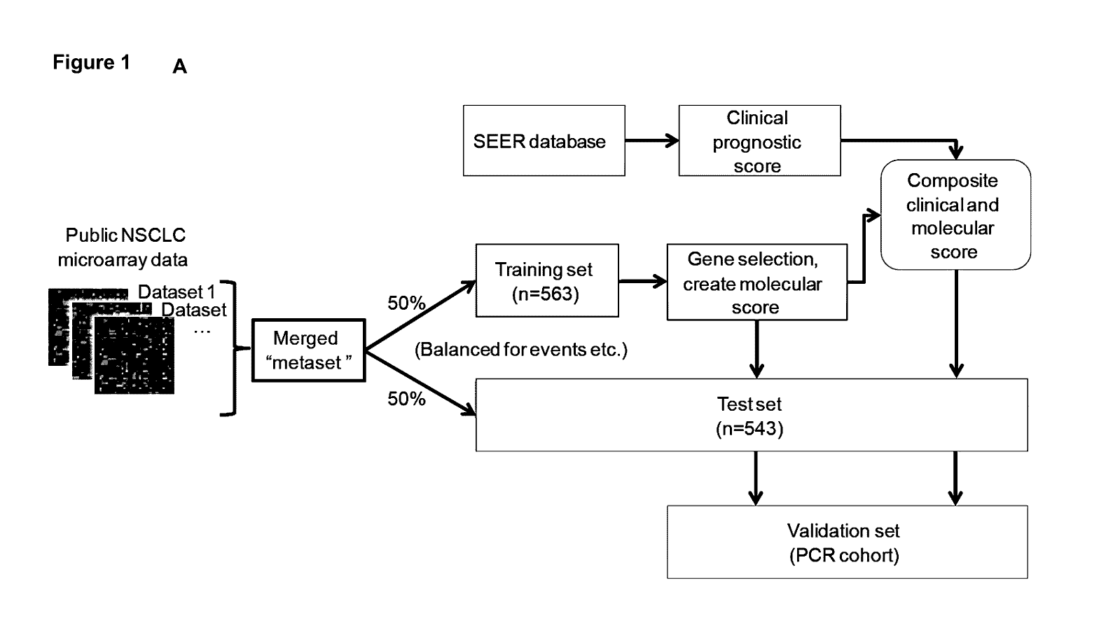

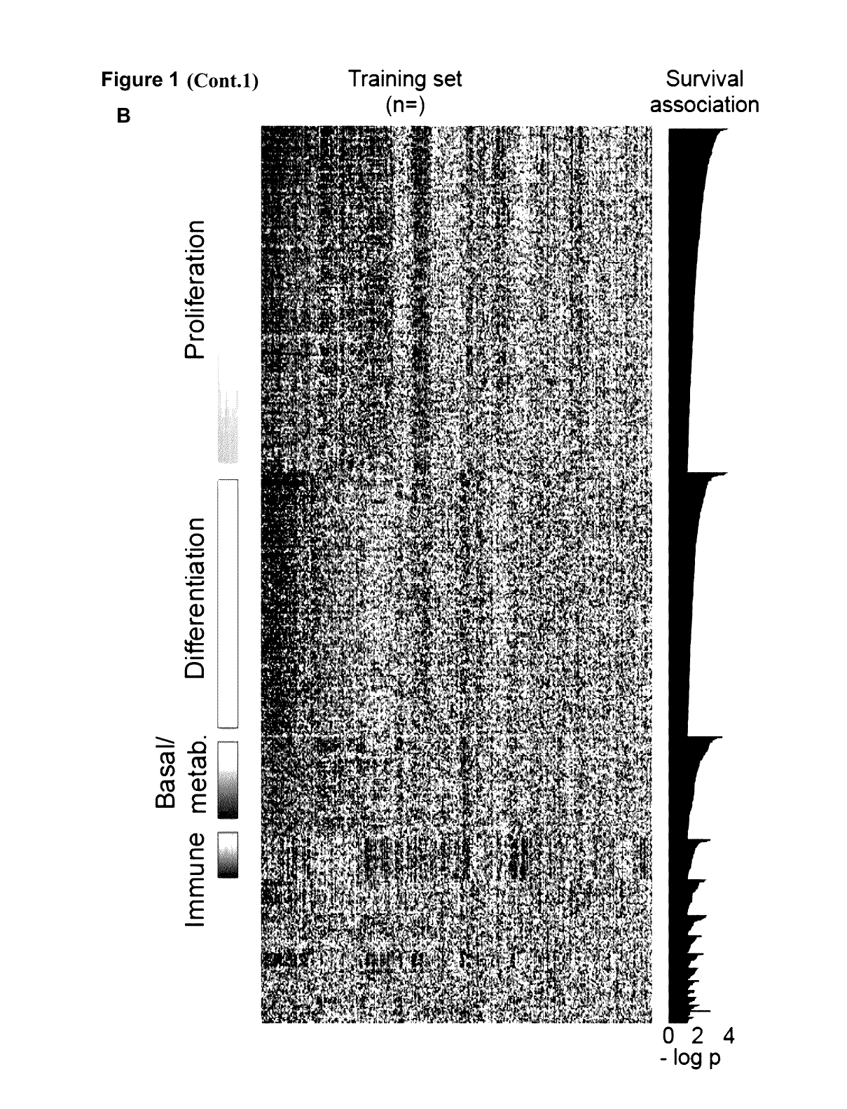

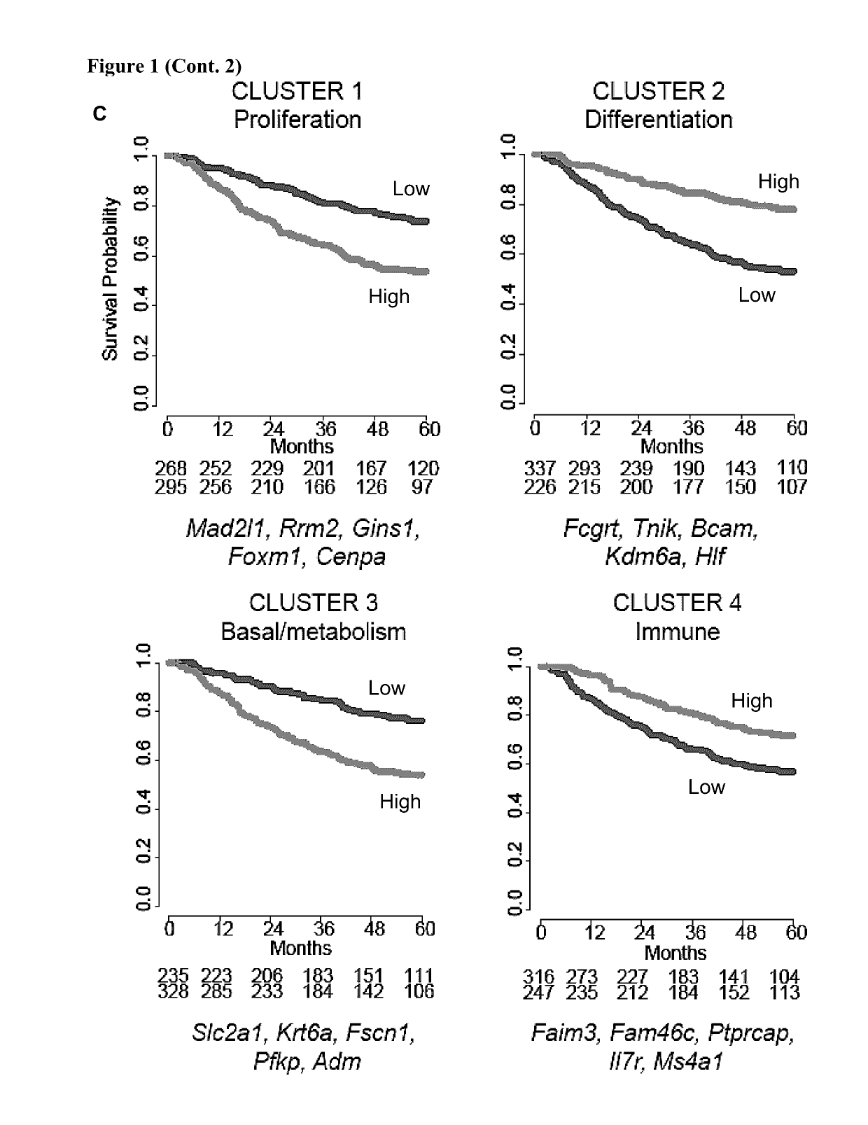

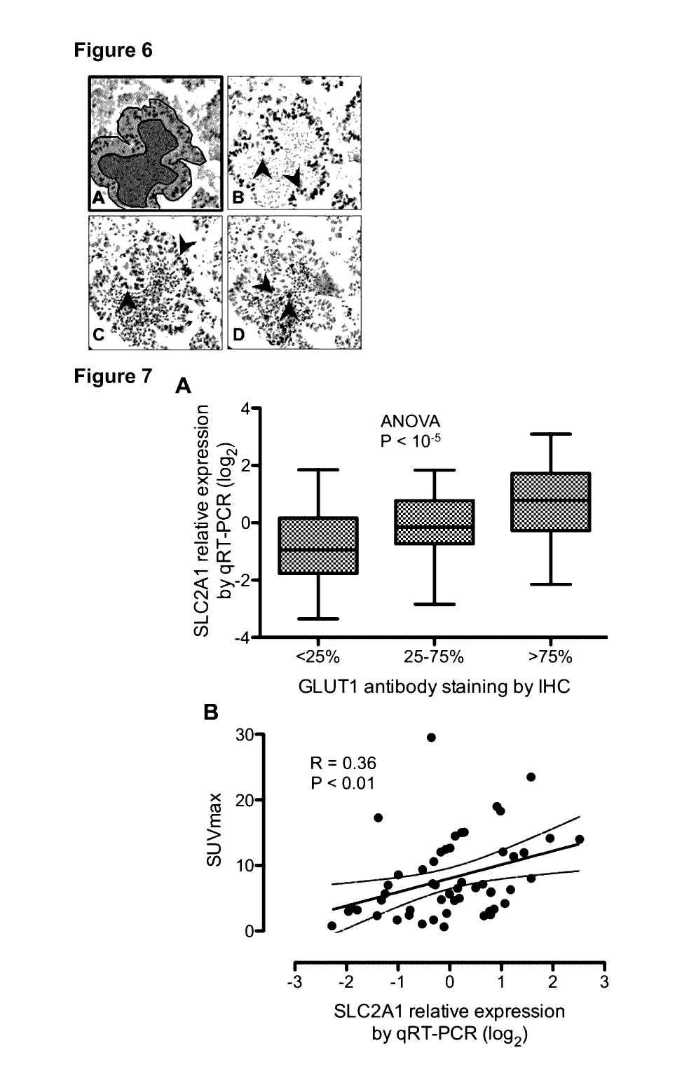

We assembled a compendium of non-squamous lung NSCLC datasets from the literature, and combined them into a single meta-cohort containing 1106 tumors for subsequent analyses. Several previously reported prognostic gene expression signatures in NSCLC have been difficult to validate in subsequent studies, partly due to small cohort sizes. We investigated the impact of training set sample size on prognostic power for two genes, SLC2A1 and LAMC2, both of which have been identified as prognostic in lung cancer in some but not all previous studies. A sample size of at least 350-400 patients was required for reliable assessment of prognostic power, even for an apparently strong association such as for SLC2A1 (FIG. 5). Next, we split the meta-cohort into training (n=563) and validation (n=543) sets balanced for clinical risk factors. We identified 1012 genes whose expression was associated (P<0.01 log-likelihood test in Cox regression) with OS or DSS across the training set (Methods; Table 1; FIG. 1A; and FIG. 6). Of these genes, 343 (34%) were significant when only considering patients diagnosed with stage I tumors (n=394, 70%).

Prognostic Genes Reflect Distinct Tumor Microenvironment Programs.

Lung cancer tumor specimens generally contain multiple cell types in addition to the malignant epithelial cells, including stromal and infiltrating immune cells. We reasoned that individual prognostic genes might reflect distinct cell types and/or distinct processes related to tumor biology or the tumor microenvironment and therefore clustered the gene expression profiles of the 1012 prognostic genes within the training cohort using AutoSOME³⁰ (FIG. 1B). The four largest clusters were enriched for individual genes reflecting (1) proliferation and mitochondrial metabolic activity, (2) normal lung differentiation, (3) glycolytic metabolic activity and basal epithelial cell fate, and (4) immune-related functions. Kaplan-Meier curves stratifying survival based on the average expression levels of genes in each of these 4 clusters captured significant patterns of favorable (Clusters 2 and 4) and adverse (Clusters 1 and 3) prognostic association (FIG. 1C).

We compared these same 4 clusters to gene sets representing prior biological knowledge. Cluster 1, which contained many proliferation and cell-cycle related genes, was also highly enriched for a module of genes with higher expression in embryonic stem cells relative to more differentiated cell types and in genes highly expressed in less differentiated cancer histologies. Cluster 2 shared genes expressed more highly in differentiated cancer subtypes and in lung tracheal luminal cells, and high Cluster 2 expression was negatively associated with smoking status (Table 6), but was independently prognostic in multivariate analysis. Cluster 3 was enriched for genes distinguishing basal-like tumors from luminal-like adenocarcinoma of the breast. Cluster 3 also significantly overlapped signatures of lung tracheal basal cells, which contain airway stem cells. Finally, Cluster 4 most strongly overlapped with signature genes, including MS4A1 (CD20), characteristically expressed in B-cells as well as those expressed in lymph nodes and generically during immune responses. All overlaps reported here were significant (q<0.001) after correction for multiple hypothesis testing (Table 5).

TABLE 5
Selected significant overlaps between Autosome clusters of NSCLC prognostic genes, and curated gene sets.

| Auto- | | | | | | |
|---|---|---|---|---|---|---|
| some | | Cluster | Geneset | | | |
| cluster | Geneset | size | size | overlap | p | qval |
| 1 | SHEDDEN_LUNG_CANCER_POOR_SURVIVAL_A6 | 328 | 441 | 139 | 5.64E−156 | 9.46E−152 |
| 1 | ROSTY_CERVICAL_CANCER_PROLIFERATION_CLUSTER | 328 | 139 | 82 | 7.64E−118 | 1.83E−114 |
| 1 | RODRIGUES_THYROID_CARCINOMA_POORLY_DIFFERENTIATED_UP | 328 | 607 | 99 | 2.15E−77 | 1.82E−74 |
| 1 | WONG_EMBRYONIC_STEM_CELL_CORE | 328 | 332 | 77 | 7.99E−72 | 5.15E−69 |
| 1 | CELL_CYCLE_GO_0007049 | 328 | 311 | 46 | 2.80E−33 | 3.20E−30 |
| 2 | SHEDDEN_LUNG_CANCER_GOOD_SURVIVAL_A4 | 252 | 191 | 30 | 3.70E−26 | 4.72E−24 |
| 2 | RODRIGUES_THYROID_CARCINOMA_POORLY_DIFFERENTIATED_DN | 252 | 770 | 47 | 3.24E−22 | 3.52E−20 |
| 2 | SMID_BREAST_CANCER_BASAL_DN | 252 | 672 | 44 | 5.27E−22 | 5.66E−20 |
| 2 | Lung_sw | 252 | 110 | 8 | 2.67E−05 | 0.000657 |
| 3 | SHEDDEN_LUNG_CANCER_POOR_SURVIVAL_A6 | 97 | 441 | 26 | 6.36E−23 | 7.21E−21 |
| 3 | CHARAFE_BREAST_CANCER_LUMINAL_VS_BASAL_DN | 97 | 443 | 25 | 1.41E−21 | 1.48E−19 |
| 3 | RESPONSE_TO_EXTERNAL_STIMULUS | 97 | 312 | 12 | 6.99E−09 | 8.33E−07 |
| 4 | Lymphnode_sw | 39 | 235 | 14 | 9.28E−19 | 1.21E−16 |
| 4 | IMMUNE_RESPONSE | 39 | 235 | 10 | 4.45E−12 | 7.22E−10 |
| 4 | PASQUALUCCI_LYMPHOMA_BY_GC_STAGE_DN | 39 | 162 | 8 | 2.36E−10 | 1.20E−08 |
| 4 | HADDAD_B_LYMPHOCYTE_PROGENITOR | 39 | 279 | 8 | 1.71E−08 | 7.08E−07 |

TABLE 6
Cluster 2 (differentiation) membership is inversely
correlated with current or previous smoking status

| Cluster 2 | Ever smoker | Never smoker |
|---|---|---|
| High | 270 | 59 |
| Low | 185 | 106 |

P = 3 × 10⁻⁷ (Two-tailed Fisher exact test)
Cluster 1 is even more strongly (positively) associated, p = 4 × 10⁻⁹, cluster 3 less so (p = 0.005) and cluster 4 (immune) is not, p = 0.08.

A 9-Gene Molecular Prognostic Index for Non-Squamous NSCLC.

To construct a molecular prognostic index (MPI) combining distinct aspects of NSCLC biology, we selected the most prognostic genes from each of these 4 biologically defined clusters employing a multivariate approach to assess independent prognostic value as well as robustness in reproducibility analyses. The resulting model comprised 9 genes including 4 whose expression was associated with adverse risk—MAD2L1 (Mitotic arrest deficient 2-like 1), GINS1 (GINS complex subunit 1), SLC2A1 (Solute carrier family 2 facilitated glucose transporter, member 1; also known as GLUT-1), and KRT6A (keratin 6A)—as well as 5 genes whose expression was associated with favorable outcomes—TNIK (TRAF2 and NCK interacting kinase), BCAM (Basal cell adhesion molecule), KDM6A (lysine-specific demethylase 6A), FCGRT (Fc fragment of IgG receptor, alpha), and FAIM3 (Fas apoptotic inhibitory molecule 3).

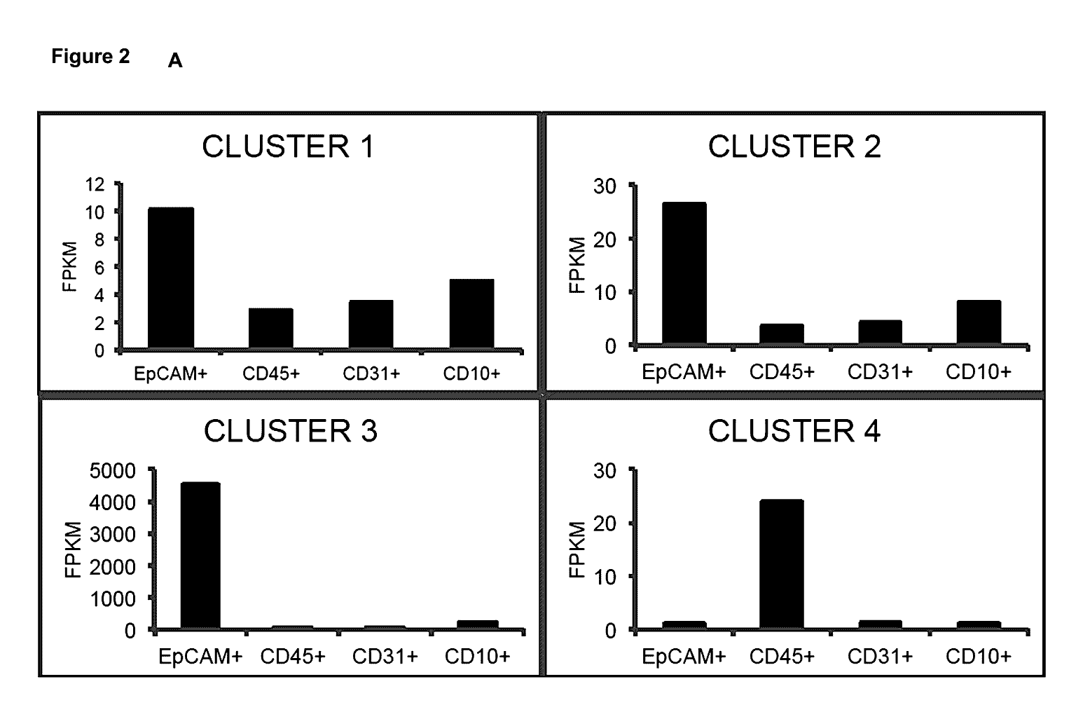

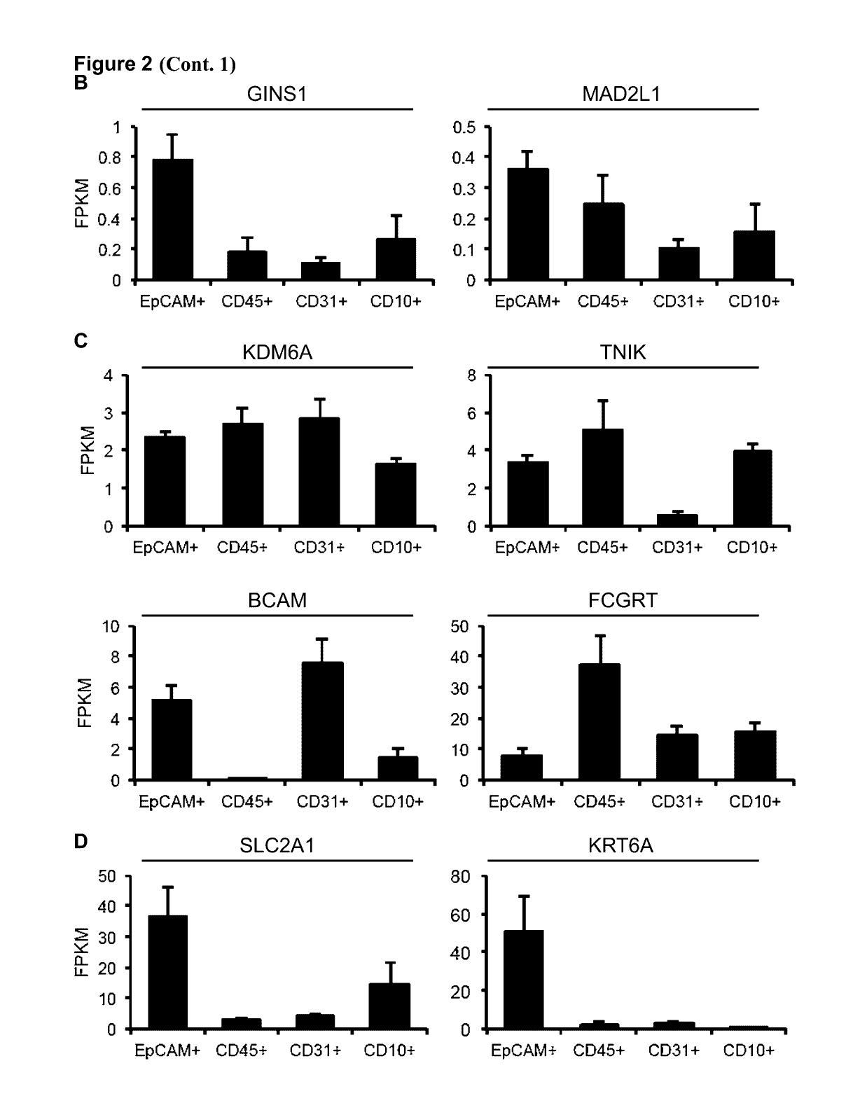

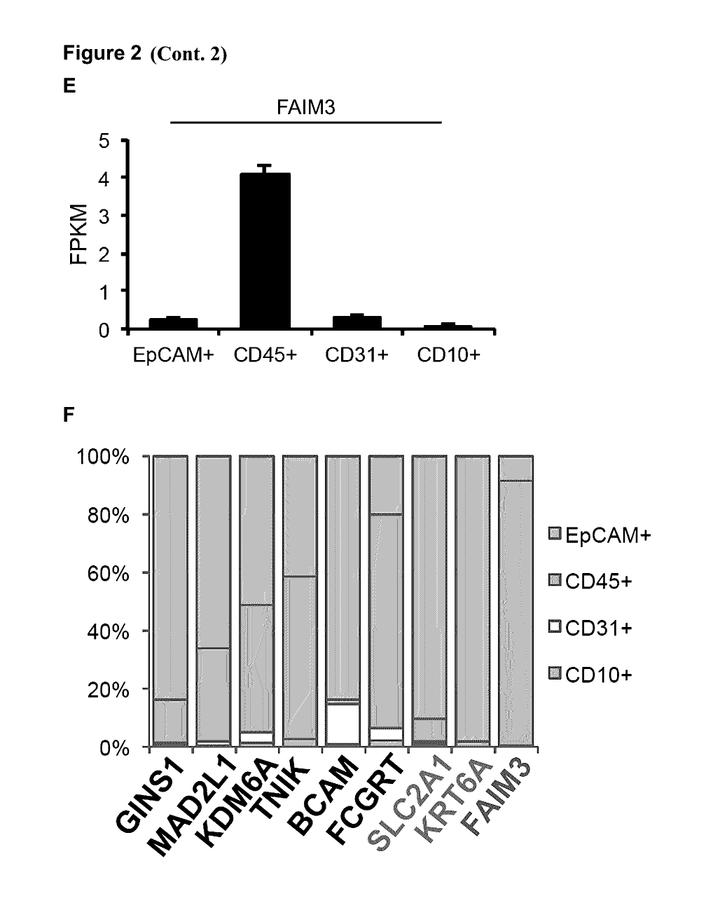

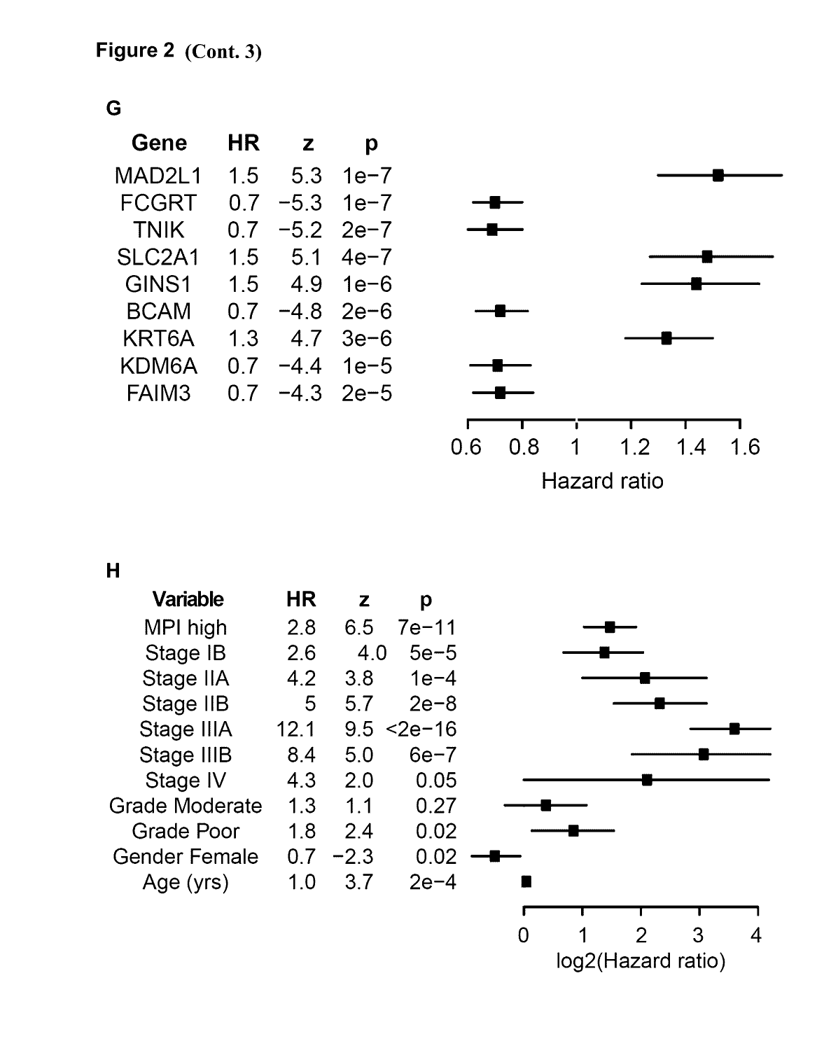

To assess how these genes were expressed across cell sub-populations, we performed whole transcriptome sequencing (RNA-seq) on freshly sorted populations of tumor cells from resected stage I lung adenocarcinomas (n=4; FIG. 2A-F). The genes from Clusters 1 and 3 displayed highest expression within EpCAM+ epithelial cells, as compared with CD45+ leukocytes, CD31+ endothelial cells, and CD10+ fibroblasts. In contrast, FAIM3 from Cluster 4 was expressed predominantly in leukocytes. The 4 genes from Cluster 2 were expressed variably within the sorted cell populations; these data reflect existing knowledge regarding the expression patterns of Cluster 2 genes (e.g., BCAM expression in epithelial and endothelial cells) and may explain the independent prognostic information provided by each gene within the 9-gene MPI. Altogether, for all 9 genes the vast majority of RNA transcripts found in tumors was derived from epithelial or immune cells (FIG. 2F). These findings confirm that the genes making up the MPI measure processes involving both tumor and stromal cells.

Based on the relative prognostic value in our microarray training cohort (FIG. 2G, FIG. 2H), we defined the MPI in terms of the expression levels of the 9 genes as:

MPI=4*MAD2L1+4*GINS1−5*FCGRT−5*TNIK−5*BCAM−5*KDM6A+5*SLC2A1+4*KRT6A−6*FAIM3

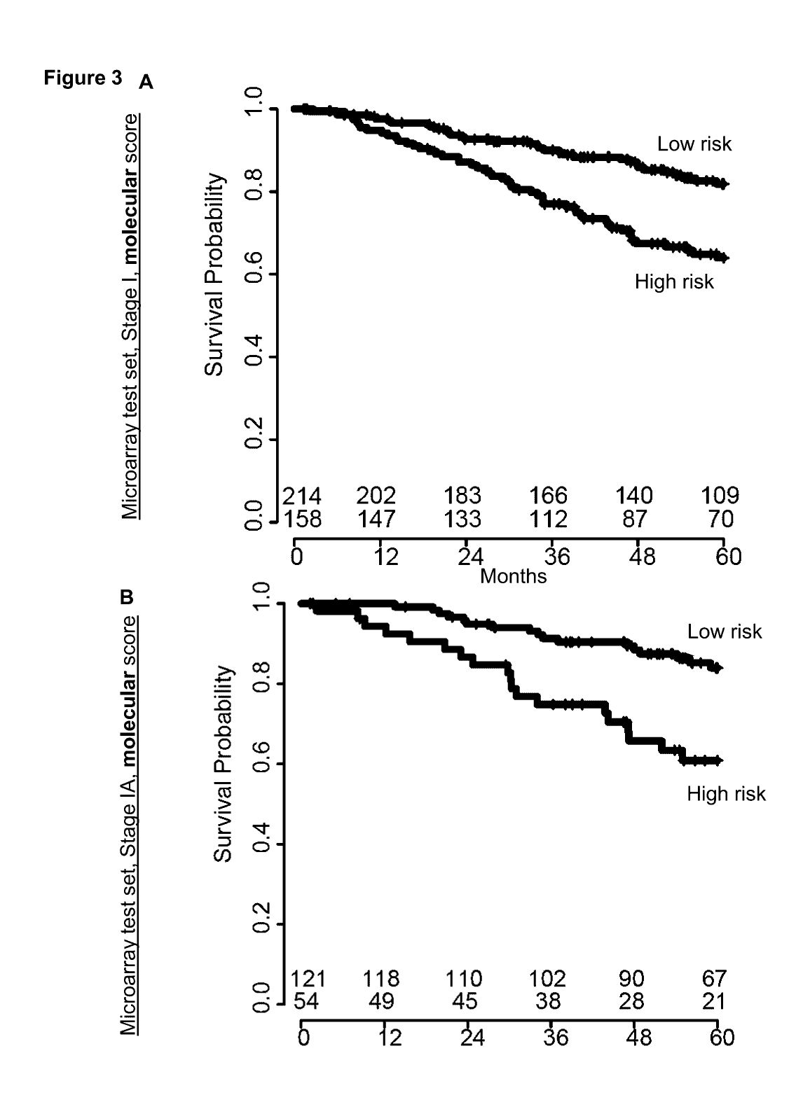

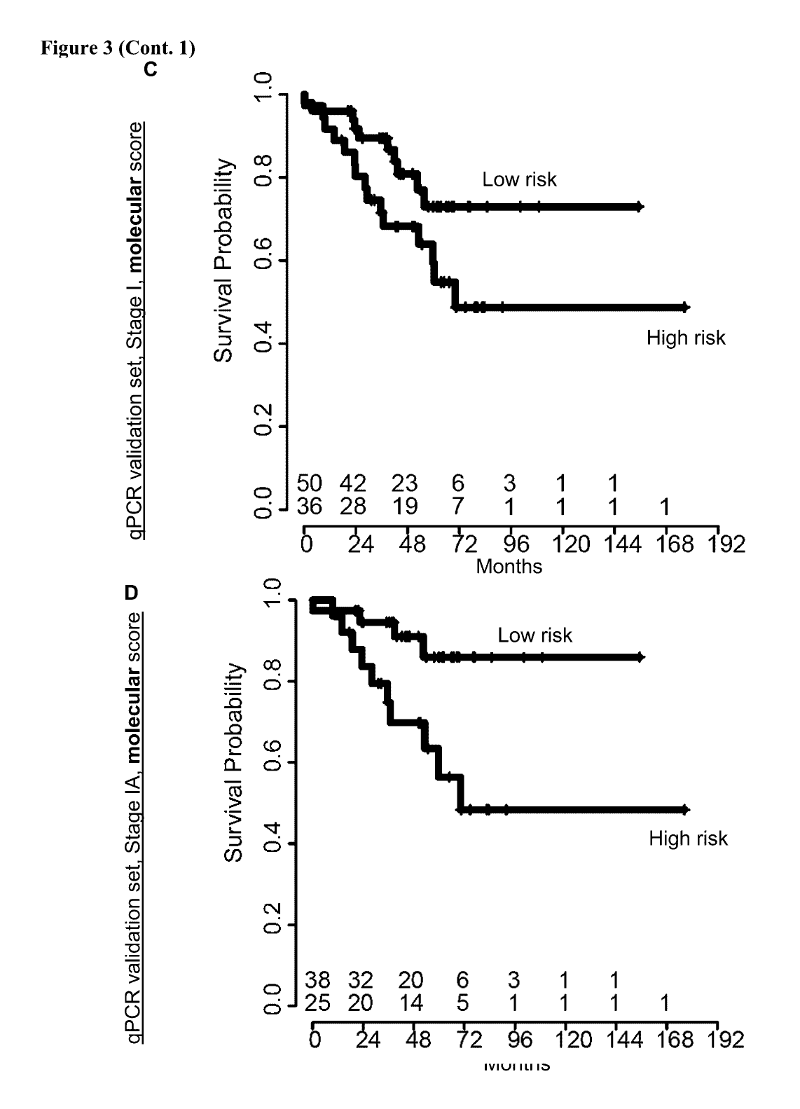

The MPI was strongly negatively associated with overall survival as a continuous score in the microarray training set. In the microarray training set the MPI ranged from −0.65 to +0.87 and each unit increase in its value was associated with a 11.8-fold increase in risk of death (95% CI 6.9-20.1; P<0.0001 log-likelihood test). In the validation set, the MPI range was from −0.58 to +1.07 (HR 7.7, 95% CI 4.4-13.3; P<0.0001). As a binary variable (high vs. low) with a cutoff set at the median MPI in the training set, the MPI was significantly associated with OS in both the training set (HR, 2.8, 95% CI 2.0-3.8; P<0.001, log-rank test) and the microarray validation set (HR, 2.4, 95% CI 1.8-3.3; P<0.001). In the training set the median survival time was 45 months for the high-risk MPI group vs. not-reached in the low risk group. The corresponding values were 48 months and not-reached for the validation set. The MPI stratification remained significant when considering only patients with stage I non-squamous NSCLC (FIG. 3A; HR, 2.3, 95% CI 1.5-3.5; P<0.001) of the validation set, and even within the subset of patients with stage IA disease (FIG. 3B; HR, 3.0, 95% CI 1.6-5.8; P<0.001), illustrating the ability of the MPI to identify high risk patients among those with the smallest tumors.

Validation of the MPI in Clinical Samples by qPCR.

Having confirmed the ability of the MPI to stratify risk among patients with stage I non-squamous NSCLC using microarrays and fresh tissues, we next sought to implement it as a qPCR assay that could be readily performed in clinical laboratories. We selected TaqMan assays for the 9 MPI genes as well as for 2 control housekeeping genes (AGPAT1 and PRPF40A). To confirm the ability of the qPCR-based MPI to stratify outcomes we validated it in a second independent cohort of 98 patients (summarized in Table 1). RNA was successfully purified from all 98 FFPE tissues up to 14 years old (median of 6 years old). The qPCR-based MPI successfully stratified high-risk and low-risk groups when applied to the FFPE validation cohort (P=0.03). Using the cut-off values for high and low risk defined in the microarray training set, the MPI separated high and low risk stage I (FIG. 3C; HR, 2.5, 95% CI 1.1-6.0; P=0.03) and stage IA patients (FIG. 3D; HR, 4.0, 95% CI 1.2-12.6; P=0.01). It also remained significant in multivariate analysis with basic clinical and pathological variables that were available in both the microarray and qPCR cohorts (age, gender, stage, and grade; Table 8). Taken together, these results confirm the performance of the 9-gene MPI as a prognostic tool in early stage non-squamous NSCLC, and suggest applicability for archived clinical samples.

TABLE 8
Multivariate of MPI in training, test and validation cohorts

| | Multivariate | | | | | |
|---|---|---|---|---|---|---|
| | Training array data | | Multivariate in test array data | | Multivariate in PCR cohort | |
| Variable | Hazard ratio (95% C.I.) | p | Hazard ratio (95% C.I.) | p | Hazard ratio (95% C.I.) | p |
| Molecular | 1.03 (1.02-1.04) | 9.00E−14 | 1.02 (0.98-1.01) | 2.00E−08 | 1.02 (1.00-1.04) | 0.02 |
| Stage IB | 2.44 (1.42-4.18) | 0.001 | 0.79 (1.27-0.47) | 0.36 | 2.41 (0.97-5.98) | 0.06 |
| Stage IIA | 4.34 (1.98-9.53) | 0.0003 | 1.88 (0.53-0.81) | 0.14 | 3.21 (1.06-9.70) | 0.04 |
| Stage IIB | 2.31 (1.20-4.42) | 0.01 | 1.96 (0.51-1.15) | 0.01 | 4.66 (1.00-21.80) | 0.05 |
| Stage IIIA | 6.31 (3.47-11.47) | 1.00E−09 | 3.38 (0.30-1.92) | 2.00E−05 | — | — |
| Stage IIIB | 7.38 (3.02-18.02) | 1.00E−05 | 3.24 (0.31-1.45) | 0.004 | — | — |
| Stage IV | 2.47 (0.56-10.90) | 0.23 | 18.51 (4.04-84.83) | 0.0002 | — | — |
| Grade Moderate | 0.74 (0.43-1.25) | 0.25 | 1.10 (0.91-0.59) | 0.76 | 1.11 (0.35-3.49) | 0.86 |
| Grade Poor | 0.61 (0.34-1.09) | 0.09 | 1.09 (0.92-0.56) | 0.81 | 1.15 (0.29-4.61) | 0.84 |
| Female | 1.08 (0.77-1.52) | 0.66 | 1.02 (0.98-0.73) | 0.9 | 0.62 (0.26-1.45) | 0.27 |
| Age (yrs) | 1.03 (1.01-1.05) | 0.001 | 1.03 (0.97-1.01) | 0.005 | 1.05 (1.01-1.09) | 0.02 |

One of the genes in the MPI, SLC2A1, encodes a major glucose transporter (GLUT-1) whose protein expression has previously been shown to correlate with uptake of fluorodeoxyglucose (FDG) by NSCLCs on positron emission tomography (PET) scans. We were therefore interested to compare how measurement of SLC2A1 mRNA using our method compared with measurement of GLUT-1 protein by immunohistochemistry (IHC) and FDG uptake on pre-treatment PET scans (FIG. 7). The expression of SLC2A1—as determined by qPCR—correlated strongly with GLUT-1 IHC staining levels (P<10⁻⁵) and to a lesser extent with tumor FDG maximum standardized uptake value (P<0.01). Thus, SLC2A1 transcript levels are correlated with expression and function of its protein, two tumor characteristics that have previously been shown to predict outcome

A Composite Molecular/Clinical Model for Predicting Survival in Non-Squamous NSCLC.

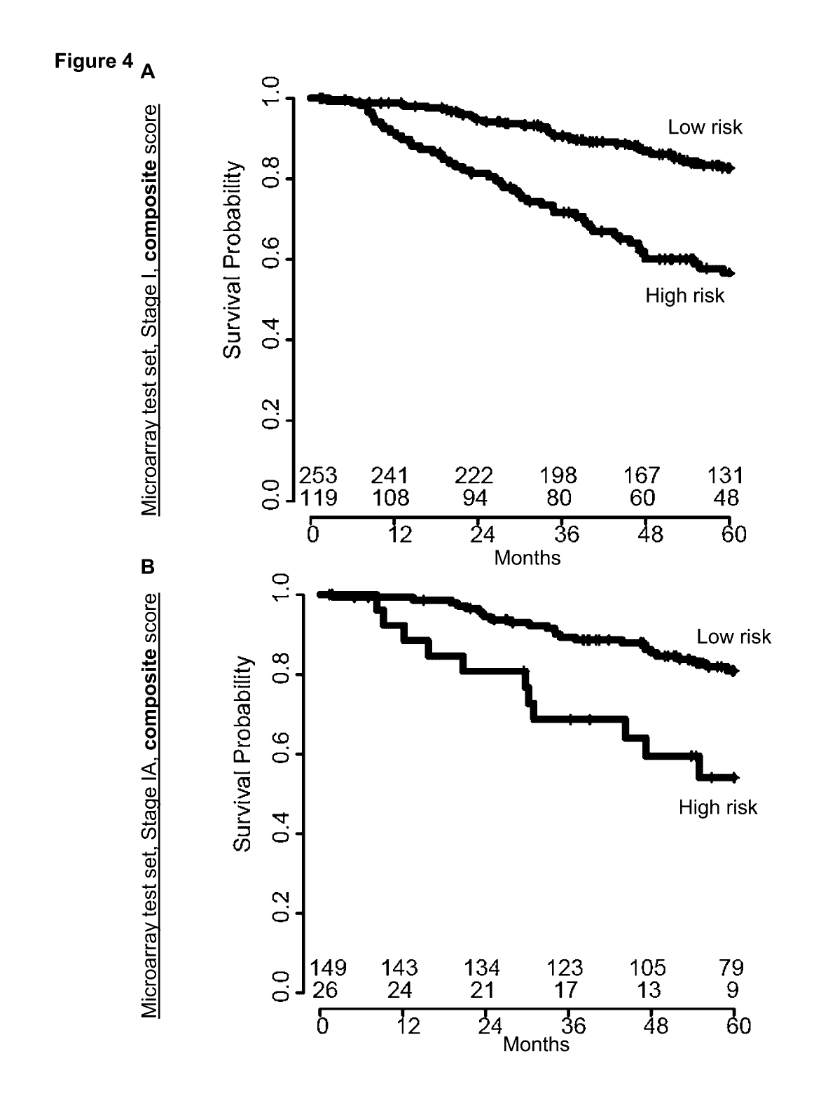

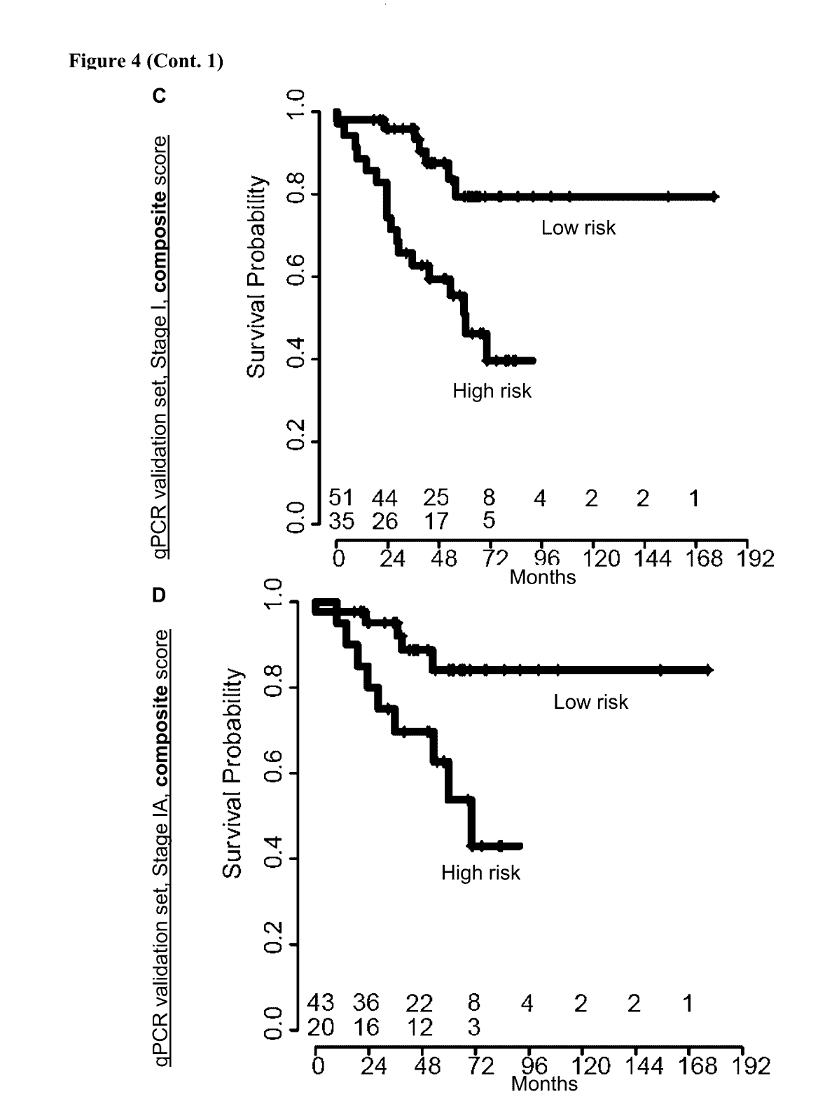

The MPI provided independent prognostic information when compared to standard clinicopathologic covariates that are readily assessable and were available in all of the cohorts. This suggested that combining the MPI with these covariates could create an even more robust risk index. For lung adenocarcinoma, previously proposed clinical prognostic factors include age, gender, and stage^{6,8,48-51}. To define relative weighting of these variables we utilized the Surveillance Epidemiology and End Results (SEER) database and determined hazard ratios for death within a large, diverse population. Over 28,000 patients with resected non-squamous NSCLC were identified. As expected, advanced age, male gender, and higher stage at diagnosis signified poor prognosis in this analysis. These factors were therefore combined into a clinical prognostic index (CPI), which was defined as the sum of the age, gender, and stage components from their coefficient (Table 3) in multivariate Cox regression (Methods). The CPI was significant in univariate analysis in the microarray and qPCR cohorts as well as in multivariate analysis incorporating the MPI (Table 2). We therefore combined the CPI with the MPI to form a composite risk model (CRM) defined in the training set (1.1*SEER+0.022*MPI), which successfully stratified patients by risk of death in the microarray and qPCR validation sets (FIG. 4; Table 2).

TABLE 2
Univariate and multivariate analysis of molecular, clinical,
and composite scores in training and validation cohorts.
SEER and composite SEER-MPI models

| | Microarray test set | | | qPCR Validation set | | |
|---|---|---|---|---|---|---|
| | Hazard ratio | | | Hazard ratio | | |
| Variable | (95% C.I.) | | p | (95% C.I.) | | p |
| Univariate | | | | | | |
| Molecular | 1.02 | (1.02-1.03) | 3.00E−13 | 1.02 | (1.00-1.03) | 0.01 |
| SEER (cts age) | 3.04 | (2.32-3.97) | 4.00E−16 | 6.10 | (1.78-20.71) | 0.004 |
| Multivariate | | | | | | |
| Molecular | 2.24 | (1.73-2.90) | 8.00E−10 | 2.47 | (1.33-4.60) | 0.004 |
| SEER (cts age) | 2.66 | (2.02-3.50) | 3.00E−12 | 5.92 | (1.85-19.00) | 0.003 |
| Composite MPI-SEER | 2.43 | (2.05-2.88) | <2e−16 | 2.83 | (1.62-4.95) | 0.0003 |
| (all stages) | | | | | | |
| Composite MPI-SEER | 3.34 | (2.44-4.58) | 6.00E−14 | 4.16 | (1.86-9.27) | 5.00E−04 |
| (high vs low, all stages) | | | | | | |
| Composite MPI-SEER | 2.4 | (1.8-3.2) | 4.00E−09 | 3.1 | (1.7-5.7) | 8.00E−05 |
| (stage 1) | | | | | | |
| Composite MPI-SEER | 3.2 | (2.1-4.9) | 3.00E−08 | 4.0 | (1.7-9.6) | 0.0008 |
| (high vs low, stage 1) | | | | | | |
| Composite MPI-SEER | 2.9 | (1.7-4.8) | 8.00E−05 | 4.8 | (1.9-12.2) | 0.00098 |
| (stage 1A) | | | | | | |
| Composite MPI-SEER | 3.1 | (1.5-6.2) | 0.001 | 3.8 | (1.3-11.5) | 0.01 |
| (high vs low, stage 1A) | | | | | | |

TABLE 3
Components of the SEER clinical prognostic model

| Variable | Cox coefficient |
|---|---|
| Gender Female vs Male | |
| Female vs male | −0.34 |
| Stage | |
| IB | 0.43 |
| IIA | 0.81 |
| IIB | 1.10 |
| IIIA | 1.20 |
| IIIB | 1.20 |
| Age | 0.024 × age (yrs) |

The CRM identified patients at higher risk of death across all stages, and when restricted to stage I of the qPCR validation cohort (FIG. 4C; HR, 4.0, 95% CI 1.7-9.6; P<0.001) or stage IA only (FIG. 4D; HR, 4.8, 95% CI 1.9-12.2; P<0.001). The CRM outperformed the MPI alone when assessed by Net Reclassification Improvement and Integrated Discrimination Improvement for assignment of risk of death by 5 years (Table 9). These data indicate that the CRM, which integrates both molecular and clinical risk-associated variables, provides more robust assessment of prognosis than the MPI alone.

TABLE 9
NRI and IDI analysis of 5 year survival. Net Reclassification Improvement (NRI) and
Integrated Discrimination Improvement (IDI) at 5 years for composite vs
molecular model fitted to training data

| | Training set | | Test set | | Validation set | |
|---|---|---|---|---|---|---|
| NRI at 5 years | Index | p | Index | p | Index | p |
| NRI for events | 0.16 | 3 × 10⁻² | 0.13 | 9 × 10⁻² | 0.15 | 0.38 |
| NRI for non-events | 0.38 | 2 × 10⁻¹⁴ | 0.40 | 4 × 10⁻¹⁵ | 0.42 | 0.0002 |
| Overall | 0.55 | 6 × 10⁻¹⁰ | 0.53 | 7 × 10⁻⁹ | 0.57 | 0.006 |
| IDI at 5 years | | | IDI | p | IDI | p |
| Increase for events (sensitivity) | 0.06 | | 0.06 | | 0.03 | |
| Decrease for non-events (specificity) | 0.03 | | 0.04 | | 0.06 | |
| IDI | 0.094 | 8 × 10⁻¹² | 0.095 | 4 × 10⁻⁹ | 0.09 | 0.005 |

TABLE 7
TaqMan gene expression assays used in this study.

| Gene ID | Purpose | TaqMan Assay ID | Assay Length (bp) |
|---|---|---|---|
| PRPF40A | Reference | Hs00215465_m1 | 73 |
| AGPAT1 | Reference | Hs00965850_g1 | 110 |
| SLC2A1 | Prognosis | Hs00892682_g1 | 76 |
| MAD2L1 | Prognosis | Hs01554515_g1 | 102 |
| TNIK | Prognosis | Hs01115830_m1 | 91 |
| BCAM | Prognosis | Hs01124111_g1 | 105 |
| FCGRT | Prognosis | Hs01108967_m1 | 107 |
| KDM6A | Prognosis | Hs00958902_m1 | 71 |
| FAIM3 | Prognosis | Custom (AJ89JRX) | 56 |
| KRT6A | Prognosis | Custom (AJBJW3Y) | 69 |
| GINS1 | Prognosis | Custom (AJCSU94) | 74 |

TABLE 8
autosome
Clusters

| AutoSOME | Gene |
|---|---|
| Cluster | Symbol |
| 1 | CDT1 |
| 1 | ASF1B |
| 1 | FAM64A |
| 1 | FEN1 |
| 1 | UBE2C |
| 1 | CCNA2 |
| 1 | CDCA8 |
| 1 | FOXM1 |
| 1 | GINS2 |
| 1 | KIF2C |
| 1 | KIF4A |
| 1 | MCM10 |
| 1 | TPX2 |
| 1 | BIRC5 |
| 1 | CCNB1 |
| 1 | CCNB2 |
| 1 | CDC20 |
| 1 | CENPA |
| 1 | E2F8 |
| 1 | ESPL1 |
| 1 | EXO1 |
| 1 | HJURP |
| 1 | KIF20A |
| 1 | MCM4 |
| 1 | MKI67 |
| 1 | MLF1IP |
| 1 | NCAPG2 |
| 1 | ORC6 |
| 1 | RAD54L |
| 1 | TOP2A |
| 1 | TYMS |
| 1 | ZWINT |
| 1 | CDC45 |
| 1 | CDC6 |
| 1 | CENPF |
| 1 | DTL |
| 1 | KIF15 |
| 1 | KIFC1 |
| 1 | MELK |
| 1 | NCAPG |
| 1 | NUSAP1 |
| 1 | PRC1 |
| 1 | RACGAP1 |
| 1 | SPAG5 |
| 1 | TK1 |
| 1 | BUB1B |
| 1 | CDCA3 |
| 1 | CDKN3 |
| 1 | CEP55 |
| 1 | CHEK1 |
| 1 | CKS2 |
| 1 | DLGAP5 |
| 1 | ERCC6L |
| 1 | HMMR |
| 1 | KIF11 |
| 1 | KIF14 |
| 1 | MAD2L1 |
| 1 | MYBL2 |
| 1 | PBK |
| 1 | POLE2 |
| 1 | RECQL4 |
| 1 | RRM2 |
| 1 | TIMELESS |
| 1 | ASPM |
| 1 | BUB1 |
| 1 | CDC25C |
| 1 | E2F1 |
| 1 | GINS1 |
| 1 | H2AFZ |
| 1 | KIAA0101 |
| 1 | NCAPH |
| 1 | NEK2 |
| 1 | AURKB |
| 1 | CHAF1A |
| 1 | KIF23 |
| 1 | MCM7 |
| 1 | NDC80 |
| 1 | PLK1 |
| 1 | SPC25 |
| 1 | TRIP13 |
| 1 | TROAP |
| 1 | BRCA1 |
| 1 | FANCI |
| 1 | KPNA2 |
| 1 | MCM2 |
| 1 | MCM6 |
| 1 | OIP5 |
| 1 | POLQ |
| 1 | SHCBP1 |
| 1 | TTK |
| 1 | CCNE2 |
| 1 | PCNA |
| 1 | DSCC1 |
| 1 | H2AFX |
| 1 | PLK4 |
| 1 | CENPN |
| 1 | RFC4 |
| 1 | BLM |
| 1 | LMNB2 |
| 1 | TUBA1C |
| 1 | DTYMK |
| 1 | MRPL11 |
| 1 | NEIL3 |
| 1 | HMGB2 |
| 1 | PAICS |
| 1 | RFC5 |
| 1 | TMEM48 |
| 1 | MSH6 |
| 1 | SSRP1 |
| 1 | DKC1 |
| 1 | CDCA4 |
| 1 | CKAP5 |
| 1 | CSE1L |
| 1 | MTHFD1 |
| 1 | MZT2A |
| 1 | PSAT1 |
| 1 | RAD51 |
| 1 | RRM1 |
| 1 | TOPBP1 |
| 1 | ZWILCH |
| 1 | GEMIN6 |
| 1 | NCAPD2 |
| 1 | SNRPB |
| 1 | TMEM194A |
| 1 | MSH2 |
| 1 | SNRPC |
| 1 | ATP5J2 |
| 1 | CHCHD3 |
| 1 | RDBP |
| 1 | UNG |
| 1 | AIMP2 |
| 1 | BYSL |
| 1 | NOP56 |
| 1 | SLBP |
| 1 | SNRPF |
| 1 | WDR76 |
| 1 | CTPS |
| 1 | MRPS12 |
| 1 | NME1 |
| 1 | NUP155 |
| 1 | RFC2 |
| 1 | TUBB |
| 1 | BRIP1 |
| 1 | RPL39L |
| 1 | SMC2 |
| 1 | EIF4A3 |
| 1 | ILF2 |
| 1 | RFC3 |
| 1 | ZNF259 |
| 1 | CCDC86 |
| 1 | CCT7 |
| 1 | GPSM2 |
| 1 | MTHFD2 |
| 1 | PSMD12 |
| 1 | PTRH2 |
| 1 | MRPS17 |
| 1 | MRPS30 |
| 1 | PNO1 |
| 1 | RPA3 |
| 1 | GART |
| 1 | GPN1 |
| 1 | IGF2BP3 |
| 1 | MRPS16 |
| 1 | PPM1G |
| 1 | PRPF4 |
| 1 | RPP40 |
| 1 | TFAM |
| 1 | TUBA1B |
| 1 | ATIC |
| 1 | GAR1 |
| 1 | SIP1 |
| 1 | GLRX3 |
| 1 | HMBS |
| 1 | NUP37 |
| 1 | CSTF2 |
| 1 | BRCA2 |
| 1 | EEF1E1 |
| 1 | MRPL42 |
| 1 | MYL6B |
| 1 | NOP16 |
| 1 | POLE3 |
| 1 | PSMB7 |
| 1 | TRIB3 |
| 1 | CHCHD2 |
| 1 | POLR2H |
| 1 | ACP1 |
| 1 | CTSL2 |
| 1 | HPRT1 |
| 1 | MRPL17 |
| 1 | TIMM8B |
| 1 | TSSC1 |
| 1 | HSPE1 |
| 1 | TACO1 |
| 1 | DENR |
| 1 | MRPS7 |
| 1 | PXMP2 |
| 1 | SFXN1 |
| 1 | ATP5G3 |
| 1 | MOCOS |
| 1 | RUVBL1 |
| 1 | SSBP1 |
| 1 | CDK4 |
| 1 | CYC1 |
| 1 | GPN3 |
| 1 | TFDP1 |
| 1 | NRAS |
| 1 | CCT4 |
| 1 | CCT5 |
| 1 | COQ3 |
| 1 | COX5B |
| 1 | GLRX2 |
| 1 | PSMD14 |
| 1 | ARPC1A |
| 1 | GGH |
| 1 | METTL5 |
| 1 | MRPS35 |
| 1 | YKT6 |
| 1 | COX7B |
| 1 | EMG1 |
| 1 | GPD2 |
| 1 | HSPA4 |
| 1 | POLD2 |
| 1 | PSMA5 |
| 1 | TIMM17A |
| 1 | ALG3 |
| 1 | COPS8 |
| 1 | DDX21 |
| 1 | NBN |
| 1 | NCBP1 |
| 1 | SRSF9 |
| 1 | TMEM93 |
| 1 | UMPS |
| 1 | EIF2B1 |
| 1 | NDUFA8 |
| 1 | PSMD1 |
| 1 | SENP2 |
| 1 | TPI1 |
| 1 | TRIAP1 |
| 1 | ZC3H15 |
| 1 | C1QBP |
| 1 | MDH1 |
| 1 | MDH2 |
| 1 | MTL5 |
| 1 | PSMA6 |
| 1 | PSME3 |
| 1 | ATP5B |
| 1 | CCDC51 |
| 1 | FASTKD2 |
| 1 | GARS |
| 1 | GOLT1B |
| 1 | PWP1 |
| 1 | SLC25A3 |
| 1 | VANGL1 |
| 1 | GRPEL1 |
| 1 | HNRNPAB |
| 1 | STRAP |
| 1 | CHML |
| 1 | MIF |
| 1 | NAA50 |
| 1 | NDUFS1 |
| 1 | COX5A |
| 1 | DERA |
| 1 | HMGA1 |
| 1 | COX7A2L |
| 1 | DDX1 |
| 1 | ETFA |
| 1 | KIAA0020 |
| 1 | NDUFB4 |
| 1 | TSFM |
| 1 | WDR43 |
| 1 | NIT2 |
| 1 | UCK2 |
| 1 | MRPS22 |
| 1 | PHKA1 |
| 1 | UCHL3 |
| 1 | YWHAZ |
| 1 | GLRX5 |
| 1 | RRP1 |
| 1 | TOMM70A |
| 1 | TRMT61B |
| 1 | DLD |
| 1 | DRG1 |
| 1 | DYNLL1 |
| 1 | FAM162A |
| 1 | GNG4 |
| 1 | PSMA2 |
| 1 | TMEM22 |
| 1 | ACOT7 |
| 1 | B4GALT5 |
| 1 | ERLIN1 |
| 1 | NUDT15 |
| 1 | EIF4G1 |
| 1 | GAL |
| 1 | GALNT14 |
| 1 | POMP |
| 1 | PTS |
| 1 | SUPT16H |
| 1 | XPO1 |
| 1 | CDC27 |
| 1 | GPI |
| 1 | LRP8 |
| 1 | MCFD2 |
| 1 | PFN2 |
| 1 | PPP4C |
| 1 | TCP1 |
| 1 | ADSL |
| 1 | PDE6D |
| 1 | CARHSP1 |
| 1 | CDK5 |
| 1 | PCMT1 |
| 1 | PDIA6 |
| 1 | RGS20 |
| 1 | RNASEH1 |
| 1 | RTCD1 |
| 1 | PDHX |
| 1 | TM9SF4 |
| 1 | ABCE1 |
| 1 | DNTTIP2 |
| 1 | HBXIP |
| 1 | TSN |
| 1 | DUSP11 |
| 1 | SNAPC1 |
| 1 | UGGT1 |
| 1 | DARS |
| 1 | NAA10 |
| 1 | PAFAH1B2 |
| 1 | EXOSC9 |
| 1 | MFI2 |
| 2 | GPD1L |
| 2 | SELENBP1 |
| 2 | TMPRSS2 |
| 2 | SCTR |
| 2 | ADCY9 |
| 2 | AK1 |
| 2 | CYP2B7P1 |
| 2 | PPP2R5A |
| 2 | ATP8A1 |
| 2 | CTSH |
| 2 | DRAM1 |
| 2 | HLF |
| 2 | SLC27A3 |
| 2 | GPR116 |
| 2 | IL6R |
| 2 | NR3C2 |
| 2 | PLA2G1B |
| 2 | RHOBTB2 |
| 2 | ST3GAL5 |
| 2 | CD302 |
| 2 | DLC1 |
| 2 | FAM149A |
| 2 | LIMCH1 |
| 2 | MBIP |
| 2 | STXBP1 |
| 2 | UNC13B |
| 2 | CYP4B1 |
| 2 | NEDD4L |
| 2 | PBXIP1 |
| 2 | STEAP4 |
| 2 | ABCC6 |
| 2 | APOH |
| 2 | MALL |
| 2 | NINJ2 |
| 2 | PIGR |
| 2 | PTPN13 |
| 2 | HNF1B |
| 2 | NPC2 |
| 2 | CAT |
| 2 | COL4A3 |
| 2 | FYCO1 |
| 2 | PER3 |
| 2 | CPM |
| 2 | ECHDC2 |
| 2 | RGN |
| 2 | SEPW1 |
| 2 | PDPK1 |
| 2 | FAM117A |
| 2 | MYLIP |
| 2 | SLC47A1 |
| 2 | CRYM |
| 2 | FBP1 |
| 2 | GDF15 |
| 2 | FBXO38 |
| 2 | COL4A4 |
| 2 | SYNE1 |
| 2 | ASAP3 |
| 2 | DHRS7B |
| 2 | DPYSL2 |
| 2 | FCGRT |
| 2 | HABP2 |
| 2 | CIT |
| 2 | DOK4 |
| 2 | ENPP4 |
| 2 | RALGPS1 |
| 2 | TLE2 |
| 2 | ABAT |
| 2 | KIAA0494 |
| 2 | IRX5 |
| 2 | TRAK2 |
| 2 | DAAM2 |
| 2 | DNALI1 |
| 2 | LASS4 |
| 2 | LRIG1 |
| 2 | PPFIBP2 |
| 2 | TMEM80 |
| 2 | CIRBP |
| 2 | CLU |
| 2 | RBPMS |
| 2 | RFTN1 |
| 2 | SPOP |
| 2 | TRIM2 |
| 2 | CAPN3 |
| 2 | TMEM87A |
| 2 | LMF1 |
| 2 | NEIL1 |
| 2 | TENC1 |
| 2 | CAMK1D |
| 2 | CAMTA1 |
| 2 | RCAN2 |
| 2 | SERPIND1 |
| 2 | CMAH |
| 2 | CYFIP2 |
| 2 | IL11RA |
| 2 | MTUS1 |
| 2 | UBL3 |
| 2 | CEACAM6 |
| 2 | FAAH |
| 2 | REEP5 |
| 2 | SPINK5 |
| 2 | TERF2IP |
| 2 | EFNA1 |
| 2 | VPS13D |
| 2 | HIP1 |
| 2 | SARM1 |
| 2 | ZNF238 |
| 2 | CCDC28A |
| 2 | SCP2 |
| 2 | SCPEP1 |
| 2 | CELF2 |
| 2 | FCGBP |
| 2 | ARHGEF2 |
| 2 | CHKA |
| 2 | EPB41L5 |
| 2 | GLS |
| 2 | HPN |
| 2 | KIAA0513 |
| 2 | TSPAN12 |
| 2 | CST3 |
| 2 | TMEM59 |
| 2 | ATP1A2 |
| 2 | MMP15 |
| 2 | PURA |
| 2 | GPX4 |
| 2 | PRSS8 |
| 2 | ALDH9A1 |
| 2 | BZRAP1 |
| 2 | CD1E |
| 2 | GPR172B |
| 2 | SLC1A7 |
| 2 | SORL1 |
| 2 | AKAP13 |
| 2 | BSDC1 |
| 2 | CRBN |
| 2 | IL6ST |
| 2 | MAGI2 |
| 2 | TMEM63A |
| 2 | CCPG1 |
| 2 | TOX3 |
| 2 | TRAM1 |
| 2 | USP47 |
| 2 | YPEL5 |
| 2 | ZC3H7A |
| 2 | ACACB |
| 2 | ANKHD1 |
| 2 | CACNB1 |
| 2 | DEPTOR |
| 2 | FAM129A |
| 2 | PLEKHB1 |
| 2 | PREPL |
| 2 | TMEM159 |
| 2 | CLK4 |
| 2 | GPRASP1 |
| 2 | MECOM |
| 2 | PDK2 |
| 2 | SYF2 |
| 2 | CBX7 |
| 2 | CHD9 |
| 2 | HNMT |
| 2 | NISCH |
| 2 | ATP1B1 |
| 2 | B3GALT2 |
| 2 | CDC42BPA |
| 2 | EZH1 |
| 2 | KBTBD10 |
| 2 | NRXN3 |
| 2 | COL9A2 |
| 2 | RCOR3 |
| 2 | SULT1C2 |
| 2 | FARP2 |
| 2 | INPP5B |
| 2 | ANGPT1 |
| 2 | MAOB |
| 2 | PRDM2 |
| 2 | RPS6KA5 |
| 2 | AMT |
| 2 | ANKRA2 |
| 2 | RBM22 |
| 2 | RPS6KA3 |
| 2 | TMEM57 |
| 2 | DOCK1 |
| 2 | IKBKB |
| 2 | MACF1 |
| 2 | RAB11FIP1 |
| 2 | RAPGEF3 |
| 2 | SCYL3 |
| 2 | TNIK |
| 2 | FAM172A |
| 2 | KIAA0754 |
| 2 | PI4KA |
| 2 | RETN |
| 2 | USP4 |
| 2 | VPS13C |
| 2 | CMPK1 |
| 2 | LUC7L |
| 2 | NFIB |
| 2 | OXR1 |
| 2 | SLTM |
| 2 | ETV1 |
| 2 | LYST |
| 2 | TMBIM4 |
| 2 | TXNIP |
| 2 | EPHA3 |
| 2 | GNPTAB |
| 2 | IGSF9B |
| 2 | CX3CL1 |
| 2 | CSAD |
| 2 | MEF2C |
| 2 | UBE2G2 |
| 2 | ZBTB48 |
| 2 | FOXN3 |
| 2 | GOLGA2B |
| 2 | IL1F7 |
| 2 | PPOX |
| 2 | CCNL2 |
| 2 | FAM164A |
| 2 | HERC1 |
| 2 | MAP3K3 |
| 2 | SLC18A2 |
| 2 | ZNF264 |
| 2 | CNR1 |
| 2 | PPP1R12B |
| 2 | GSDMB |
| 2 | NXF1 |
| 2 | RNF19A |
| 2 | ARHGEF9 |
| 2 | SETD6 |
| 2 | ZFHX3 |
| 2 | ZNF20 |
| 2 | GALNT12 |
| 2 | KDM6A |
| 2 | PCM1 |
| 2 | ZNF552 |
| 2 | ZRSR2 |
| 2 | BCAM |
| 2 | CDC14B |
| 2 | CREBL2 |
| 2 | DUSP8 |
| 2 | PIK3IP1 |
| 2 | ARMCX3 |
| 2 | SOBP |
| 2 | ZNF821 |
| 2 | BTG2 |
| 2 | PSD3 |
| 2 | TSPYL2 |
| 2 | MTMR9 |
| 2 | ADRA2A |
| 2 | TLE4 |
| 2 | SPINK1 |
| 2 | TTC15 |
| 2 | CBX6 |
| 2 | LETMD1 |
| 3 | ARNTL2 |
| 3 | KRT6A |
| 3 | SLC16A3 |
| 3 | PLAUR |
| 3 | STEAP1 |
| 3 | AHNAK2 |
| 3 | COL7A1 |
| 3 | FSCN1 |
| 3 | SLC2A1 |
| 3 | PFKP |
| 3 | BCAR3 |
| 3 | BID |
| 3 | FHL2 |
| 3 | SMOX |
| 3 | ADM |
| 3 | TMSB10 |
| 3 | S100A9 |
| 3 | CDCP1 |
| 3 | LRFN4 |
| 3 | LY6D |
| 3 | PLEK2 |
| 3 | RHOC |
| 3 | S100A8 |
| 3 | TUBB3 |
| 3 | LAMC2 |
| 3 | TGM2 |
| 3 | CALU |
| 3 | RHOF |
| 3 | SERPINB5 |
| 3 | STX1A |
| 3 | TUBA4A |
| 3 | DSC2 |
| 3 | FOSL1 |
| 3 | CORO1C |
| 3 | CTSL1 |
| 3 | MAP4K4 |
| 3 | RASAL2 |
| 3 | RND3 |
| 3 | SLC16A1 |
| 3 | TGFBI |
| 3 | CSTB |
| 3 | TSKU |
| 3 | ERO1L |
| 3 | MYO1E |
| 3 | VEGFC |
| 3 | KLK6 |
| 3 | OAS1 |
| 3 | PLIN2 |
| 3 | ACTR3 |
| 3 | GSTO1 |
| 3 | PKP2 |
| 3 | SLC25A37 |
| 3 | DKK1 |
| 3 | PKM2 |
| 3 | PPARD |
| 3 | S100A11 |
| 3 | AP2S1 |
| 3 | ATP1B3 |
| 3 | MT2A |
| 3 | SNAI1 |
| 3 | ANXA2 |
| 3 | PDXK |
| 3 | SEC23A |
| 3 | STC1 |
| 3 | STK24 |
| 3 | TRIM29 |
| 3 | KYNU |
| 3 | PYGL |
| 3 | RALA |
| 3 | ST3GAL4 |
| 3 | DCBLD2 |
| 3 | PITX1 |
| 3 | PLOD2 |
| 3 | ZNF185 |
| 3 | HK2 |
| 3 | PGK1 |
| 3 | SQRDL |
| 3 | STC2 |
| 3 | ANXA2P1 |
| 3 | AP2M1 |
| 3 | OAS3 |
| 3 | S100A10 |
| 3 | S100A12 |
| 3 | P4HA1 |
| 3 | TEAD4 |
| 3 | SEC61G |
| 3 | CKAP4 |
| 3 | GMFB |
| 3 | NMI |
| 3 | SEMA3C |
| 3 | ALDOA |
| 3 | HTR3A |
| 3 | WWTR1 |
| 3 | ANKRD57 |
| 3 | MAP2K1 |
| 3 | PVR |
| 3 | TGIF1 |
| 4 | ARHGAP25 |
| 4 | LTB |
| 4 | CCL19 |
| 4 | FAIM3 |
| 4 | IL16 |
| 4 | PRKCB |
| 4 | TRAF3IP3 |
| 4 | STAP1 |
| 4 | MS4A1 |
| 4 | CD37 |
| 4 | IL7R |
| 4 | BANK1 |
| 4 | ICAM3 |
| 4 | PTPRCAP |
| 4 | SLAMF1 |
| 4 | BLNK |
| 4 | RASGRP2 |
| 4 | CD22 |
| 4 | CD79A |
| 4 | DENND1C |
| 4 | ZAP70 |
| 4 | IGJ |
| 4 | LAX1 |
| 4 | FCRL2 |
| 4 | CR2 |
| 4 | FAM65B |
| 4 | HERPUD1 |
| 4 | FYN |
| 4 | IRF4 |
| 4 | FAM46C |
| 4 | HECA |
| 4 | HMHA1 |
| 4 | MAN2B1 |
| 4 | P2RY13 |
| 4 | PTK2B |
| 4 | PTGDS |
| 4 | CLEC2D |
| 4 | GFI1 |
| 4 | TNFRSF13B |
| 5 | LUC7L3 |
| 5 | HNRNPA3 |
| 5 | SFRS18 |
| 5 | RUFY3 |
| 5 | CLK1 |
| 5 | NKTR |
| 5 | S100PBP |
| 5 | GOLGA8A |
| 5 | OGT |
| 5 | SAFB2 |
| 5 | ZNF266 |
| 5 | RBM6 |
| 5 | TSC1 |
| 5 | ZNF573 |
| 5 | RBM5 |
| 5 | SENP7 |
| 5 | ZNF83 |
| 5 | ZNF682 |
| 5 | ZNF14 |
| 5 | ATRX |
| 5 | ZNF280D |
| 5 | USP48 |
| 5 | CDADC1 |
| 5 | ZNF211 |
| 5 | MGEA5 |
| 5 | GNRH1 |
| 5 | KIAA1009 |
| 5 | PCMTD2 |
| 5 | PILRB |
| 5 | ATF7IP2 |
| 5 | PPM1B |
| 5 | MPDZ |
| 5 | RNF38 |
| 6 | NDUFA9 |
| 6 | PHB2 |
| 6 | TFG |
| 6 | GCLC |
| 6 | STAMBP |
| 6 | PSMD7 |
| 6 | ABCF2 |
| 6 | COX7A2 |
| 6 | LTBR |
| 6 | TXNDC9 |
| 6 | AGFG1 |
| 6 | COPB2 |
| 6 | GCLM |
| 6 | ABCC2 |
| 6 | PPP3R1 |
| 6 | TXN |
| 6 | TARBP2 |
| 6 | ACOT13 |
| 6 | TTLL12 |
| 6 | IDH1 |
| 7 | BTG4 |
| 7 | PRTN3 |
| 7 | LGALS14 |
| 7 | NAA11 |
| 7 | DMRT1 |
| 7 | FABP7 |
| 7 | KIR2DL1 |
| 7 | ZNF343 |
| 7 | ATOH1 |
| 7 | RIMS2 |
| 7 | NEBL |
| 7 | CIDEA |
| 7 | DUSP13 |
| 7 | TUBB4Q |
| 7 | ZNF8 |
| 7 | HOXA11 |
| 7 | CDS2 |
| 8 | ITIH5 |
| 8 | MFAP4 |
| 8 | FMO3 |
| 8 | MEOX2 |
| 8 | FRZB |
| 8 | FBLN5 |
| 8 | LIMS2 |
| 8 | RUNX1T1 |
| 8 | LMOD1 |
| 8 | FOXF2 |
| 8 | OGN |
| 8 | ELN |
| 8 | LBH |
| 8 | FILIP1L |
| 9 | AHSA1 |
| 9 | CINP |
| 9 | EIF2B2 |
| 9 | NSDHL |
| 9 | LRRC61 |
| 9 | SIVA1 |
| 9 | DNPEP |
| 9 | KARS |
| 9 | UBL4A |
| 9 | STYXL1 |
| 9 | SLC3A2 |
| 9 | TPD52L2 |
| 10 | CSHL1 |
| 10 | SLC17A1 |
| 10 | DRD3 |
| 10 | GABRA6 |
| 10 | KRT35 |
| 10 | MADCAM1 |
| 10 | ACVR1B |
| 10 | CHRNA1 |
| 10 | RAB11B |
| 11 | KIF1C |
| 11 | SSBP3 |
| 11 | POLH |
| 11 | HERC2 |
| 11 | LGR4 |
| 11 | SLC11A2 |
| 11 | GSTT1 |
| 12 | SLC2A3 |
| 12 | TUBB6 |
| 12 | IL6 |
| 12 | MCAM |
| 12 | CXCR7 |
| 12 | ADCY3 |
| 12 | CSDA |
| 13 | HDAC2 |
| 13 | DPM1 |
| 13 | PCID2 |
| 13 | DUT |
| 13 | HEBP2 |
| 13 | CPOX |
| 14 | AKR1C1 |
| 14 | AKR1C2 |
| 14 | FURIN |
| 14 | FGG |
| 14 | NMB |
| 14 | INHA |
| 15 | USP20 |
| 15 | POU6F1 |
| 15 | TAF1C |
| 15 | TYK2 |
| 15 | PPP6R2 |
| 15 | SPG7 |

### REFERENCES

1. Siegel R, Naishadham D, Jemal A: Cancer statistics, 2013. CA Cancer J Clin 63:11-30, 2013
2. Reed M F, Molloy M, Dalton E L, et al: Survival after resection for lung cancer is the outcome that matters. American journal of surgery 188:598-602, 2004
3. Goldstraw P, Crowley J, Chansky K, et al: The IASLC Lung Cancer Staging Project: proposals for the revision of the TNM stage groupings in the forthcoming (seventh) edition of the TNM Classification of malignant tumours. Journal of thoracic oncology: official publication of the International Association for the Study of Lung Cancer 2:706-14, 2007
4. Sangha R, Price J, Butts C A: Adjuvant therapy in non-small cell lung cancer: current and future directions. The oncologist 15:862-72, 2010
5. Pignon J P, Tribodet H, Scagliotti G V, et al: Lung adjuvant cisplatin evaluation: a pooled analysis by the LACE Collaborative Group. J Clin Oncol 26:3552-9, 2008
6. Harpole D H, Jr., Herndon J E, 2nd, Young W G, Jr., et al: Stage I nonsmall cell lung cancer. A multivariate analysis of treatment methods and patterns of recurrence. Cancer 76:787-96, 1995
7. Yovino S, Kwok Y, Krasna M, et al: An association between preoperative anemia and decreased survival in early-stage non-small-cell lung cancer patients treated with surgery alone. Int J Radiat Oncol Biol Phys 62:1438-43, 2005
8. Chang M Y, Mentzer S J, Colson Y L, et al: Factors predicting poor survival after resection of stage IA non-small cell lung cancer. J Thorac Cardiovasc Surg 134:850-6, 2007
9. Horne Z D, Jack R, Gray Z T, et al: Increased levels of tumor-infiltrating lymphocytes are associated with improved recurrence-free survival in stage 1A non-small-cell lung cancer. J Surg Res 171:1-5, 2011
10. Carr S R, Schuchert M J, Pennathur A, et al: Impact of tumor size on outcomes after anatomic lung resection for stage 1A non-small cell lung cancer based on the current staging system. J Thorac Cardiovasc Surg 143:390-7, 2012
11. Kato T, Ishikawa K, Aragaki M, et al: Angiolymphatic invasion exerts a strong impact on surgical outcomes for stage I lung adenocarcinoma, but not non-adenocarcinoma. Lung Cancer 77:394-400, 2012
12. Petersen R P, Campa M J, Sperlazza J, et al: Tumor infiltrating Foxp3+ regulatory T-cells are associated with recurrence in pathologic stage I NSCLC patients. Cancer 107:2866-72, 2006
13. Kratz J R, He J, Van Den Eeden S K, et al: A practical molecular assay to predict survival in resected non-squamous, non-small-cell lung cancer: development and international validation studies. Lancet 379:823-32, 2012
14. Alix-Panabieres C, Schwarzenbach H, Pantel K: Circulating Tumor Cells and Circulating Tumor DNA. Annual review of medicine, 2011
15. Shedden K, Taylor J M, Enkemann S A, et al: Gene expression-based survival prediction in lung adenocarcinoma: a multi-site, blinded validation study. Nature medicine 14:822-7, 2008
16. Subramanian J, Simon R: Gene expression-based prognostic signatures in lung cancer: ready for clinical use? Journal of the National Cancer Institute 102:464-74, 2010
17. Xie Y, Minna J D: Predicting the future for people with lung cancer. Nature medicine 14:812-3, 2008
18. Bild A H, Yao G, Chang J T, et al: Oncogenic pathway signatures in human cancers as a guide to targeted therapies. Nature 439:353-7, 2006
19. Lossos I S, Czerwinski D K, Alizadeh A A, et al: Prediction of survival in diffuse large-B-cell lymphoma based on the expression of six genes. N Engl J Med 350:1828-37, 2004
20. Arpino G, Generali D, Sapino A, et al: Gene expression profiling in breast cancer: a clinical perspective. Breast 22:109-20, 2013
21. Paik S, Shak S, Tang G, et al: A multigene assay to predict recurrence of tamoxifen-treated, node-negative breast cancer. N Engl J Med 351:2817-26, 2004
22. Azim H A, Jr., Michiels S, Zagouri F, et al: Utility of prognostic genomic tests in breast cancer practice: The IMPAKT 2012 Working Group Consensus Statement. Ann Oncol 24:647-54, 2013
23. Benson A B, 3rd, Hamilton S R: Path toward prognostication and prediction: an evolving matrix. J Clin Oncol 29:4599-601, 2011
24. Choudhury A D, Eeles R, Freedland S J, et al: The role of genetic markers in the management of prostate cancer. Eur Urol 62:577-87, 2012
25. Alizadeh A A, Gentles A J, Alencar A J, et al: Prediction of survival in diffuse large B-cell lymphoma based on the expression of 2 genes reflecting tumor and microenvironment. Blood 118:1350-8, 2011
26. Guo N L, Wan Y W, Tosun K, et al: Confirmation of gene expression-based prediction of survival in non-small cell lung cancer. Clin Cancer Res 14:8213-20, 2008
27. Gevaert O, Xu J, Hoang C D, et al: Non-small cell lung cancer: identifying prognostic imaging biomarkers by leveraging public gene expression microarray data—methods and preliminary results. Radiology 264:387-96, 2012
28. Newman A M, Cooper J B: AutoSOME: a clustering method for identifying gene expression modules without prior knowledge of cluster number. BMC Bioinformatics 11:117, 2010
29. Livak K J, Schmittgen T D: Analysis of relative gene expression data using real-time quantitative PCR and the 2(−Delta Delta C(T)) Method. methods 25:402-8, 2001
30. Rodrigues R F, Roque L, Krug T, et al: Poorly differentiated and anaplastic thyroid carcinomas: chromosomal and oligo-array profile of five new cell lines. Br J Cancer 96:1237-45, 2007
31. Rock J R, Onaitis M W, Rawlins E L, et al: Basal cells as stem cells of the mouse trachea and human airway epithelium. Proc Natl Acad Sci USA 106:12771-5, 2009
32. Charafe-Jauffret E, Ginestier C, Monville F, et al: Gene expression profiling of breast cell lines identifies potential new basal markers. Oncogene 25:2273-84, 2006
33. Parsons S F, Mallinson G, Holmes C H, et al: The Lutheran blood group glycoprotein, another member of the immunoglobulin superfamily, is widely expressed in human tissues and is developmentally regulated in human liver. Proc Natl Acad Sci USA 92:5496-500, 1995
34. Wang B Y, Huang J Y, Cheng C Y, et al: Lung cancer and prognosis in taiwan: a population-based cancer registry. J Thorac Oncol 8:1128-35, 2013
35. Goodgame B, Viswanathan A, Miller C R, et al: A clinical model to estimate recurrence risk in resected stage I non-small cell lung cancer. Am J Clin Oncol 31:22-8, 2008
36. Sun Z, Aubry M C, Deschamps C, et al: Histologic grade is an independent prognostic factor for survival in non-small cell lung cancer: an analysis of 5018 hospital- and 712 population-based cases. J Thorac Cardiovasc Surg 131:1014-20, 2006
37. Ferketich A K, Niland J C, Mamet R, et al: Smoking status and survival in the national comprehensive cancer network non-small cell lung cancer cohort. Cancer 119:847-53, 2013
38. Surveillance, Epidemiology, and End Results (SEER) Program (www.seer.cancer.gov) SEER*Stat. Database: Incidence−SEER 18 Regs Research Data+Hurricane Katrina Impacted Louisiana Cases, November 2012 Sub (1973-2010 varying)−Linked To County Attributes−Total U.S., 1969-2011 Counties, National Cancer Institute, DCCPS, Surveillance Research Program, Surveillance Systems Branch, released April 2013, based on the November 2012 submission.
39. Kohlmann A, Kipps T J, Rassenti L Z, et al: An international standardization programme towards the application of gene expression profiling in routine leukaemia diagnostics: the Microarray Innovations in LEukemia study prephase. Br J Haematol 142:802-7, 2008
40. Quon G, Haider S, Deshwar A G, et al: Computational purification of individual tumor gene expression profiles leads to significant improvements in prognostic prediction. Genome Med 5:29, 2013
41. Wistuba, I I, Behrens C, Lombardi F, et al: Validation of a Proliferation-based Expression Signature as Prognostic Marker in Early Stage Lung Adenocarcinoma. Clin Cancer Res, 2013
42. Feldser D M, Kostova K K, Winslow M M, et al: Stage-specific sensitivity to p53 restoration during lung cancer progression. Nature 468:572-5, 2010
43. Abelson J A, Murphy J D, Trakul N, et al: Metabolic imaging metrics correlate with survival in early stage lung cancer treated with stereotactic ablative radiotherapy. Lung Cancer 78:219-24, 2012
44. Dobin A, Davis C A, Schlesinger F, et al: STAR: ultrafast universal RNA-seq aligner. Bioinformatics 29:15-21, 2013
45. Trapnell C, Williams B A, Pertea G, et al: Transcript assembly and quantification by RNA-Seq reveals unannotated transcripts and isoform switching during cell differentiation. Nat Biotechnol 28:511-5, 2010

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of obtaining an NSCLC marker level representation for an individual with non small cell lung carcinoma (NSCLC), the method comprising:
(a) measuring expression levels, in a biological sample from the individual, of 2 or more NSCLC markers selected from the autosome clusters set forth in Table 8;
(b) calculating an NSCLC marker level representation based on the expression levels measured in step (a); and
(c) generating a report that includes the NSCLC marker level representation of the individual.

2. The method of claim 1, wherein the NSCLC markers are selected from MAD2L1, GINS1, SLC2A1, KRT6A, FCGRT, TNIK, BCAM, KDM6A, and FAIM32.

3. The method according to claim 2, wherein calculating an NSCLC marker level representation comprises normalizing and weighting the measured expression levels of the 2 or more NSCLC markers.

4. The method according to claim 3, wherein the normalizing comprises determining the measured expression levels of the 2 or more NSCLC markers relative to the expression levels of one or more housekeeping genes.

5. The method according to any of claims 1-4, wherein the NSCLC marker level representation is an NSCLC score, and wherein calculating the NSCLC score comprises normalizing the measured expression levels of the 2 or more NSCLC markers and calculating a molecular prognostic index (MPI) value that represents a combination of the normalized expression levels.

6. The method according to claim 5, wherein calculating the NSCLC score comprises weighting the expression levels of the 2 or more NSCLC markers and calculating a molecular prognostic index (MPI) value that represents a combination of the normalized and weighted expression levels.

7. The method according to any of claims 1-6, comprising weighting the expression levels of the 2 or more NSCLC markers, wherein the expression level of MAD2L1 is multiplied by 4; the expression level of GINS1 is multiplied by 4; the expression level of SLC2A1 is multiplied by 5; the expression level of KRT6A is multiplied by 4; the expression level of FCGRT is multiplied by 5; the expression level of TNIK is multiplied by 5; the expression level of BCAM is multiplied by 5; the expression level of KDM6A is multiplied by 5; and the expression level of FAIM3 is multiplied by 6.

8. The method according to any of claims 5-7, wherein calculating the MPI value comprises adding, subtracting, and weighting the expression levels according to the formula: 4y₁+4y₂+4y₃+5y₄−5y₅−5y₆−5y₇−5y₈−6y₉, wherein each of y₁ to y₉ are the expression levels of the corresponding NSCLC marker, and wherein y₁, y₂, and y₃ are MAD2L1, GINS1, and KRT6A; y₄ is SLC2A1; y₅, y₆, y₇, and y₈ are FCGRT, TNIK, BCAM, and KDM6A; and y₉ is FAIM3.

9. The method according to any of claims 5-8, wherein calculating the NSCLC score further comprises: integrating clinical data from the individual with the MPI value of the individual to obtain a composite risk model (CRM) value.

10. The method according to any of claims 1-9, wherein the expression level for each of the 2 or more NSCLC markers is an RNA expression level.

11. The method according to claim 9, wherein the RNA expression level is measured using at least one technique selected from: microarray, polymerase chain reaction (PCR), digital barcode analysis, or sequencing.

12. The method according to claim 11, wherein the PCR is quantitative PCR.

13. The method according to any of claims 1-12, wherein the biological sample is a tumor sample.

14. The method according to claim 13, wherein the tumor sample is a formalin-fixed paraffin-embedded tumor sample.

15. The method according to any of claims 1-14, wherein the report includes a reference NSCLC marker level representation.

16. The method according to any of claims 1-15, comprising inputting the expression levels of the 2 or more NSCLC markers into a computer comprising a processor programmed to perform the calculating step.

17. The method according to claim 16, wherein said inputting results in the computer generating the report.

18. The method according to claim 17, wherein the report is displayed to an output device at a location remote to the computer.

19. The method according to any of claims 1-18, wherein the NSCLC is a non-squamous NSCLC.

20. The method according to any of claims 1-19, wherein the NSCLC is a stage I NSCLC.

21. The method according to any of claims 1-20, wherein the measuring step comprises measuring expression levels, in the biological sample, of 5 or more NSCLC markers selected from: MAD2L1, GINS1, SLC2A1, KRT6A, FCGRT, TNIK, BCAM, KDM6A, and FAIM3.

22. The method according to any of claims 1-21, wherein the measuring step comprises measuring expression levels, in the biological sample, of 7 or more NSCLC markers selected from: MAD2L1, GINS1, SLC2A1, KRT6A, FCGRT, TNIK, BCAM, KDM6A, and FAIM3.

23. The method according to any of claims 1-22, wherein the measuring step comprises measuring expression levels, in the biological sample, of the nine NSCLC markers: MAD2L1, GINS1, SLC2A1, KRT6A, FCGRT, TNIK, BCAM, KDM6A, and FAIM3.

24. A method of providing a prognosis for an individual with non small cell lung carcinoma (NSCLC), the method comprising:
(a) measuring expression levels, in a biological sample from the individual, of 2 or more NSCLC markers selected from: MAD2L1, GINS1, SLC2A1, KRT6A, FCGRT, TNIK, BCAM, KDM6A, and FAIM3;
(b) calculating an NSCLC marker level representation using the expression levels determined in step (a);
(c) comparing the NSCLC marker level representation of the individual to a reference marker level representation; and
(d) providing a prognosis for the individual based on the comparison.

25. The method according to claim 24, wherein the expression levels of FCGRT, TNIK, BCAM, KDM6A, and FAIM3 correlate positively with a positive prognosis, and wherein the expression levels of MAD2L1, GINS1, SLC2A1, and KRT6A correlate negatively with a positive prognosis.

26. The method according to any of claims 24-25, wherein the reference marker level representation is a threshold marker level representation.

27. The method according to any of claims 24-26, wherein the threshold is a predetermined threshold.

28. The method according to any of claims 24-27, wherein the reference marker level representation is derived from an experimentally determined dataset comprising survival data and NSCLC marker level representation data for individuals with NSCLC.

29. The method according to any of claims 24-28, wherein the prognosis is a category of survival likelihood.

30. The method according to claim 29, wherein the category is high risk, intermediate risk, or low risk, wherein high risk is associated with a low likelihood of survival, low risk is associated with a high likelihood of survival, and medium risk is associated with an intermediate likelihood of survival.

31. The method according to any of claims 24-28, wherein the prognosis is a statistical likelihood of survival.

32. The method according to any of claims 24-31, comprising generating a report that includes at least one of: (i) the NSCLC marker level representation, (ii) the NSCLC marker level representation of the individual and a reference NSCLC marker level representation, (iii) the prognosis, and (iv) guidance to a clinician as to a treatment recommendation for the individual based on the prognosis.

33. The method according to any of claims 24-32, comprising inputting the expression levels of the 2 or more NSCLC markers into a computer comprising a processor programmed to perform at least one of: the calculating step, the comparing step, and the providing step.

34. The method according to claim 33, wherein said inputting results in the computer generating a report that includes at least one of: (i) the NSCLC marker level representation, (ii) the NSCLC marker level representation of the individual and a reference NSCLC marker level representation, (iii) the prognosis, and (iv) guidance to a clinician as to a treatment recommendation for the individual based on the prognosis.

35. The method according to any of claims 24-34, further comprising recommending a treatment regimen based on the prognosis.

36. The method according to claim 35, wherein the recommended treatment regimen is an aggressive treatment regimen if the predicted likelihood of survival is below a predetermined threshold.

37. The method according to claim 36, wherein the recommended aggressive treatment regimen comprises chemotherapy.

38. The method according to any of claims 34-37, comprising administering or prescribing the recommended treatment.

39. A kit for obtaining an NSCLC marker level representation for an individual with non small cell lung carcinoma (NSCLC), the kit comprising:
two or more detection elements for measuring the expression levels, in a biological sample from the individual, of 2 or more NSCLC markers selected from: MAD2L1, GINS1, SLC2A1, KRT6A, FCGRT, TNIK, BCAM, KDM6A, and FAIM3

40. The kit according to claim 39, wherein the detection element for each marker is two or more primers for the specific amplification of the corresponding marker.

41. The kit according to any of claims 39-40, wherein the amplification is quantitative amplification.

42. The kit according to any of claims 39-41, comprising an NSCLC phenotype determination element.

43. The kit according to any of claims 39-42, comprising instructions for providing a prognosis for the individual based on results from said measuring.

44. A device for obtaining an NSCLC marker level representation for an individual with non small cell lung carcinoma (NSCLC), the device comprising:
(i) an analyzing unit comprising detection agents for detecting 2 or more NSCLC markers selected from: MAD2L1, GINS1, SLC2A1, KRT6A, FCGRT, TNIK, BCAM, KDM6A, and FAIM3, wherein the analyzing unit is configured for measuring expression levels of the 2 or more NSCLC markers in a biological sample of the individual; and
(ii) an evaluation unit comprising a processor programmed to:
(a) calculate an NSCLC marker level representation using the expression levels measured by the analyzing unit; and
(b) generate a report that includes the NSCLC marker level representation of the individual.

45. The device according to claim 44, wherein calculating an NSCLC marker level representation comprises normalizing the expression levels of the 2 or more NSCLC markers.

46. The device according to claim 44 or 45, wherein the NSCLC marker level representation is an NSCLC score, and wherein calculating the NSCLC score comprises normalizing and weighting the measure expression levels of the 2 or more NSCLC markers and calculating a molecular prognostic index (MPI) value that represents a combination of the normalized and weighted expression levels.

47. The device according to any of claims 44-46, wherein the processor of the evaluation unit is programmed to compare the NSCLC marker level representation of the individual to a reference marker level representation, and to provide a prognosis for the individual based on the comparison.

48. The device according to any of claims 44-47, wherein the report further includes at least one of: (i) a reference NSCLC marker level representation, (ii) the prognosis, and (iii) guidance to a clinician as to a treatment recommendation for the individual based on the prognosis.

49. The device according to any of claims 44-48, comprising a display comprising a user interface, the display in electronic communication with the processor of the evaluation unit.

50. A computer system, the system comprising:
(i) a processor; and
(ii) memory operably coupled to the processor, wherein the memory programs the processor to:
(a) receive assay data that includes expression level measurements, from a biological sample of an individual, of 2 or more NSCLC markers selected from: MAD2L1, GINS1, SLC2A1, KRT6A, FCGRT, TNIK, BCAM, KDM6A, and FAIM3;
(b) calculate an NSCLC marker level representation of the individual using the measured expression levels; and
(c) generate a report that includes the NSCLC marker level representation of the individual.

51. The system according to claim 50, wherein the memory programs the processor to compare the NSCLC marker level representation of the individual to a reference marker level representation, and to provide a prognosis for the individual based on the comparison.

52. The system according to claim 50 or 51, wherein the report further includes at least one of: (i) a reference NSCLC marker level representation, (ii) the prognosis, and (iii) guidance to a clinician as to a treatment recommendation for the individual based on the prognosis.

53. The system according to any of claims 50-52, comprising a display comprising a user interface, the display in electronic communication with the processor.


SEQUENCE LISTING (42 sequences)

SEQ ID NO: 1 - DNA, 2470 bp, Homo sapiens
ctctggctcccagccccgcagcggccgagctgcagcccgggctcagtctccgccgccgccgtgaacatggagcccccggacgcaccggcccaggcgcgcggggccccgcggctgctgttgctcgcagtcctgctggcggcgcacccagatgcccaggcggaggtgcgcttgtctgtacccccgctggtggaggtgatgcgaggaaagtctgtcattctggactgcacccctacgggaacccacgaccattatatgctggaatggttccttaccgaccgctcgggagctcgcccccgcctagcctcggctgagatgcagggctctgagctccaggtcacaatgcacgacacccggggccgcagtcccccataccagctggactcccaggggcgcctggtgctggctgaggcccaggtgggcgacgagcgagactacgtgtgcgtggtgagggcaggggcggcaggcactgctgaggccactgcgcggctcaacgtgtttgcaaagccagaggccactgaggtctcccccaacaaagggacactgtctgtgatggaggactctgcccaggagatcgccacctgcaacagccggaacgggaacccggcccccaagatcacgtggtatcgcaacgggcagcgcctggaggtgcccgtagagatgaacccagagggctacatgaccagccgcacggtccgggaggcctcgggcctgctctccctcaccagcaccctctacctgcggctccgcaaggatgaccgagacgccagcttccactgcgccgcccactacagcctgcccgagggccgccacggccgcctggacagccccaccttccacctcaccctgcactatcccacggagcacgtgcagttctgggtgggcagcccgtccaccccagcaggctgggtacgcgagggtgacactgtccagctgctctgccggggggacggcagccccagcccggagtatacgcttttccgccttcaggatgagcaggaggaagtgctgaatgtgaatctcgaggggaacttgaccctggagggagtgacccggggccagagcgggacctatggctgcagagtggaggattacgacgcggcagatgacgtgcagctctccaagacgctggagctgcgcgtggcctatctggaccccctggagctcagcgaggggaaggtgctttccttacctctaaacagcagtgcagtcgtgaactgctccgtgcacggcctgcccacccctgccctacgctggaccaaggactccactcccctgggcgatggccccatgctgtcgctcagttctatcaccttcgattccaatggcacctacgtatgtgaggcctccctgcccacagtcccggtcctcagccgcacccagaacttcacgctgctggtccaaggctcgccagagctaaagacagcggaaatagagcccaaggcagatggcagctggagggaaggagacgaagtcacactcatctgctctgcccgcggccatccagaccccaaactcagctggagccaattggggggcagccccgcagagccaatccccggacggcagggttgggtgagcagctctctgaccctgaaagtgaccagcgccctgagccgcgatggcatctcctgtgaagcctccaacccccacgggaacaagcgccatgtcttccacttcggcaccgtgagcccccagacctcccaggctggagtggccgtcatggccgtggccgtcagcgtgggcctcctgctcctcgtcgttgctgtcttctactgcgtgagacgcaaagggggcccctgctgccgccagcggcgggagaagggggctccgccgccaggggagccagggctgagccactcggggtcggagcaaccagagcagaccggccttctcatgggaggtgcctccggaggagccaggggtggcagcgggggcttcggagacgagtgctgagccaagaacctcctagaggctgtccctggacctggagctgcaggcatcagagaaccagccctgctcacgccatgcccgcccccgccttccctcttccctcttccctctccctgcccagccctcccttccttcctctgccggcaaggcagggacccacagtggctgcctgcctccgggagggaaggagagggagggtgggtgggtgggagggggccttcctccagggaatgtgactctcccaggccccagaatagctcctggacccaagcccaaggcccagcctgggacaaggctccgagggtcggctggccggagctatttttacctcccgcctcccctgctggtccccccacctgacgtcttgctgcagagtctgacactggattcccccccctcaccccgcccctggtcccactcctgcccccgccctacctccgccccaccccatcatctgtggacactggagtctggaataaatgctgtttgtcacatcaacaccaaaaaaaaaaaaaaaaaa

SEQ ID NO: 2 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 3 - DNA, 3448 bp, Homo sapiens
ctctggctcccagccccgcagcggccgagctgcagcccgggctcagtctccgccgccgccgtgaacatggagcccccggacgcaccggcccaggcgcgcggggccccgcggctgctgttgctcgcagtcctgctggcggcgcacccagatgcccaggcggaggtgcgcttgtctgtacccccgctggtggaggtgatgcgaggaaagtctgtcattctggactgcacccctacgggaacccacgaccattatatgctggaatggttccttaccgaccgctcgggagctcgcccccgcctagcctcggctgagatgcagggctctgagctccaggtcacaatgcacgacacccggggccgcagtcccccataccagctggactcccaggggcgcctggtgctggctgaggcccaggtgggcgacgagcgagactacgtgtgcgtggtgagggcaggggcggcaggcactgctgaggccactgcgcggctcaacgtgtttgcaaagccagaggccactgaggtctcccccaacaaagggacactgtctgtgatggaggactctgcccaggagatcgccacctgcaacagccggaacgggaacccggcccccaagatcacgtggtatcgcaacgggcagcgcctggaggtgcccgtagagatgaacccagagggctacatgaccagccgcacggtccgggaggcctcgggcctgctctccctcaccagcaccctctacctgcggctccgcaaggatgaccgagacgccagcttccactgcgccgcccactacagcctgcccgagggccgccacggccgcctggacagccccaccttccacctcaccctgcactatcccacggagcacgtgcagttctgggtgggcagcccgtccaccccagcaggctgggtacgcgagggtgacactgtccagctgctctgccggggggacggcagccccagcccggagtatacgcttttccgccttcaggatgagcaggaggaagtgctgaatgtgaatctcgaggggaacttgaccctggagggagtgacccggggccagagcgggacctatggctgcagagtggaggattacgacgcggcagatgacgtgcagctctccaagacgctggagctgcgcgtggcctatctggaccccctggagctcagcgaggggaaggtgctttccttacctctaaacagcagtgcagtcgtgaactgctccgtgcacggcctgcccacccctgccctacgctggaccaaggactccactcccctgggcgatggccccatgctgtcgctcagttctatcaccttcgattccaatggcacctacgtatgtgaggcctccctgcccacagtcccggtcctcagccgcacccagaacttcacgctgctggtccaaggctcgccagagctaaagacagcggaaatagagcccaaggcagatggcagctggagggaaggagacgaagtcacactcatctgctctgcccgcggccatccagaccccaaactcagctggagccaattggggggcagccccgcagagccaatccccggacggcagggttgggtgagcagctctctgaccctgaaagtgaccagcgccctgagccgcgatggcatctcctgtgaagcctccaacccccacgggaacaagcgccatgtcttccacttcggcaccgtgagcccccagacctcccaggctggagtggccgtcatggccgtggccgtcagcgtgggcctcctgctcctcgtcgttgctgtcttctactgcgtgagacgcaaagggggcccctgctgccgccagcggcgggagaagggggctccgtgagtggcctgctatctgcaatgaccaccatagcttaaccccatccccaccctcaaccccaagctcaacccataacctcaaccacatcttatcctccaccccacatcccaccacatccacctccatccccaacccatcctcatccccaactacagccccaaacccagccccagactaatccacagccatccccaactcatcctcatccccaactgcagccccaaacccaacccagggccatccccaaacccatccccaagccaaactcaacaccatccccttccctaatcacctctcctgcccctagctcagcctcatccccaactccatgcctgtctccaatcccaaccctgcccccaatctcccctcaactccaatccataacccccttcaaaccatcctcaactaagctcctctccagccctggccacatccccatcctcccccaacctccagccccaacacccatcatccccctgagctcacccttaactccaatttatcctccaagcctatctctcaccatccagccctcacctagccatttcccaaccctagtcctccaccgtcccgaagccaacttcacccatgtccccatccccaatcttgtccccattcctgactccaaatccaacccctaagcctccccaagctcctcctcatctaaccttgtctccagccccaaccacatggccgtcctcacctccaatccgtaaccatcccagaccgatcccaaagccaactccagctacattcccacctccaaccccaatagcatctgcatccccatgcccaaccctaaccccagtgccattaccagcccctaccccatccctatgccatccccacccagcacacccccatcctcacctccatccccagccgcatcctagtcccacccacccccatcctcagttcctccccctgccgtgtgcacctccaacacgacgcctccgcccgctgcctcctccccccaggccgccaggggagccagggctgagccactcggggtcggagcaaccagagcagaccggccttctcatgggaggtgcctccggaggagccaggggtggcagcgggggcttcggagacgagtgctgagccaagaacctcctagaggctgtccctggacctggagctgcaggcatcagagaaccagccctgctcacgccatgcccgcccccgccttccctcttccctcttccctctccctgcccagccctcccttccttcctctgccggcaaggcagggacccacagtggctgcctgcctccgggagggaaggagagggagggtgggtgggtgggagggggccttcctccagggaatgtgactctcccaggccccagaatagctcctggacccaagcccaaggcccagcctgggacaaggctccgagggtcggctggccggagctatttttacctcccgcctcccctgctggtccccccacctgacgtcttgctgcagagtctgacactggattcccccccctcaccccgcccctggtcccactcctgcccccgccctacctccgccccaccccatcatctgtggacactggagtctggaataaatgctgtttgtcacatcaacaccaaaaaaaaaaaaaaaaaa

SEQ ID NO: 4 - protein, 588 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAP

SEQ ID NO: 5 - DNA, 3109 bp, Homo sapiens
agcctgagaatagttagcaaacaagggaggttgtcatttcctcatcgtcaagctttgttcctcgtgggggctagaaatctctttccagttccagattgtgaagggttcctgagtaagcagcgtgtctccatccccctctctaggggctcttggatggaccttgcactctagaagggacaatggacttctggctttggccactttacttcctgccagtatcgggggccctgaggatcctcccagaagtaaaggtagagggggagctgggcggatcagttaccatcaagtgcccacttcctgaaatgcatgtgaggatatatctgtgccgggagatggctggatctggaacatgtggtaccgtggtatccaccaccaacttcatcaaggcagaatacaagggccgagttactctgaagcaatacccacgcaagaatctgttcctagtggaggtaacacagctgacagaaagtgacagcggagtctatgcctgcggagcgggcatgaacacagaccggggaaagacccagaaagtcaccctgaatgtccacagtgaatacgagccatcatgggaagagcagccaatgcctgagactccaaaatggtttcatctgccctatttgttccagatgcctgcatatgccagttcttccaaattcgtaaccagagttaccacaccagctcaaaggggcaaggtccctccagttcaccactcctcccccaccacccaaatcacccaccgccctcgagtgtccagagcatcttcagtagcaggtgacaagccccgaaccttcctgccatccactacagcctcaaaaatctcagctctggaggggctgctcaagccccagacgcccagctacaaccaccacaccaggctgcacaggcagagagcactggactatggctcacagtctgggagggaaggccaaggatttcacatcctgatcccgaccatcctgggccttttcctgctggcacttctggggctggtggtgaaaagggccgttgaaaggaggaaagccctctccaggcgggcccgccgactggccgtgaggatgcgcgccctggagagctcccagaggccccgcgggtcgccgcgaccgcgctcccaaaacaacatctacagcgcctgcccgcggcgcgctcgtggagcggacgctgcaggcacaggggaggcccccgttcccggccccggagcgccgttgccccccgccccgctgcaggtgtctgaatctccctggctccatgccccatctctgaagaccagctgtgaatacgtgagcctctaccaccagcctgccgccatgatggaggacagtgattcagatgactacatcaatgttcctgcctgacaactccccagctatcccccaaccccaggctcggactgtggtgccaaggagtctcatctatctgctgatgtccaatacctgcttcatgtgttctcagagccctcatcacttcccatgccccatctcgactcccatccccatctatctgtgccctgagcatggctctgcccccaggtcgtcttgcacaccttggcagccccctgtagttgacaggtaagctgtaggcatgtagagcaattgtcccaatgccacttgcttcctttccaagccgtcgaacagactgtgggatttgcagagtgtttcttccatgtctttgaccacagggttgttgctgcccaggctctagatcacatggcatcaggctggggcagaggcatagctattgtctcgggcatccttcccagggttgggtcttacacaaatagaaggctcttgctctgagttatgtgacatgcctcagccccatggactaagcaggggtctggtataaaaacactcctggaaacgcctttgccctgatccaaatgttagcacttgctagtgaacgtctacttatctcaagttctatgctaaaggcaatttatcttgatgtgatgataaaccaaacttattagcaagatatgcatatatatccataaattctctttactctgtctccatcacttgatgcacataagtgccctgacctcagcatctcccctctaaaaaaaaaaaaaaaaaagtatctctttatctttcttccatagcctgacactgatatttgtgcacttaccttaactttggtctattttattcatccaaaaccattacatttcttggttttcacaaatgttccccatttcttagccagttccagacaatgtatagcaagcaggggaaggaaagcagtcaggagttcctgggtggccacggctctgcaatagcacttatgtcatggaagtgatatcccacctcctacatatactctttgcctaggtttttggaacaaggttatagtcagacactgtatctttagattgatgtcgaccacaaagttcagccagagcttgaggctagatgcacagccttgctattgggaagaaggccttttctagctgtacaacacagtctcactgggcattcatccagaaatagagaagaaagtctgccagacttgagttatgttgtcttttattagcagggaatgtcatcacagattggatagtacatccaggtgcaatgtcaccatcagcaaggtcagcttgacactcaagtggaagattagggaagaatgactaggataaaaaaaaaaggagggcaccaagggaaagggatgatggggtgagctggcgagtgtgggtgggaaatgaaatgtttattgaggatctgctttgtgctgggcactttaatccacattttatcgtttacttttcaaacagatgcaccttacccccaccccaatgctctgtccctgcagatatcagaagacagtgtgattttcatgctctgaagttcagttttacatccaagcatccctctctgttttttaacaatccaaagacaggccaaaaaaagcaccacagtttattaagtacttactaagcacccatccactgccccacactgtggcaaggattgtgaggggtaaagaagcatggggcacaatattctgctgccttcatgtaacttacagtctcacaaataaatagaacttcagttgaaatactgacattaattaaatagagttgtaataaaaaaaaaaaaaaaaaaaaaaaaaa

SEQ ID NO: 6 - protein, 390 aa, Homo sapiens
MDFWLWPLYFLPVSGALRILPEVKVEGELGGSVTIKCPLPEMHVRIYLCREMAGSGTCGTVVSTTNFIKAEYKGRVTLKQYPRKNLFLVEVTQLTESDSGVYACGAGMNTDRGKTQKVTLNVHSEYEPSWEEQPMPETPKWFHLPYLFQMPAYASSSKFVTRVTTPAQRGKVPPVHHSSPTTQITHRPRVSRASSVAGDKPRTFLPSTTASKISALEGLLKPQTPSYNHHTRLHRQRALDYGSQSGREGQGFHILIPTILGLFLLALLGLVVKRAVERRKALSRRARRLAVRMRALESSQRPRGSPRPRSQNNIYSACPRRARGADAAGTGEAPVPGPGAPLPPAPLQVSESPWLHAPSLKTSCEYVSLYHQPAAMMEDSDSDDYINVPA

SEQ ID NO: 7 - DNA, 2773 bp, Homo sapiens
agcctgagaatagttagcaaacaagggaggttgtcatttcctcatcgtcaagctttgttcctcgtgggggctagaaatctctttccagttccagattgtgaagggttcctgagtaagcagcgtgtctccatccccctctctaggggctcttggatggaccttgcactctagaagggacaatggacttctggctttggccactttacttcctgccagaatacgagccatcatgggaagagcagccaatgcctgagactccaaaatggtttcatctgccctatttgttccagatgcctgcatatgccagttcttccaaattcgtaaccagagttaccacaccagctcaaaggggcaaggtccctccagttcaccactcctcccccaccacccaaatcacccaccgccctcgagtgtccagagcatcttcagtagcaggtgacaagccccgaaccttcctgccatccactacagcctcaaaaatctcagctctggaggggctgctcaagccccagacgcccagctacaaccaccacaccaggctgcacaggcagagagcactggactatggctcacagtctgggagggaaggccaaggatttcacatcctgatcccgaccatcctgggccttttcctgctggcacttctggggctggtggtgaaaagggccgttgaaaggaggaaagccctctccaggcgggcccgccgactggccgtgaggatgcgcgccctggagagctcccagaggccccgcgggtcgccgcgaccgcgctcccaaaacaacatctacagcgcctgcccgcggcgcgctcgtggagcggacgctgcaggcacaggggaggcccccgttcccggccccggagcgccgttgccccccgccccgctgcaggtgtctgaatctccctggctccatgccccatctctgaagaccagctgtgaatacgtgagcctctaccaccagcctgccgccatgatggaggacagtgattcagatgactacatcaatgttcctgcctgacaactccccagctatcccccaaccccaggctcggactgtggtgccaaggagtctcatctatctgctgatgtccaatacctgcttcatgtgttctcagagccctcatcacttcccatgccccatctcgactcccatccccatctatctgtgccctgagcatggctctgcccccaggtcgtcttgcacaccttggcagccccctgtagttgacaggtaagctgtaggcatgtagagcaattgtcccaatgccacttgcttcctttccaagccgtcgaacagactgtgggatttgcagagtgtttcttccatgtctttgaccacagggttgttgctgcccaggctctagatcacatggcatcaggctggggcagaggcatagctattgtctcgggcatccttcccagggttgggtcttacacaaatagaaggctcttgctctgagttatgtgacatgcctcagccccatggactaagcaggggtctggtataaaaacactcctggaaacgcctttgccctgatccaaatgttagcacttgctagtgaacgtctacttatctcaagttctatgctaaaggcaatttatcttgatgtgatgataaaccaaacttattagcaagatatgcatatatatccataaattctctttactctgtctccatcacttgatgcacataagtgccctgacctcagcatctcccctctaaaaaaaaaaaaaaaaaagtatctctttatctttcttccatagcctgacactgatatttgtgcacttaccttaactttggtctattttattcatccaaaaccattacatttcttggttttcacaaatgttccccatttcttagccagttccagacaatgtatagcaagcaggggaaggaaagcagtcaggagttcctgggtggccacggctctgcaatagcacttatgtcatggaagtgatatcccacctcctacatatactctttgcctaggtttttggaacaaggttatagtcagacactgtatctttagattgatgtcgaccacaaagttcagccagagcttgaggctagatgcacagccttgctattgggaagaaggccttttctagctgtacaacacagtctcactgggcattcatccagaaatagagaagaaagtctgccagacttgagttatgttgtcttttattagcagggaatgtcatcacagattggatagtacatccaggtgcaatgtcaccatcagcaaggtcagcttgacactcaagtggaagattagggaagaatgactaggataaaaaaaaaaggagggcaccaagggaaagggatgatggggtgagctggcgagtgtgggtgggaaatgaaatgtttattgaggatctgctttgtgctgggcactttaatccacattttatcgtttacttttcaaacagatgcaccttacccccaccccaatgctctgtccctgcagatatcagaagacagtgtgattttcatgctctgaagttcagttttacatccaagcatccctctctgttttttaacaatccaaagacaggccaaaaaaagcaccacagtttattaagtacttactaagcacccatccactgccccacactgtggcaaggattgtgaggggtaaagaagcatggggcacaatattctgctgccttcatgtaacttacagtctcacaaataaatagaacttcagttgaaatactgacattaattaaatagagttgtaataaaaaaaaaaaaaaaaaaaaaaaaaa

SEQ ID NO: 8 - protein, 278 aa, Homo sapiens
MDFWLWPLYFLPEYEPSWEEQPMPETPKWFHLPYLFQMPAYASSSKFVTRVTTPAQRGKVPPVHHSSPTTQITHRPRVSRASSVAGDKPRTFLPSTTASKISALEGLLKPQTPSYNHHTRLHRQRALDYGSQSGREGQGFHILIPTILGLFLLALLGLVVKRAVERRKALSRRARRLAVRMRALESSQRPRGSPRPRSQNNIYSACPRRARGADAAGTGEAPVPGPGAPLPPAPLQVSESPWLHAPSLKTSCEYVSLYHQPAAMMEDSDSDDYINVPA

SEQ ID NO: 9 - DNA, 2835 bp, Homo sapiens
gggctcttggatggaccttgcactctagaagggacaatggacttctggctttggccactttacttcctgccagtatcgggggccctgaggatcctcccagaagtaaaggtagagggggagctgggcggatcagttaccatcaagtgcccacttcctgaaatgcatgtgaggatatatctgtgccgggagatggctggatctggaacatgtggtaccgtggtatccaccaccaacttcatcaaggcagaatacaagggccgagttactctgaagcaatacccacgcaagaatctgttcctagtggaggtaacacagctgacagaaagtgacagcggagtctatgcctgcggagcgggcatgaacacagaccggggaaagacccagaaagtcaccctgaatgtccacagtgaatacgagccatcatgggaagagcagccaatgcctgagactccaaaatggtttcatctgccctatttgttccagatgcctgcatatgccagttcttccaaattcgtaaccagagttaccacaccagctcaaaggggcaaggtccctccagttcaccactcctcccccaccacccaaatcacccaccgccctcgagtgtccagagcatcttcagtagcaggtgacaagccccgaaccttcctgccatccactacagcctcaaaaatctcagctctggaggggctgctcaagccccagacgcccagctacaaccaccacaccaggctgcacaggcagagccctctccaggcgggcccgccgactggccgtgaggatgcgcgccctggagagctcccagaggccccgcgggtcgccgcgaccgcgctcccaaaacaacatctacagcgcctgcccgcggcgcgctcgtggagcggacgctgcaggcacaggggaggcccccgttcccggccccggagcgccgttgccccccgccccgctgcaggtgtctgaatctccctggctccatgccccatctctgaagaccagctgtgaatacgtgagcctctaccaccagcctgccgccatgatggaggacagtgattcagatgactacatcaatgttcctgcctgacaactccccagctatcccccaaccccaggctcggactgtggtgccaaggagtctcatctatctgctgatgtccaatacctgcttcatgtgttctcagagccctcatcacttcccatgccccatctcgactcccatccccatctatctgtgccctgagcatggctctgcccccaggtcgtcttgcacaccttggcagccccctgtagttgacaggtaagctgtaggcatgtagagcaattgtcccaatgccacttgcttcctttccaagccgtcgaacagactgtgggatttgcagagtgtttcttccatgtctttgaccacagggttgttgctgcccaggctctagatcacatggcatcaggctggggcagaggcatagctattgtctcgggcatccttcccagggttgggtcttacacaaatagaaggctcttgctctgagttatgtgacatgcctcagccccatggactaagcaggggtctggtataaaaacactcctggaaacgcctttgccctgatccaaatgttagcacttgctagtgaacgtctacttatctcaagttctatgctaaaggcaatttatcttgatgtgatgataaaccaaacttattagcaagatatgcatatatatccataaattctctttactctgtctccatcacttgatgcacataagtgccctgacctcagcatctcccctctaaaaaaaaaaaaaaaaaagtatctctttatctttcttccatagcctgacactgatatttgtgcacttaccttaactttggtctattttattcatccaaaaccattacatttcttggttttcacaaatgttccccatttcttagccagttccagacaatgtatagcaagcaggggaaggaaagcagtcaggagttcctgggtggccacggctctgcaatagcacttatgtcatggaagtgatatcccacctcctacatatactctttgcctaggtttttggaacaaggttatagtcagacactgtatctttagattgatgtcgaccacaaagttcagccagagcttgaggctagatgcacagccttgctattgggaagaaggccttttctagctgtacaacacagtctcactgggcattcatccagaaatagagaagaaagtctgccagacttgagttatgttgtcttttattagcagggaatgtcatcacagattggatagtacatccaggtgcaatgtcaccatcagcaaggtcagcttgacactcaagtggaagattagggaagaatgactaggataaaaaaaaaaggagggcaccaagggaaagggatgatggggtgagctggcgagtgtgggtgggaaatgaaatgtttattgaggatctgctttgtgctgggcactttaatccacattttatcgtttacttttcaaacagatgcaccttacccccaccccaatgctctgtccctgcagatatcagaagacagtgtgattttcatgctctgaagttcagttttacatccaagcatccctctctgttttttaacaatccaaagacaggccaaaaaaagcaccacagtttattaagtacttactaagcacccatccactgccccacactgtggcaaggattgtgaggggtaaagaagcatggggcacaatattctgctgccttcatgtaacttacagtctcacaaataaatagaacttcagttgaaatactgacattaattaaatagagttgtaataaaaaaaaaaaaaaaaaaaaaaaaaa

SEQ ID NO: 10 - protein, 306 aa, Homo sapiens
MDFWLWPLYFLPVSGALRILPEVKVEGELGGSVTIKCPLPEMHVRIYLCREMAGSGTCGTVVSTTNFIKAEYKGRVTLKQYPRKNLFLVEVTQLTESDSGVYACGAGMNTDRGKTQKVTLNVHSEYEPSWEEQPMPETPKWFHLPYLFQMPAYASSSKFVTRVTTPAQRGKVPPVHHSSPTTQITHRPRVSRASSVAGDKPRTFLPSTTASKISALEGLLKPQTPSYNHHTRLHRQSPLQAGPPTGREDARPGELPEAPRVAATALPKQHLQRLPAARSWSGRCRHRGGPRSRPRSAVAPRPAAGV

SEQ ID NO: 11 - DNA, 1893 bp, Homo sapiens
gaaggatattaaagagcacctgcaggaattttttaaggggatgccgggggaagggcttgaaggaggaactaaggaggcttccagaagattttgattaacagcgagcgctagggaaggccacggggatacagaggggtaggaggaggatccagagacctccaagttgtggactccggaggcatcgagttcctttcccagttttgtcaagaattccctgagcggcagacaccctcaaagtaaaaaacgggaaccacagagaaggaaaaagaagaaccacaagcgttttgagaaacaaacaggctgggcctagggcctggggtgtgccctgcaactggggggtggggctggatgttcttggagtcagggagcagcagcctcccacaggatgtgagagaggaactggggtctccagtcacgggagccaggagccggccagggccgcaggcaggaagggagcgaggctgaagggaacgtcgtcctctcagcatgggggtcccgcggcctcagccctgggcgctggggctcctgctctttctccttcctgggagcctgggcgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaaggagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctggaatctccagccaagtcctccgtgctcgtggtgggaatcgtcatcggtgtcttgctactcacggcagcggctgtaggaggagctctgttgtggagaaggatgaggagtgggctgccagccccttggatctcccttcgtggagacgacaccggggtcctcctgcccaccccaggggaggcccaggatgctgatttgaaggatgtaaatgtgattccagccaccgcctgaccatccgccattccgactgctaaaagcgaatgtagtcaggcccctttcatgctgtgagacctcctggaacactggcatctctgagcctccagaaggggttctgggcctagttgtcctccctctggagccccgtcctgtggtctgcctcagtttcccctcctaatacatatggctgttttccacctcgataatataacacgagtttgggcccgaatcagtgtgttctcatcatttttcaggcaggggaggtaagggaataagtcgggggactgaatggcggctgggcctcggatctctcctacaggtaac

SEQ ID NO: 12 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 13 - DNA, 1629 bp, Homo sapiens
agcctatcaagtgtctgtaattaattaaccaactgagatccagttcaggggtgaaagttcttcaggtacgaggagggcattgttgtcagtctggaccgagcccgcagagcccctcctcggcgtcctggtcccggccgtgcccgcggtgtcccgggaggaaggggcgggccgggggtcgggaggagtcacgtgccccctcccgccccaggtcgtcctctcagcatgggggtcccgcggcctcagccctgggcgctggggctcctgctctttctccttcctgggagcctgggcgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaaggagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctggaatctccagccaagtcctccgtgctcgtggtgggaatcgtcatcggtgtcttgctactcacggcagcggctgtaggaggagctctgttgtggagaaggatgaggagtgggctgccagccccttggatctcccttcgtggagacgacaccggggtcctcctgcccaccccaggggaggcccaggatgctgatttgaaggatgtaaatgtgattccagccaccgcctgaccatccgccattccgactgctaaaagcgaatgtagtcaggcccctttcatgctgtgagacctcctggaacactggcatctctgagcctccagaaggggttctgggcctagttgtcctccctctggagccccgtcctgtggtctgcctcagtttcccctcctaatacatatggctgttttccacctcgataatataacacgagtttgggcccgaatcagtgtgttctcatcatttttcaggcaggggaggtaagggaataagtcgggggactgaatggcggctgggcctcggatctctcctacaggtaac

SEQ ID NO: 14 - protein, 365 aa, Homo sapiens; identical to SEQ ID NO: 12

SEQ ID NO: 15 - DNA, 2812 bp, Homo sapiens
agctgggctttgtggagcgctgcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacggtgtcccccgccgagaagtggagcgtgcacatcgccatgcaccctcaggtcaacatctacagcgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagaatgccagctgctactttgacatcgagtggcgtgaccggcgcatcacactgagggcgtccaatggcaagtttgtgacctccaagaagaatgggcagctggccgcctcggtggagacagcaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaagactccacaggcaaatactggacggtgggcagtgactccgcggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctgagccttatttctctggaagcggctaagggacggttgggggctgggagccctgggcgtgtagtgtaactggaatcttttgcctctcccagccacctcctcccagccccccaggagagctgggcacatgtcccaagcctgtcagtggccctccctggtgcactgtccccgaaacccctgcttgggaagggaagctgtcgggtgggctaggactgacccttgtggtgtttttttgggtggtggctggaaacagcccctctcccacgtggcagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggcccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcagccccagaaaaaaaaaaaaaaaa

SEQ ID NO: 16 - protein, 493 aa, Homo sapiens
MTANGTAEAVQIQFGLINCGNKYLTAEAFGFKVNASASSLKKKQIWTLEQPPDEAGSAAVCLRSHLGRYLAADKDGNVTCEREVPGPDCRFLIVAHDDGRWSLQSEAHRRYFGGTEDRLSCFAQTVSPAEKWSVHIAMHPQVNIYSVTRKRYAHLSARPADEIAVDRDVPWGVDSLITLAFQDQRYSVQTADHRFLRHDGRLVARPEPATGYTLEFRSGKVAFRDCEGRYLAPSGPSGTLKAGKATKVGKDELFALEQSCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 17 - DNA, 3289 bp, Homo sapiens
cgcggagcggaggccgaggcgagagcctggcgctgtaggactagaacgaaaggagtgaggcgccgagagcccagataccattttggcgtgagagctggtggttggcaaggccgcgggagtgggaagcgtccgccatgttctgcgaaaaagccatggaactgatccgcgagctgcatcgcgcgcccgaagggcaactgcctgccttcaacgaggatggactcagacaagttctggaggagatgaaagctttgtatgaacaaaaccagtctgatgtgaatgaagcaaagtcaggtggacgaagtgatttgataccaactatcaaatttcgacactgttctctgttaagaaatcgacgctgcactgtagcatacctgtatgaccgcttgcttcggatcagagcactcagatgggaatatggtagcgtcttgccaaatgcattacgatttcacatggctgctgaagaaatggagtggtttaataattataaaagatctcttgctacttatatgaggtcactgggaggagatgaaggtttggacattacacaggatatgaaaccaccaaaaagcctatatattgaagtccggtgtctaaaagactatggagaatttgaagttgatgatggcacttcagtcctattaaaaaaaaatagccagcactttttacctcgatggaaatgtgagcagctgatcagacaaggagtcctggagcacatcctgtcatgaccatgcgccgaggcacttccaggcttcactcaactcatggactcctctgtactcactctctccaccactcccttcacctccctctttgattttagaagctatagacattgtttaagataactaagaatacttggctaagaagtataatttgctaactattaaggactttctttttttaatgttgtacactattcttcctactcttttttggttttggttttgttttgtagagactgtctcactatgttgcccaagctggtctcaaactcctggcctcaagcagtcctcccaccttagcttctcaaagtgttgagatcacaggcgtgagccactgcacccggcccctactcctttttctaataagctgtatctgtaatcacagcattcctacagttgttacagtgtgttttttaaatgaaagtaaacatggttacatttgaatctcttaaataagcagtcacttggctggacaggaagaaggtagatcctgtgtgtcttgttttctggtcatgtgtattgtacaagctagagagctgaatttctgagatacacattttcaaatcacatgcaagtgaagatgatggtctgtagaaattttcagtatatataatgtttaatgacatactaatttatcatctggctatttgggaaggaaggacacacatggattttgcacatttccaccatggtggctggtgtggcttgtggctatggggtgatcaccagtatcaccactttggaaggggacagtgaaattggggctagagaaggaactttgtacagttttccctgagattcagattgactgaaaagtcacatgaagagttgattgtcttttaatggtatgttttaaacagctgacattttaaattttgatgaaatccagtttattcgtttgttcttttatgctttgggtgttgcatccgagaaatcttttcccatcccaagatcacaattttttttcctttttacttctagaagtgttataattttaagctttatactttggtctatgacccgtttttttttttgttttgttttgttttttcgtttgtttctttgttttgagatggagtcttgttctgtcacccaggctggggtgcagtggcgtgatcttggctcactgcaatctctatcccctgggttcaagtgattctcttgtctcagcctcccaagtagctgggattacaggcacaggccgccacgcctggctaatttttgtatttttagtagagacagagttttaccatgttggccaggctggtttcaaactcctgacctcaagtgacccaccttggcctcccaaagttttgggattacaagtgtgggccaccgcggccagcctatgatccattttgaatgaattttttatatggtgcaaggtgtcaatccaccttcactttttcttgggaatatagatatccagctgtttcactaccattttttgaaaggactgccctttgctctatcacctttgcatttttgttaaaaagtagttgtcaatgtatatgtgggtttatttcaggactctgttttgttccattgacctgtttttctctcctgaatgccaataccatatttgtatgtagtgtatgtaattttctaataattcttgaaacagatagtattaatgtgtcatatttttgctgttgtttgtattttttgtagagatggggtttcaccgtgttggccaggctgtgttgaactcctgagctaaagcaatacacttgcctcgtcctccccatgtgctgggattacaggcgtgagccttggtgctggcccagtgtaccacatttctttttgagatttgttttggctatgttaagtcctttgcttttgatgtgaaatttgggaacaggcagggtgtggtggcttatgcctgtaatcctagaactttgggaggcctagatgggtggatcacttgagctcaggagttccagaccagcccgggcctatggcaaaactccgtctctacaaaaaatagaaaaaattagccaggtgtggtggtgcatgcctgtagtcacagttacacggcaggctgaggtgggaggatcacttgaaccccagaggtcaagactgcagtgagctgagatcacaccactgtactccagcctgggtgacaaagtgagactctatctcaaaaagaaattaggatcaatttgtcaatttctacaacaacaacaacaaaaacccctgttgggcaccttgattgagattgcattgaatttatataaaactgttgggagaattgacatcttaataatattgagtcttctggcctataaacaaggtctgtcttcctaggtattaatgttttgtcttctatttctcttaataatcttttgtagttttcagtgtacaggtctaccatgtcagcatttcatagttttgatgctaaatggtattttaaaatttcaaattctaaccacttgttgctagtaaatagaaatacaattgatgttgaacttgtatccttcagccttgctaaactgtgagttctcatggtgtttttgtaaattacatcaacagtcatgtgttctatgaataaagagttttactccttc

SEQ ID NO: 18 - protein, 196 aa, Homo sapiens
MFCEKAMELIRELHRAPEGQLPAFNEDGLRQVLEEMKALYEQNQSDVNEAKSGGRSDLIPTIKFRHCSLLRNRRCTVAYLYDRLLRIRALRWEYGSVLPNALRFHMAAEEMEWFNNYKRSLATYMRSLGGDEGLDITQDMKPPKSLYIEVRCLKDYGEFEVDDGTSVLLKKNSQHFLPRWKCEQLIRQGVLEHILS

SEQ ID NO: 19 - DNA, 5778 bp, Homo sapiens
gtgacacaattacaacaactttgtgctggtgccggggaagtttgtgtctccaacgaatcccctcagtgctccccagccccgcgcgctccggccgttcccgccgtccccgcctgtggctgccccctgcccaaccccgcgatgtgaccctacagccgaaagccgccgctgccgacccgggggctccgcagcccctgccgccgccgccgccgccttcaccgccgccgcgttgggatttttcgtcgccgccgcccgcggcggaggaggaggcggcgataaagttggtgtgctggtcccgcgcgcagattgggggcgtcactgcgggccccggtccgagggggggtgtcggcgttggagttgtgaattcgctgcgtttccatgaaatcctgcggagtgtcgctcgctaccgccgccgctgccgccgccgctttcggtgatgaggaaaagaaaatggcggcgggaaaagcgagcggcgagagcgaggaggcgtcccccagcctgacagccgaggagagggaggcgctcggcggactggacagccgcctctttgggttcgtgagatttcatgaagatggcgccaggacgaaggccctactgggcaaggctgttcgctgctatgaatctctaatcttaaaagctgaaggaaaagtggagtctgatttcttttgtcaattaggtcacttcaacctcttattggaagattatccaaaagcattatctgcataccagaggtactacagtttacagtctgactactggaagaatgctgcctttttatatggtcttggtttggtctacttccattataatgcatttcagtgggcaattaaagcatttcaggaggtgctttatgttgatcccagcttttgtcgagccaaggaaattcatttacgacttgggcttatgttcaaagtgaacacagactatgagtctagtttaaagcattttcagttagctttggttgactgtaatccctgcactttgtccaatgctgaaattcaatttcacattgcccacttatatgaaacccagaggaaatatcattctgcaaaagaagcttatgaacaacttttgcagacagagaatctttctgcacaagtaaaagcaactgtcttacaacagttaggttggatgcatcacactgtagatctcctgggagataaagccaccaaggaaagctatgctattcagtatctccaaaagtccttggaagcagatcctaattctggccagtcctggtatttcctcggaaggtgctattcaagtattgggaaagttcaggatgcctttatatcttacaggcagtctattgataaatcagaagcaagtgcagatacatggtgttcaataggtgtgctatatcagcagcaaaatcagcccatggatgctttacaggcctatatttgtgctgtacaattggaccatggccatgctgcagcctggatggacctaggcactctctatgaatcctgcaaccagcctcaggatgccattaaatgctacttaaatgcaactagaagcaaaagttgtagtaatacctctgcacttgcagcacgaattaagtatttacaggctcagttgtgtaaccttccacaaggtagtctacagaataaaactaaattacttcctagtattgaggaggcgtggagcctaccaattcccgcagagcttacctccaggcagggtgccatgaacacagcacagcagaatacttctgacaattggagtggtggacatgctgtgtcacatcctccagtacagcaacaagctcattcatggtgtttgacaccacagaaattacagcatttggaacagctccgcgcaaatagaaataatttaaatccagcacagaaactgatgctggaacagctggaaagtcagtttgtcttaatgcaacaacaccaaatgagaccaacaggagttgcacaggtacgatctactggaattcctaatgggccaacagctgactcatcactgcctacaaactcagtctctggccagcagccacagcttgctctgaccagagtgcctagcgtctctcagcctggagtccgtcctgcctgccctgggcagcctttggccaatggacccttttctgcaggccatgttccctgtagcacatcaagaacgctgggaagtacagacactattttgataggcaataatcatataacaggaagtggaagtaatggaaacgtgccttacctgcagcgaaacgcactcactctacctcataaccgcacaaacctgaccagcagcgcagaggagccgtggaaaaaccaactatctaactccactcaggggcttcacaaaggtcagagttcacattcggcaggtcctaatggtgaacgacctctctcttccactgggccttcccagcatctccaggcagctggctctggtattcagaatcagaacggacatcccaccctgcctagcaattcagtaacacagggggctgctctcaatcacctctcctctcacactgctacctcaggtggacaacaaggcattaccttaaccaaagagagcaagccttcaggaaacatattgacggtgcctgaaacaagcaggcacactggagagacacctaacagcactgccagtgtcgagggacttcctaatcatgtccatcagatgacggcagatgctgtttgcagtcctagccatggagattctaagtcaccaggtttactaagttcagacaatcctcagctctctgccttgttgatgggaaaagccaataacaatgtgggtactggaacctgtgacaaagtcaataacatccacccagctgttcatacaaagactgataactctgttgcctcttcaccatcttcagccatttcaacagcaacaccttctccaaaatccactgagcagacaaccacaaacagtgttaccagccttaacagccctcacagtgggctacacacaattaatggagaagggatggaagaatctcagagccccatgaaaacagatctgcttctggttaaccacaaacctagtccacagatcataccatcaatgtctgtgtccatataccccagctcagcagaagttctgaaggcatgcaggaatctaggtaaaaatggcttatctaacagtagcattttgttggataaatgtccacctccaagaccaccatcttcaccataccctcccttgccaaaggacaagttgaatccacctacacctagtatttacttggaaaataaacgtgatgctttctttcctccattacatcaattttgtacaaatccgaacaaccctgttacagtaatacgtggccttgctggagctcttaagttagacctgggacttttctctactaaaactttggtggaagctaacaatgaacatatggtagaagtgaggacacagttgttgcagccagcagatgaaaactgggatcccactggaacaaagaaaatctggcattgtgaaagtaatagatctcatactacaattgctaaatatgcacagtaccaggcctcctcattccaggaatcattgagagaagaaaatgaaaaaagaagtcatcataaagaccactcagatagtgaatctacatcgtcagataattctgggaggaggaggaaaggaccctttaaaaccataaagtttgggaccaatattgacctatctgatgacaaaaagtggaagttgcagctacatgagctgactaaacttcctgcttttgtgcgtgtcgtatcagcaggaaatcttctaagccatgttggtcataccatattgggcatgaacacagttcaactatacatgaaagttccagggagcagaacaccaggtcatcaggaaaataacaacttctgttcagttaacataaatattggcccaggtgactgtgaatggtttgttgttcctgaaggttactggggtgttctgaatgacttctgtgaaaaaaataatttgaatttcctaatgggttcttggtggcccaatcttgaagatctttatgaagcaaatgttccagtgtataggtttattcagcgacctggagatttggtctggataaatgcaggcactgttcattgggttcaggctattggctggtgcaacaacattgcttggaatgttggtccacttacagcctgccagtataaattggcagtggaacggtacgaatggaacaaattgcaaagtgtgaagtcaatagtacccatggttcatctttcctggaatatggcacgaaatatcaaggtctcagatccaaagctttttgaaatgattaagtattgtcttctaagaactctgaagcaatgtcagacattgagggaagctctcattgctgcaggaaaagagattatatggcatgggcggacaaaagaagaaccagctcattactgtagcatttgtgaagtggaggtttttgatctgctttttgtcactaatgagagtaattcacgaaagacctacatagtacattgccaagattgtgcacgaaaaacaagcggaaacttggaaaactttgtggtgctagaacagtacaaaatggaggacctgatgcaagtctatgaccaatttacattagctcctccattaccatccgcctcatcttgatattgttccatggacattaaatgagaccttttctgctattcaggaaataacccagttctgcaccactggtttttgtagctatctcgtaaggctgctggctgaaaactgtgtctatgcaaccttccaagtgcggagtgtcaaccaactggacgggagagagtactgctcctactccaggactctcacaaagctgatgagctgtacttcagaaaaaaataataatttccatgttttgtatatatctgacaaaactggcaacatcttacagactactgacttgaagacaacctcttttatatttctctatttctgggctgatgaatttgttttcatctgtcttttcccccttcagaattttccttggaaaaaaaatactagcctagctggtcatttctttgtaaggtagttagcaattttaagtctttctttggtcaacttttttttaatgtgaaaagttaggtaagacacttttttactgcttttatgtttttctgtcttgttttgagaccatgatggttacacttttggttcctaaataaaatttaaaaaattaacagccaagtcacaaaggtaatggattgcacatagactaaggaataaacttcagatttgtgatttttgtttctaatcttgatgtaaatttacactatttataaatacatatttattgcttgaaaatatttgtgaatggaatgctgttattttttccagatttacctgccattgaaattttaaggagttctgtaatttcaaacactactcctattacattttctatgtgtaaataaaactgcttagcattgtacagaaacttttattaaaattgtttaatgtttaaagagttttctattgtttgagttttaaaaaagactttatgtacagtgcccagtttttgttcatttttgaaatctgattatatatattttatatatacttatgtatgtatatataatatatatagaaatctggatatatatgtataaatctttagaacttaaatttttctcgttttaagtttcacatctatggtagatttttgaggtgtctactgtaaagtattgcttacaaaaagtatgattatttttaaagaaatatatatggtatgtatcctcaagacctaaaatgtcagactggtttattgttaagttgcaattactgcaatgacagaccaataaacaattgctgccaaaaaaaaaa

SEQ ID NO: 20 - protein, 1401 aa, Homo sapiens
MKSCGVSLATAAAAAAAFGDEEKKMAAGKASGESEEASPSLTAEEREALGGLDSRLFGFVRFHEDGARTKALLGKAVRCYESLILKAEGKVESDFFCQLGHFNLLLEDYPKALSAYQRYYSLQSDYWKNAAFLYGLGLVYFHYNAFQWAIKAFQEVLYVDPSFCRAKEIHLRLGLMFKVNTDYESSLKHFQLALVDCNPCTLSNAEIQFHIAHLYETQRKYHSAKEAYEQLLQTENLSAQVKATVLQQLGWMHHTVDLLGDKATKESYAIQYLQKSLEADPNSGQSWYFLGRCYSSIGKVQDAFISYRQSIDKSEASADTWCSIGVLYQQQNQPMDALQAYICAVQLDHGHAAAWMDLGTLYESCNQPQDAIKCYLNATRSKSCSNTSALAARIKYLQAQLCNLPQGSLQNKTKLLPSIEEAWSLPIPAELTSRQGAMNTAQQNTSDNWSGGHAVSHPPVQQQAHSWCLTPQKLQHLEQLRANRNNLNPAQKLMLEQLESQFVLMQQHQMRPTGVAQVRSTGIPNGPTADSSLPTNSVSGQQPQLALTRVPSVSQPGVRPACPGQPLANGPFSAGHVPCSTSRTLGSTDTILIGNNHITGSGSNGNVPYLQRNALTLPHNRTNLTSSAEEPWKNQLSNSTQGLHKGQSSHSAGPNGERPLSSTGPSQHLQAAGSGIQNQNGHPTLPSNSVTQGAALNHLSSHTATSGGQQGITLTKESKPSGNILTVPETSRHTGETPNSTASVEGLPNHVHQMTADAVCSPSHGDSKSPGLLSSDNPQLSALLMGKANNNVGTGTCDKVNNIHPAVHTKTDNSVASSPSSAISTATPSPKSTEQTTTNSVTSLNSPHSGLHTINGEGMEESQSPMKTDLLLVNHKPSPQIIPSMSVSIYPSSAEVLKACRNLGKNGLSNSSILLDKCPPPRPPSSPYPPLPKDKLNPPTPSIYLENKRDAFFPPLHQFCTNPNNPVTVIRGLAGALKLDLGLFSTKTLVEANNEHMVEVRTQLLQPADENWDPTGTKKIWHCESNRSHTTIAKYAQYQASSFQESLREENEKRSHHKDHSDSESTSSDNSGRRRKGPFKTIKFGTNIDLSDDKKWKLQLHELTKLPAFVRVVSAGNLLSHVGHTILGMNTVQLYMKVPGSRTPGHQENNNFCSVNINIGPGDCEWFVVPEGYWGVLNDFCEKNNLNFLMGSWWPNLEDLYEANVPVYRFIQRPGDLVWINAGTVHWVQAIGWCNNIAWNVGPLTACQYKLAVERYEWNKLQSVKSIVPMVHLSWNMARNIKVSDPKLFEMIKYCLLRTLKQCQTLREALIAAGKEIIWHGRTKEEPAHYCSICEVEVFDLLFVTNESNSRKTYIVHCQDCARKTSGNLENFVVLEQYKMEDLMQVYDQFTLAPPLPSASS

SEQ ID NO: 21 - DNA, 2450 bp, Homo sapiens
atatttcatacctttctagaaactgggtgtgatctcactgttggtaaagcccagcccttcccaacctgcaagctcaccttccaggactgggcccagcccatgctctccatatataagctgctgccccgagcctgattcctagtcctgcttctcttccctctctcctccagcctctcacactctcctcagctctctcatctcctggaaccatggccagcacatccaccaccatcaggagccacagcagcagccgccggggtttcagtgccaactcagccaggctccctggggtcagccgctctggcttcagcagcgtctccgtgtcccgctccaggggcagtggtggcctgggtggtgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcaccgtcaaccagagtctcctgactcccctcaacctgcaaatcgatcccaccatccagcgggtgcgggctgaggagcgtgaacagatcaagaccctcaacaacaagtttgcctccttcatcgacaaggtgcggttcctggagcagcagaacaaggttctggaaacaaagtggaccctgctgcaggagcagggcaccaagactgtgaggcagaacctggagccgttgttcgagcagtacatcaacaacctcaggaggcagctggacagcattgtcggggaacggggccgcctggactcagagctcagaggcatgcaggacctggtggaggacttcaagaacaaatatgaggatgaaatcaacaagcgcacagcagcagagaatgaatttgtgactctgaagaaggatgtggatgctgcctacatgaacaaggttgaactgcaagccaaggcagacactctcacagacgagatcaacttcctgagagccttgtatgatgcagagctgtcccagatgcagacccacatctcagacacatctgtggtgctgtccatggacaacaaccgcaacctggacctggacagcatcatcgctgaggtcaaggcccaatatgaggagattgctcagagaagccgggctgaggctgagtcctggtaccagaccaagtacgaggagctgcaggtcacagcaggcagacatggggacgacctgcgcaacaccaagcaggagattgctgagatcaaccgcatgatccagaggctgagatctgagatcgaccacgtcaagaagcagtgcgccaacctgcaggccgccattgctgatgctgagcagcgtggggagatggccctcaaggatgccaagaacaagctggaagggctggaggatgccctgcagaaggccaagcaggacctggcccggctgctgaaggagtaccaggagctgatgaatgtcaagctggccctggacgtggagatcgccacctaccgcaagctgctggagggtgaggagtgcaggctgaatggcgaaggcgttggacaagtcaacatctctgtggtgcagtccaccgtctccagtggctatggcggtgccagtggtgtcggcagtggcttaggcctgggtggaggaagcagctactcctatggcagtggtcttggcgttggaggtggcttcagttccagcagtggcagagccattgggggtggcctcagctctgttggaggcggcagttccaccatcaagtacaccaccacctcctcctccagcaggaagagctataagcactaaagtgcgtctgctagctctcggtcccacagtcctcaggcccctctctggctgcagagccctctcctcaggttgcctttcctctcctggcctccagtctcccctgctgtcccaggtagagctgggtatggatgcttagtgccctcacttcttctctctctctctataccatctgagcacccattgctcaccatcagatcaacctctgattttacatcatgatgtaatcaccactggagcttcactgttactaaattattaatttcttgcctccagtgttctatctctgaggctgagcattataagaaaatgacctctgctccttttcattgcagaaaattgccaggggcttatttcagaacaacttccacttactttccactggctctcaaactctctaacttataagtgttgtgaacccccacccaggcagtatccatgaaagcacaagtgactagtcctatgatgtacaaagcctgtatctctgtgatgatttctgtgctcttcgctgtttgcaattgctaaataaagcagatttataatacaata

SEQ ID NO: 22 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 23 - DNA, 3687 bp, Homo sapiens
tccaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggccactcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctggcaagaggcaagaggtagcaacagcgagcgtgccggtcgctagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccccactctaggggtcaggctccattaggatttgccccttcccatctcttcctacccaaccactcaaattaatctttctttacctgagaccagttgggagcactggagtgcagggaggagaggggaagggccagtctgggctgccgggttctagtctcctttgcactgagggccacactattaccatgagaagagggcctgtgggagcctgcaaactcactgctcaagaagacatggagactcctgccctgttgtgtatagatgcaagatatttatatatatttttggttgtcaatattaaatacagacactaagttatagtatatctggacaagccaacttgtaaatacaccacctcactcctgttacttacctaaacagatataaatggctggtttttagaaacatggttttgaaatgcttgtggattgagggtaggaggtttggatgggagtgagacagaagtaagtggggttgcaaccactgcaacggcttagacttcgactcaggatccagtcccttacacgtacctctcatcagtgtcctcttgctcaaaaatctgtttgatccctgttacccagagaatatatacattctttatcttgacattcaaggcatttctatcacatatttgatagttggtgttcaaaaaaacactagttttgtgccagccgtgatgctcaggcttgaaatgcattattttgaatgtgaagtaaatactgtacctttattggacaggctcaaagaggttatgtgcctgaagtcgcacagtgaataagctaaaacacctgcttttaacaatggtaccatacaaccactactccattaactccacccacctcctgcacccctccccacacacacaaaatgaaccacgttctttgtatgggcccaatgagctgtcaagctgccctgtgttcatttcatttggaattgccccctctggttcctctgtatactactgcttcatctctaaagacagctcatcctcctccttcacccctgaatttccagagcacttcatctgctccttcatcacaagtccagttttctgccactagtctgaatttcatgagaagatgccgatttggttcctgtgggtcctcagcactattcagtacagtgcttgatgcacagcaggcactcagaaaatactggaggaaataaaacaccaaagatatttgtcaaaaaaaaaaaaaaaaa

SEQ ID NO: 24 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 25 - DNA, 5816 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatacatcaggcgacagttagaggaggagcagagacagttagagatcttgcagcagcagctactgcatgaacaagctctacttctggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggaggtagaagaacggtcaaggctcaaccggcaaagttcccctgccatgcctcacaaggttgccaacaggatatctgaccccaacctgcccccaaggtcggagtccttcagcattagtggagttcagcctgctcgaacaccccccatgctcagaccagtcgatccccagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctagctacaaaaaagctatagatgaggatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 26 - protein, 1360 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 27 - DNA, 5792 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatacatcaggcgacagttagaggaggagcagagacagttagagatcttgcagcagcagctactgcatgaacaagctctacttctggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggaggtagaagaacggtcaaggctcaaccggcaaagttcccctgccatgcctcacaaggttgccaacaggatatctgaccccaacctgcccccaaggtcggagtccttcagcattagtggagttcagcctgctcgaacaccccccatgctcagaccagtcgatccccagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctgatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 28 - protein, 1352 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPADLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 29 - DNA, 5729 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggaggtagaagaacggtcaaggctcaaccggcaaagttcccctgccatgcctcacaaggttgccaacaggatatctgaccccaacctgcccccaaggtcggagtccttcagcattagtggagttcagcctgctcgaacaccccccatgctcagaccagtcgatccccagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctagctacaaaaaagctatagatgaggatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 30 - protein, 1331 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 31 - DNA, 5705 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggaggtagaagaacggtcaaggctcaaccggcaaagttcccctgccatgcctcacaaggttgccaacaggatatctgaccccaacctgcccccaaggtcggagtccttcagcattagtggagttcagcctgctcgaacaccccccatgctcagaccagtcgatccccagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctgatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 32 - protein, 1323 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPADLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 33 - DNA, 5651 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatacatcaggcgacagttagaggaggagcagagacagttagagatcttgcagcagcagctactgcatgaacaagctctacttctggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctagctacaaaaaagctatagatgaggatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 34 - protein, 1305 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 35 - DNA, 5627 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatacatcaggcgacagttagaggaggagcagagacagttagagatcttgcagcagcagctactgcatgaacaagctctacttctggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctgatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 36 - protein, 1297 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPADLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 37 - DNA, 5564 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctagctacaaaaaagctatagatgaggatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 38 - protein, 1276 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYKRKQLEEQRQAERLQRQLKQERDYLVSLQHQRQEQRPVEKKPLYHYKEGMSPSEKPAWAKEIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 39 - DNA, 5540 bp, Homo sapiens
attataacagccaatgcagccgccatccacgcacaagcaaagaagcatgtcccatttaaatacacccacgcagccccagcgcccttagccgatcggggcgctcagcccacacgcaccgctgctcggggcttggagatccgcgcaggctgggctcccggacccggcggaccgacgcgcggaggatcgggatccggcgctgtggggctggggtgggcgggggaggctgggcccggggcctctggcgcgacacccgcatgaggacgcgagtgaaatagaccaaggtggaatttccaagggaaaagcttcggggtggttttggtccatttctccagcgaagaagtagacatggcgagcgactccccggctcgaagcctggatgaaatagatctctcggctctgagggaccccgcagggatctttgaattggtggaacttgttggaaatggaacatacgggcaagtttataagggtcgtcatgtcaaaacgggccagcttgcagccatcaaggttatggatgtcacaggggatgaagaggaagaaatcaaacaagaaattaacatgttgaagaaatattctcatcaccggaatattgctacatactatggtgcttttatcaaaaagaacccaccaggcatggatgaccaactttggttggtgatggagttttgtggtgctggctctgtcaccgacctgatcaagaacacaaaaggtaacacgttgaaagaggagtggattgcatacatctgcagggaaatcttacgggggctgagtcacctgcaccagcataaagtgattcatcgagatattaaagggcaaaatgtcttgctgactgaaaatgcagaagttaaactagtggactttggagtcagtgctcagcttgatcgaacagtgggcaggaggaatactttcattggaactccctactggatggcaccagaagttattgcctgtgatgaaaacccagatgccacatatgatttcaagagtgacttgtggtctttgggtatcaccgccattgaaatggcagaaggtgctccccctctctgtgacatgcaccccatgagagctctcttcctcatcccccggaacccagcgcctcggctgaagtctaagaagtggtcaaaaaaattccagtcatttattgagagctgcttggtaaagaatcacagccagcgaccagcaacagaacaattgatgaagcatccatttatacgagaccaacctaatgagcgacaggtccgcattcaactcaaggaccatattgatagaacaaagaagaagcgaggagaaaaagatgagacagagtatgagtacagtggaagtgaggaagaagaggaggagaatgactcaggagagcccagctccatcctgaatctgccaggggagtcgacgctgcggagggactttctgaggctgcagctggccaacaaggagcgttctgaggccctacggaggcagcagctggagcagcagcagcgggagaatgaggagcacaagcggcagctgctggccgagcgtcagaagcgcatcgaggagcagaaagagcagaggcggcggctggaggagcaacaaaggcgagagaaggagctgcggaagcagcaggagagggagcagcgccggcactatgaggagcagatgcgccgggaggaggagaggaggcgtgcggagcatgaacaggaatataagcgcaaacaattggaagaacagagacaagcagaaagactgcagaggcagctaaagcaagaaagagactacttagtttcccttcagcatcagcggcaggagcagaggcctgtggagaagaagccactgtaccattacaaagaaggaatgagtcctagtgagaagccagcatgggccaaggagatcccacatctggtagctgtaaaatcccagggacctgccttgaccgcctcccagtcagtgcacgagcagcccacaaagggcctctctgggtttcaggaggctctgaacgtgacctcccaccgcgtggagatgccacgccagaactcagatcccacctcggaaaatcctcctctccccactcgcattgaaaagtttgaccgaagctcttggttacgacaggaagaagacattccaccaaaggtgcctcaaagaacaacttctatatccccagcattagccagaaagaattctcctgggaatggtagtgctctgggacccagactaggatctcaacccatcagagcaagcaaccctgatctccggagaactgagcccatcttggagagccccttgcagaggaccagcagtggcagttcctccagctccagcacccctagctcccagcccagctcccaaggaggctcccagcctggatcacaagcaggatccagtgaacgcaccagagttcgagccaacagtaagtcagaaggatcacctgtgctcccccatgagcctgccaaggtgaaaccagaagaatccagggacattacccggcccagtcgaccagctgatctgacggcattagccaaagaactaagagaactccggattgaagaaacaaaccgcccaatgaagaaggtgactgattactcctcctccagtgaggagtcagaaagtagcgaggaagaggaggaagatggagagagcgagacccatgatgggacagtggctgtcagcgacatacccagactgataccaacaggagctccaggcagcaacgagcagtacaatgtgggaatggtggggacgcatgggctggagacctctcatgcggacagtttcagcggcagtatttcaagagaaggaaccttgatgattagagagacgtctggagagaagaagcgatctggccacagtgacagcaatggctttgctggccacatcaacctccctgacctggtgcagcagagccattctccagctggaaccccgactgagggactggggcgcgtctcaacccattcccaggagatggactctgggactgaatatggcatggggagcagcaccaaagcctccttcaccccctttgtggaccccagagtataccagacgtctcccactgatgaagatgaagaggatgaggaatcatcagccgcagctctgtttactagcgaacttcttaggcaagaacaggccaaactcaatgaagcaagaaagatttcggtggtaaatgtaaacccaaccaacattcggcctcatagcgacacaccagaaatcagaaaatacaagaaacgattcaactcagaaatactttgtgcagctctgtggggtgtaaaccttctggtggggactgaaaatggcctgatgcttttggaccgaagtgggcaaggcaaagtctataatctgatcaaccggaggcgatttcagcagatggatgtgctagagggactgaatgtccttgtgacaatttcaggaaagaagaataagctacgagtttactatctttcatggttaagaaacagaatactacataatgacccagaagtagaaaagaaacaaggctggatcactgttggggacttggaaggctgtatacattataaagttgttaaatatgaaaggatcaaatttttggtgattgccttaaagaatgctgtggaaatatatgcttgggctcctaaaccgtatcataaattcatggcatttaagtcttttgcagatctccagcacaagcctctgctagttgatctcacggtagaagaaggtcaaagattaaaggttatttttggttcacacactggtttccatgtaattgatgttgattcaggaaactcttatgatatctacataccatctcatattcagggcaatatcactcctcatgctattgtcatcttgcctaaaacagatggaatggaaatgcttgtttgctatgaggatgagggggtgtatgtaaacacctatggccggataactaaggatgtggtgctccaatggggagaaatgcccacgtctgtggcctacattcattccaatcagataatgggctggggcgagaaagctattgagatccggtcagtggaaacaggacatttggatggagtatttatgcataagcgagctcaaaggttaaagtttctatgtgaaagaaatgataaggtattttttgcatccgtgcgatctggaggaagtagccaagtgtttttcatgaccctcaacagaaattccatgatgaactggtaacagaagagcacttggcacttatcttcatggcgttatttctaatttaaaagaacataactcatgtggacttatgccagtctagaggcagaatcagaaggcttggttgaacatatcgctttccctttttcctctccctccgcccctcccagtacagtccatctttcaatgttgcagcctggttgagaaggagagaaaaaggtggcaggaatttccaggagatccccaagaatgctgccttgtctgtggacaaagatggaccatgtgcccttcggaattagggatagaaacaaatattgtgtgctcttaacgattaagctgtgttatggtgggttttcaggtttttaccttttttctttacccctttactctgcaagaatggggaaagaatgcatactgcgaaaatgagtcttttaaattctgtctgcctactagttttaagtatatggtatgttgtaaaatttccaatgatgagagacagcacaataaatgtaccttatctccttaggctgaaggccataactacatagtggagtaatttaagaactctcttgccttcaccaacccaaaaggttgctttttgatagcaactggctaatgaatttttaaaaagagaagaaaaatactagttttcccctcttttgggaaatagattttaaatggctaaactactagccttaaaactactagtctaataaaatcaactaccacttttgtgaatctgacaggccacatttttatatggccctttacagaatggagtgtgttgaacaggatactaacgccattgagttgagctggcctagcgatggagggacactctaacacaactttccctcagctattatgcaacagatcagggaaaaagatgggatgacagatggggtcagacagaaagagcttctgggaaacaagcttacatagtcttttttaaaatgcacaaagcctcccagctaagaggtcacttggtttgggcttcattaggactgagactttgttgagttctttctgggacttggagagtggatgatattcaggctctgaacattcccagcgctctcccgagggtgccactttctcaagatgaaaactgtgactgaaaaaattaataataaatgtttctgagctgcctgtgttctccctgtgtgggtgagagaagggactagactcctaagcctgcctcagatacaagaggcatcattggctccaattttagagaacttgaaagcaaggctttggacaaaattttgagaccctaatcactttaccttcctccaaattacccaacatacggtaaacaacatttgtgcagaagtatgtatgtatttagttcaggttgacttgtgtccttataaactcttactcaaatgatttgaactttaaaaaaaaaa

SEQ ID NO: 40 - protein, 1268 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYKRKQLEEQRQAERLQRQLKQERDYLVSLQHQRQEQRPVEKKPLYHYKEGMSPSEKPAWAKEIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPADLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 41 - DNA, 1453 bp, Homo sapiens
cgcaaaggacctgacgacgtgctgcgtcgttacttttgaaacgcttggcggggaagtgctgttggagccgctgtggttgctgtccgcggagtggaagcgcgtgcttttgtttgtgtccctggccatggcgctgcagctctcccgggagcagggaatcaccctgcgcgggagcgccgaaatcgtggccgagttcttctcattcggcatcaacagcattttatatcagcgtggcatatatccatctgaaacctttactcgagtgcagaaatacggactcaccttgcttgtaactactgatcttgagctcataaaatacctaaataatgtggtggaacaactgaaagattggttatacaagtgttcagttcagaaactggttgtagttatctcaaatattgaaagtggtgaggtcctggaaagatggcagtttgatattgagtgtgacaagactgcaaaagatgacagtgcacccagagaaaagtctcagaaagctatccaggatgaaatccgttcagtgatcagacagatcacagctacggtgacatttctgccactgttggaagtttcttgttcatttgatctgctgatttatacagacaaagatttggttgtacctgaaaaatgggaagagtcgggaccacagtttattaccaattctgaggaagtccgccttcgttcatttactactacaatccacaaagtaaatagcatggtggcctacaaaattcctgtcaatgactgaggatgacatgaggaaaataatgtaattgtaattttgaaatgtggttttcctgaaatcaagtcatctatagttgatatgttttatttcattggttaatttttacatggagaaaaccaaaatgatacttactgaactgtgtgtaattgttccttttatttttttggtacctatttgacttaccatggagttaacatcatgaatttattgcacattgttcaaaaggaaccaggaggtttttttgtcaacattgtgatgtatattcctttgaagatagtaactgtagatggaaaaacttgtgctataaagctagatgctttcctaaatcagatgttttggtcaagtagtttgactcagtataggtagggagatatttaagtataaaatacaacaaaggaagtctaaatattcagaatctttgttaaggtcctgaaagtaactcataatctataaacaatgaaatattgctgtatagctccttttgaccttcatttcatgtatagttttccctattgaatcagtttccaattatttgactttaatttatgtaacttgaacctatgaagcaatggatatttgtactgtttaatgttctgtgatacagaactcttaaaaatgttttttcatgtgttttataaaatcaagttttaagtgaaagtgaggaaataaagttaagtttgttttaaatttgtcttaaaaaaaaaaaaaaaaaaa

SEQ ID NO: 42 - protein, 205 aa, Homo sapiens
MALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVND